(19) United States
(12) Patent Application Publication
(10) Pub. No.: US 2023/0219898 A1
(43) Pub. Date: Jul. 13, 2023

(54) COMPOUNDS AND METHODS FOR BLOCKING APOPTOSIS AND INDUCING AUTOPHAGY

(71) Applicant: President and Fellows of Harvard College, Cambridge, MA (US)

(72) Inventors: Junying Yuan, Waban, MA (US); Heng Zhao, Dedham, MA (US); Daichao Xu, Brookline, MA (US); Mingzhi Jin, Shanghai (CN); Hong Zhu, San Diego, CA (US); Gregory D. Cuny, Houston, TX (US)

(21) Appl. No.: 18/010,642

(22) PCT Filed: Jun. 16, 2021
(86) PCT No.: PCT/US21/37621

§ 371 Date: Dec. 15, 2022

Related U.S. Application Data
(60) Provisional application No. 63/039,905, filed on Jun. 16, 2020.

(51) Int. Cl. C07D 233/42; C07D 233/26; C07C 335/32; C07D 401/12; C07D 295/195; C07D 207/08; A61K 31/4178; A61K 31/4184

(52) U.S. Cl. CPC C07D 233/42; C07D 233/26; C07C 335/32; C07D 401/12; C07D 295/195; C07D 207/08; A61K 31/4178; A61K 31/4184

(57) ABSTRACT

Disclosed herein are small molecules that inhibit apoptosis and promote autophagy through the TRADD pathway, and their use for treatment of neurodegenerative diseases. Methods of preparing these small molecules and medicinal efficacy are described.

### RELATED APPLICATION

This application claims priority to U.S. Provisional Patent Application Ser. No. 63/039,905, filed Jun. 16, 2020, the contents of which are hereby incorporated by reference.

### BACKGROUND

Apoptosis and accumulation of misfolded proteins are both implicated in mediating human degenerative and inflammatory diseases.

Apoptosis is a caspase-mediated cellular suicide pathway in metazoan and can be activated to mediate acute tissue injuries and diseases such as stroke, heart attack and spinal cord injuries as well as in neurodegenerative diseases associated with aging. Apoptosis can be activated by TNFα and other cognate ligands of the death receptor family. The stimulation of TNFR1 by TNFα triggers the rapid formation of complex I associated with the intracellular death domain (DD) of TNFR1. Two intracellular DD containing proteins, adaptor protein TRADD and a kinase RIPK1, are recruited into complex I by DD-mediated homotypic interactions with the DD of TNFR1.

TRADD is a 34 kDa protein that contains N-terminal TRAF2 binding domain (N-TRADD, a.a. 1-169) and a C-terminal death domain (DD, 195-312). TRADD is involved in mediating both activation of NF-κB and cell death in cells stimulated by TNFα. TRADD is essential for the activation of RIPK1-dependent apoptosis (RDA).

Pathways involving TRADD include modulating ubiquitination of RIPK1, and TNFR1 collectively promotes the recruitment and activation of TBK1, TAK1 and IKK to mediate the activation of NF-κB pathway. TBK1 and TAK1 as well as the downstream kinases activated by TAK1 including IKK and MK2 are important for suppressing RIPK1 activation to block RIPK1-dependent apoptosis. Aging human brains show significant reduction of TAK1, suggesting that the increased vulnerability to RDA may be involved in mediating the onset of common neurodegenerative diseases associated with aging.

Autophagy, an intracellular degradative mechanism, can be activated to remove misfolded proteins. Autophagy is a catabolic process mediating the turnover of intracellular constituents in a lysosome-dependent manner. In metazoans, autophagy functions as an essential intracellular catabolic mechanism involved in cellular homeostasis by mediating the turnover of malfunctioning, aged or damaged proteins and organelles.

Accumulation of misfolded and neurotoxic proteins is a common feature of human neurodegenerative diseases such as Alzheimer's disease, Parkinson's disease, Huntington's disease and amyotrophic lateral sclerosis. Activation of autophagy leads to the formation of double membraned autophagosomes which sequester large protein oligomers and aggregates and degrades them. Promoting the removal of misfolded proteins is considered as the goal of potential therapeutic strategies for neurodegeneration. Activating autophagy and inhibiting apoptosis may also promote healthy aging.

Development of pharmacological inhibitors of caspases to block apoptosis for the treatment of human diseases has been challenging. Engaging appropriate molecular targets for developing pharmacological inhibitors to block apoptosis as direct inhibition of caspases can promote necroptosis. Due to this necroptosis, researchers in the field of cell death have been seeking inhibitors of apoptosis for the past 3-4 decades without success.

Molecular targets that can be effectively modulated to activate autophagy have not been identified. Therefore, an urgent need exists for pharmaceuticals that can both inhibit apoptosis without deleterious side effects and effectively promote autophagy to develop effective treatments for neurodegenerative disorders.

### SUMMARY

The invention relates, in part, to compounds that both inhibit apoptosis and activate autophagy, compositions comprising such compounds, and methods of using such compounds and compositions.

Provided herein are compounds of formula I, and pharmaceutically acceptable salts thereof:

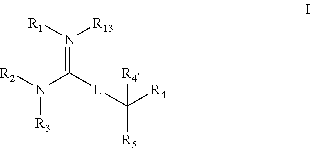

wherein
L is CH₂, NR₁ₐ, heteroaryl or S(O)n, where n is 0, 1, or 2;
R₁ₐ is independently selected from H, CN, alkyl, and aryl;
R₃ is selected from H, alkyl, and aryl;
R₄ is selected from H, alkyl, and aryl;
R_{4′} is selected from H, alkyl, and aryl;
R₅ is selected from H, alkyl, aryl, heteroaryl, —(CH₂)ₚCONR₆R₇ where p is 0, 1, or 2,
CH₂NR₆R₇, —CH(OH)NR₆R₇, —CH(OH)CH₂-cycloalkyl, —CH(OH)CH₂—NHcycloalkyl, and —CR₁₀═CHR₁₁;
   R₆ is selected from H, alkyl, C₃₋₈cycloalkyl, aryl, and -NHcycloalkyl;
   R₇ is selected from H and alkyl;
or R₆ and R₇, taken together with the nitrogen atom to which they are attached, form a heterocyclyl;
   R₁₀ is selected from H and halo;
   R₁₁ is cycloalkyl;
   R₁₃ is absent or alkyl, where the alkyl forms an iminium group; and
   (a) R₁ and R₂ are each independently selected from H, CN, alkyl, and aryl; or
   (b) R₁ and R₂, taken together with the atoms to which they are attached, form a heterocyclyl of Formula IA:

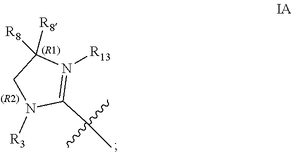

R₈ and R_{8′} are each independently selected from H, alkyl, and aryl; or
(c) R₁ and R₂, taken together with the atoms to which they are attached, and R₃ and R₄, taken together with the atoms to which they are attached, form a bicycle of Formula IB:

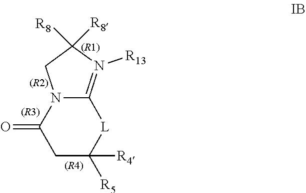

R₈ and R_{8′} are each independently selected from H, alkyl, and aryl;

further wherein when R₅ is —(CH₂)₀CONR₆R₇, then R₇ and R₄, taken together with the atoms to which they are attached, may form a heterocyclyl of Formula IC:

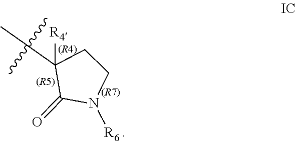

Further provided herein are compounds of formula II:

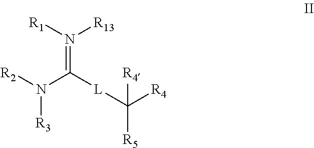

wherein

### L is NR₁ₐ or S;

R₁ₐ is independently selected from CN, alkyl, and aryl;
(a) R₁ and R₂ are each independently selected from CN, alkyl, and aryl, or
(b) R₁ and R₂, taken together with the atoms to which they are attached, form a heterocyclyl of Formula IIA:

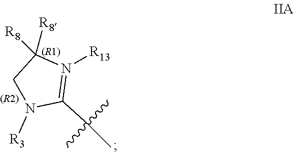

wherein R₈ and R_{8′} are each H or alkyl;

R₃ is selected from H, alkyl, and aryl;
R₄ is selected from H, alkyl, and aryl;
R_{4′} is selected from H, alkyl, and aryl;
R₅ is selected from aryl, —(CH₂)ₚCONR₆R₇ where p is 0 or 2, —CH₂NR₆R₇, —CH(OH)NR₆R₇, and —CR₁₀═CHR₁₁;
R₆ is selected from alkyl, aryl, and C₃₋₈cycloalkyl, such as C₃₋₄cycloalkyl or C₇₋₈cycloalkyl;
R₇ is selected from H and alkyl,
or R₆ and R₇, taken together with the nitrogen atom to which they are attached, form a heterocyclyl;

### R₁₀ is H;

R₁₁ is cycloalkyl;
R₁₃ is absent or alkyl, where the alkyl forms an iminium group,
or a pharmaceutically acceptable salt thereof.

In some embodiments, the compound of Formula II is not:

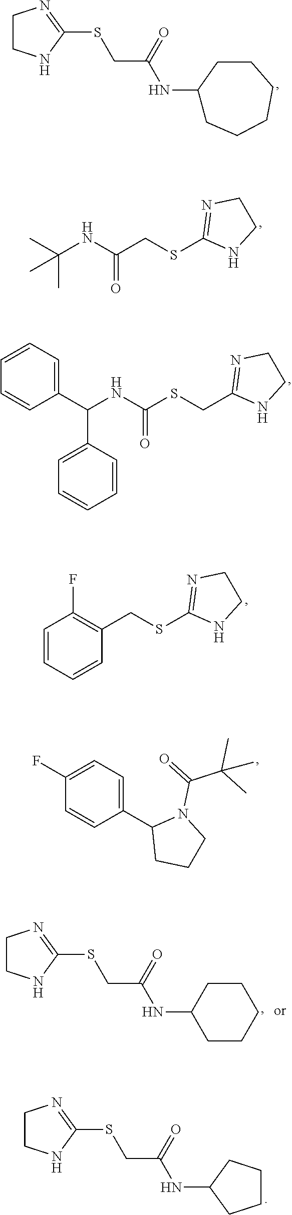

Some embodiments of the invention relate to a pharmaceutical composition comprising a compound of formula I or II, or a pharmaceutically acceptable salt, biologically active metabolite, solvate, hydrate, prodrug, enantiomer or stereoisomer thereof, and one or more pharmaceutically acceptable carriers, alone or in combination with another therapeutic agent. Such pharmaceutical compositions of the invention can be administered in accordance with a method of the invention, typically as part of a therapeutic regimen for treatment or prevention of conditions and disorders related to cancer or pancreatitis.

Certain embodiments of the invention relate to a method of treating neurodegenerative diseases, liver diseases, ischemic brain injury, inflammatory bowel diseases, amyloidosis (e.g., peripheral amyloidosis), muscular dystrophy, and metabolic diseases in a subject in need thereof, comprising administering to a subject in need thereof an effective amount (e.g., a therapeutically effective amount) of one or more compounds or pharmaceutical compositions of the invention.

### BRIEF DESCRIPTION OF THE FIGURES

FIG. 1a shows the chemical structures of ICCB-17, ICCB-19, ICCB-19i, and Apt-1.

FIG. 1b shows representative images and quantification of GFP-LC3 puncta in H4-GFP-LC3 cells treated as indicated. Mean ±s.e.m. (n=5). One-way ANOVA, post hoc Dunnett's test.

FIG. 1c contains a scatter plot depicting interactome changes of Beclin 1 from quantitative proteomics experiment. The targets are depicted as large red dots.

FIG. 1d shows immunoprecipitation-immunoblot of MEFs.

FIG. 1e shows immunoblot and quantification of LC3 II and p62 levels in MEFs of the indicated genotypes treated with indicated compounds. Mean ±s.e.m. (n=3).

FIG. if shows K63 ubiquitination of Beclin 1 from MEFs of indicated genotypes treated with Apt-1.

FIG. 1g shows K63 ubiquitination of Beclin 1 from MEFs of indicated genotypes treated with Apt-1.

FIG. 1h shows immunoblot and quantification of LC3 II and p62 levels in MEFs of the reconstituted H4 cells treated with indicated compounds. Mean ±s.e.m. (n=3).

FIG. 1i shows K63 ubiquitination of Beclin 1 from MEFs of reconstituted H4 cells. Compounds treated at 10 μM, 6 h.

FIG. 2a shows RIPK1 ubiquitination and activation in MEFs treated with indicated compounds.

FIG. 2b shows the effect of ICCB-19 treated cells. Complex I was isolated and analyzed by mass spectrometry. Red dots: significant changes in ICCB-19-treated cells. Green/black dots: no change.

FIG. 2c shows RIPK1 ubiquitination and activation in MEFs treated with indicated compounds.

FIG. 2d shows cell survival in MEFs of indicated genotypes treated with indicated compounds. Mean ±s.d. (n=3). Two-way ANOVA, post hoc Bonferroni's tests.

FIG. 2e shows immunoblot and quantification of CC3 (cleaved caspase-3) in MEFs of indicated genotypes treated with indicated compounds. Mean ±s.e.m. (n=3).

FIG. 2f shows cell survival in Jurkat of indicated genotypes treated with indicated compounds. Mean ±s.d. (n=3). Two-way ANOVA, post hoc Bonferroni's tests.

FIG. 2g shows immunoblot and quantification of LC3 II in Jurkat of indicated genotypes treated with indicated compounds. Mean ±s.e.m. (n=3).

FIG. 2h shows immunoprecipitation-immunoblot of Jurkat cells of indicated genotypes.

FIG. 2i shows K63 ubiquitination of Beclin 1 from control and reconstituted Tradd^{-/-}MEFs treated with Apt-1. Compounds treated at 10 μM, 6 h or indicated.

FIG. 3a shows immunoblots of tau levels, showing the effects of Apt-1 on the pathological tangle-like tau aggregates in the hippocampus CA1 region of PS19 mice injected with tau pffs.

FIG. 3b shows immunostaining of phospho-tau (AT8), showing the effects of Apt-1 on the pathological tangle-like tau aggregates in the hippocampus CA1 region of PS19 mice injected with tau pffs.

FIG. 3c shows immunohistochemistry for tau in pathological conformation (MC1) in hippocampus CA1 region, showing the effects of Apt-1 on the pathological tangle-like tau aggregates in the hippocampus CA1 region of PS19 mice injected with tau pffs.

FIG. 3d shows immunostaining of p-RIPK1(S166), showing the effects of Apt-1 on the pathological tangle-like tau aggregates in the hippocampus CA1 region of PS19 mice injected with tau pffs.

FIG. 3e shows immunostaining of TUNEL, showing the effects of Apt-1 on the pathological tangle-like tau aggregates in the hippocampus CA1 region of PS19 mice injected with tau pffs. Each dot represents the mean from an individual mouse. Mean ±s.e.m. (n=3). Two-tailed t-test.

FIG. 4a shows the effect of Apt-1 on TRADD-N TRAF2-C binding by NanoBiT assay.

FIG. 4b shows immunoprecipitation-immunoblot of MEFs treated with Apt-1.

FIG. 4c shows in vitro binding of Apt-1 to His-TRADD-N WT and indicated mutants as determined by thermal shift assay.

FIG. 4d shows the kinetic profile of Apt-1 binding to TRADD-N from SPR analysis.

FIG. 4e shows the binding pose of Apt-1 in complex with TRADD-N generated by induced-fit docking. Left, shape and polarity of the ligand binding pocket surface (red, negatively charged; blue, positively charged). Right, details of the interaction. Apt-1 shown as cyan sticks, protein shown as pink cartoon with key residues highlighted in sticks. Dashed lines represent hydrogen bonds.

FIG. 4f shows in vitro binding of Apt-1 to His-TRADD-N WT and indicated mutants as determined by thermal shift assay.

FIG. 4g shows immunoblot and quantification of LC3 II in reconstituted Tradd^{-/-} MEFs treated with Apt-1. Mean ±s.e.m. (n=3). Two-way ANOVA, post hoc Bonferroni's tests. Compounds treated at 10 μM, 6 h.

FIG. 5a shows a multiplex chemical screening scheme for compounds that can modulate cellular homeostasis by activating autophagy and also block apoptosis. Primary screen: Jurkat cells were treated with Velcade (50 nM) and individual compounds (10 μM) in the library for 25 h and cell viability was measured. 710 compounds which could protect against Velcade-induced apoptosis were selected. Secondary counterscreen: HCT116 cells were treated with 5-fluorouracil (5-FU) (100 μM) and individual compounds selected from the Primary screen (10 μM) for 24 h and cell viability was measured. The hits which protected against apoptosis induced by 5-FU were eliminated from further studies. Tertiary screen: H4-GFP-LC3 cells were treated with individual compounds (10 μM) for 24 h and GFP-LC3 dots were quantified. Quaternary screen: RGC-5 cells were treated with mTNFα (0.5 ng/ml), TAK1 inhibitor (5Z)-7-Oxozeanol (0.5 μM) and individual compounds (10 μM) for 8 h and cell viability was measured.

FIG. 5b shows IC50s of ICCB-19 and Apt-1 protecting Velcade-induced apoptosis (50 nM) in Jurkat cells treated with indicated compounds for 24 h and cell viability was measured.

FIG. 5c shows IC50s of ICCB-19 and Apt-1 protecting RDA in MEFs were treated with mTNFα (1 ng/mL) and 5Z-7-Oxozeaenol (0.5 μM) in the presence of indicated compounds at different concentrations for 8 h and cell survival was measured.

FIG. 5d depicts the KINOMEscan profiling of Apt-1 (10 μM) against a panel of 97 kinases. Binding interactions reported as % Ctrl, where lower numbers indicate stronger hits. Negative control=DMSO (100% Ctrl); positive control=control compound (0% Ctrl); 0<% Ctrl <0.1 Very Strong; 0.1≤% Ctrl <1 Strong; 1≤% Ctrl <10 Medium; 10 K % Ctrl ≤35 Weak; % Ctrl ≥35 No effects. No significant binding of Apt-1 to this panel of 97 kinases was detected. CellTiter-Glo was used to determine cell survival in (a), (b), and (c).

FIG. 6a shows H4 cells were treated with indicated concentrations of Apt-1. Autophagy was determined by LC3II levels using immunoblotting. SE=shorter exposure, LE=longer exposure.

FIG. 6b shows SH-SY5Y cells were treated with Apt-1 (10 μM), NH₄Cl (20 mM) as indicated for 6 h. Autophagy was measured by LC3II induction and p62 reduction by immunoblotting. The levels of LC3II in cells treated with both Apt-1 and NH₄Cl, the latter of which inhibits lysosome, were higher than that treated with either Apt-1 or NH₄Cl alone. Thus, ICCB-19/Apt-1, but not ICCB-19i, induce autophagic flux. Mean ±s.e.m. from technical triplicates (n=3), representative of 3 independent experiments. Two-tailed t-test. **P=0.0037.

FIG. 6c shows HeLa cells were treated with Apt-1 (10 μM), NH₄Cl (20 mM) as indicated for 6 h. Autophagy was measured by LC3II induction and p62 reduction by immunoblotting. The levels of LC3II in cells treated with both Apt-1 and NH₄Cl, the latter of which inhibits lysosome, were higher than that treated with either Apt-1 or NH₄Cl alone. Thus, ICCB-19/Apt-1, but not ICCB-19i, induce autophagic flux. Mean ±s.e.m. from technical triplicates (n=3), representative of 3 independent experiments. Two-tailed t-test. **P=0.0024.

FIG. 6d shows HT-29 cells were treated with Apt-1 (10 μM), NH₄Cl (20 mM) as indicated for 6 h. Autophagy was measured by LC3II induction and p62 reduction by immunoblotting. The levels of LC3II in cells treated with both Apt-1 and NH₄Cl, the latter of which inhibits lysosome, were higher than that treated with either Apt-1 or NH₄Cl alone. Thus, ICCB-19/Apt-1, but not ICCB-19i, induce autophagic flux. Mean ±s.e.m. from technical triplicates (n=3), representative of 3 independent experiments. Two-tailed t-test. **P=0.0027.

FIG. 6e shows Jurkat cells were treated with Apt-1 (10 μM), NH₄Cl (20 mM) as indicated for 6 h. Autophagy was measured by LC3II induction and p62 reduction by immunoblotting. The levels of LC3II in cells treated with both Apt-1 and NH₄Cl, the latter of which inhibits lysosome, were higher than that treated with either Apt-1 or NH₄Cl alone. Thus, ICCB-19/Apt-1, but not ICCB-19i, induce autophagic flux. Mean ±s.e.m. from technical triplicates (n=3), representative of 3 independent experiments. Two-tailed t-test. **P=0.0036.

FIG. 6f shows the effects of ICCB-19/Apt-1 on long-lived protein degradation. The rates of long-lived protein turnover in H4 cells treated with indicated compounds (10 μM, 6 h); rapamycin as positive control. Values expressed as fold changes relative to normal control cells. Mean ±s.e.m. from 4 independent experiments (n=4). One-way ANOVA, post hoc Dunnett's test. **P=0.002, 0.0023 (left to right); *P=0.0229; n.s. not significant (P=0.8669).

FIG. 6g demonstrates MEFs and Jurkat cells treated with zVAD.fmk (20 μM) for 6 h. Levels of LC3II determined by immunoblotting.

FIG. 6h shows MEFs treated with vehicle (0 h), ICCB-19 (10 μM), or Apt-1 (10 μM) for indicated times. Cell lysates analyzed by immunoblotting using indicated antibodies.

FIG. 6i shows H4-DsRed-FYVE cells that were treated with indicated compounds for 6 h and imaged; representative cells shown. Average DsRed-FYVE puncta per 1000 cells from each sample was determined using ImageJ. Mean ±s.e.m. of the puncta per cell from 5 independent experiments (n=5). One-way ANOVA, post hoc Dunnett's test. **P 0.0034; ***P=0.0004; n.s. not significant (P=0.6502).

FIG. 6j Beclin 1/Vps34 kinase complex isolated from Flag-Beclin 1 transfected HEK293T cells treated with ICCB-19, Apt-1, or ICCB-19i (10 μM) for 6 h. PI3P kinase activity was measured by in vitro lipid kinase assay using ADP-Glo Kinase Assay Kit. Wortmannin (10 μM) was used as a control to inhibit Vps34 kinase activity. Mean±s.d. from technical quadruplicates (n=4), representative of 3 independent experiments. One-way ANOVA, post hoc Dunnett's tests. ***P=0.0003; ***P<0.001 (left to right).

FIG. 7a shows HEK29T cells that were transfected with Flag-Beclin 1 for 12 h, then treated with Apt-1 (10 μM) for another 12 h. Cell lysates were immunoprecipitated using anti-Flag beads. cIAP1 and TRAF2 levels were determined by immunoblotting.

FIG. 7b shows MEFs that were treated with indicated concentrations of Apt-1 for 12 h. Cell lysates were immunoprecipitated using anti-Beclin 1 antibody. cIAP1 and TRAF2 levels were determined by immunoblotting.

FIG. 7c shows long-lived protein turnover rates in MEFs with indicated genotypes treated with indicated compounds. Expressed as fold changes relative to normal control cells. Mean ±s.d. from technical quadruplicates (n=4), representative of 3 independent experiments. Two-way ANOVA, post hoc Bonferroni's tests. ***P<0.001.

FIG. 7d shows long-lived protein turnover rates in MEFs with indicated genotypes treated with indicated compounds. Expressed as fold changes relative to normal control cells. Mean ±s.d. from technical quadruplicates (n=4), representative of 3 independent experiments. Two-way ANOVA, post hoc Bonferroni's tests. ***P<0.001.

FIG. 7e shows MEFs that were pre-treated with SM-164 (1 μM) for 1 h, then treated with Apt-1 for 6 h. LC3II levels were determined by immunoblotting. Mean ±s.e.m. are quantified from 3 independent experiments (n=3). Two-tailed t-test. ***P=0.0003.

FIG. 7f shows shRNA-mediated TRAF2 stable knockdown MEFs that were treated with Apt-1 (10 μM) for 6 h. LC3II levels were determined by immunoblotting. Mean s.e.m. are quantified from 3 independent experiments (n=3). Two-tailed t-test. ***P=0.0003.

FIG. 7g shows clap1^{-/-} and Traf2−/− MEFs reconstituted with HA-mcIAP1 and HA-mTRAF2, respectively, that were treated with Apt-1 (10 μM) for 6 h. LC3II levels were determined by immunoblotting. Mean s.e.m. are quantified from 3 independent experiments (n=3). Two-tailed t-test. ***P=0.0057.

FIG. 7h shows clap1^{-/-} and Traf2−/− MEFs reconstituted with HA-mcIAP1 and HA-mTRAF2, respectively, that were treated with Apt-1 (10 μM) for 6 h. LC3II levels were determined by immunoblotting. Mean s.e.m. are quantified from 3 independent experiments (n=3). Two-tailed t-test. ***P=0.0007 (h).

FIG. 7i shows MEFs with indicated genotypes that were treated with rapamycin (1 M) for indicated time. LC3II levels were determined by immunoblotting.

FIG. 7j shows MEFs with indicated genotypes that were treated with rapamycin (1 M) for indicated time. LC3II levels were determined by immunoblotting.

FIG. 7k shows MEFs cells with indicated genotypes that were incubated in HBSS for indicated time. LC3II levels were determined by immunoblotting. The quantification of each experiment was shown on the right.

FIG. 7l shows MEFs cells with indicated genotypes that were incubated in HBSS for indicated time. LC3II levels were determined by immunoblotting. The quantification of each experiment was shown on the right.

FIG. 7m shows MEFs that were treated with indicated compounds for 6 h, then cell lysates were tandem-immunoprecipitated with anti-Beclin 1 antibody and denatured in 3M urea. Anti-K63-linkage specific polyubiquitin antibody was used to conduct secondary immunoprecipitation. Samples were then immunoblotted with anti-Beclin 1 antibody to measure the K63-linkage specific ubiquitination of Beclin 1.

FIG. 7n shows MEFs that were pretreated with SM-164 (1 μM) for 1 h, then treated with Apt-1 (10 μM) for 6 h, then K63-linkage specific ubiquitination of Beclin 1 was analyzed as in FIG. 7m.

FIG. 7o shows reconstituted MEFs were treated with Apt-1 (10 μM) for 6 h, then K63-linkage specific ubiquitination of Beclin 1 was analyzed as in FIG. 7m.

FIG. 7p shows reconstituted MEFs were treated with Apt-1 (10 μM) for 6 h, then K63-linkage specific ubiquitination of Beclin 1 was analyzed as in FIG. 7m.

FIG. 8a depicts a schematic representation of mass spectrometry assay to determine K63 ubiquitination sites of Beclin 1 by cIAP1.

FIG. 8b shows a quantitative mass spec analysis of K63 ubiquitination of each lysine site.

FIG. 8c shows the sequence alignment of key ubiquitination sites (K) within Beclin 1 orthologs from different species.

FIG. 8d shows HEK293T cells that were transfected with indicated plasmids for 24 h. Cells were lysed in 6 M urea and lysates were subjected to pull-down with Ni²⁺ beads and analyzed by immunoblotting with anti-Beclin 1 antibody to detect ubiquitylated Beclin 1.

FIG. 8e shows validation of Beclin 1 expression in Beclin 1-silenced H4 cells.

FIG. 8f shows control and Beclin 1-silenced H4 cells that were treated with Apt-1 (10 M) for 6 h. LC3II levels were determined by immunoblotting.

FIG. 8g shows Beclin 1-silenced H4 cells reconstituted with WT and mutants Beclin 1 that were treated with Apt-1 (10 μM) for 6 h. LC3II levels were determined by immunoblotting. For FIGS. 8d-8g, mean±s.e.m. are quantified from 3 independent experiments in graphs (n=3). Two-tailed t-test. **P=0.0022 (in FIG. 8d), 0.0049 (in FIG. 8f), 0.0024 (in FIG. 8g); *P=0.0309, 0.0195 (left to right, in FIG. 8d), 0.0126 (in FIG. 8g); n.s. not significant, (P=0.6959) (in FIG. 8f).

FIG. 9a shows Jurkat cells that were stimulated by Velcade (50 nM) in the presence of Apt-1 (10 μM), Nec-1s (10 μM), or zVAD (20 μM) for 12 h and 24 h. The levels of cleaved caspase-3 were determined by immunoblotting.

FIG. 9b shows SH-SY5Y cells that were stimulated by Velcade (50 nM) in the presence of Apt-1 (10 μM), Nec-1s (10 μM), or zVAD (20 μM) for 12 h and 24 h. The levels of cleaved caspase-3 were determined by immunoblotting.

FIG. 9c shows Takl^{-/-} MEFs that were treated with 1 ng/ml mTNFα in the presence of indicated compounds for 3 h. Cell viability was determined using CellTiter-Glo assay. Mean ±s.d. from technical triplicates (n=3) representative of 3 independent experiments. One-way ANOVA, post hoc Dunnett's tests. ***P<0.001; n.s. not significant, (P=0.7989).

FIG. 9d shows Takl^{-/-} MEFs that were treated as in FIG. 9a, the cell lysates were analyzed by immunoblotting using indicated antibodies.

FIG. 9e shows MEFs that were treated with mTNFα (1 ng/ml) and 5Z-7-Oxozeaenol (0.5 μM) in the presence of indicated compounds for 1 h and 2 h and the cell lysates were analyzed by immunoblotting using indicated antibodies.

FIG. 9f shows that ICCB-19/Apt-1 inhibit RDA, including complex IIa formation. MEFs treated as in FIG. 9e were lysed with IP buffer and FADD was immunoprecipitated by anti-FADD antibody. Total lysates and IP samples were analyzed by immunoblotting to determine the recruitment of RIPK1 to FADD in complex IIa.

FIG. 9g shows that ICCB-19/Apt-1 inhibit RDA, including caspase-8 activation. MEFs were treated with mTNFα (1 ng/ml) and 5Z-7-Oxozeaenol (0.5 μM) in the presence of ICCB-19 (10 μM), Apt-1 (10 μM), Nec-1s (10 μM), or zVAD.fmk (20 μM) for 4 h and the activity of caspase-8 was determined using Caspase-Glo 8 Assay Systems. Mean ±s.d. from technical triplicates (n=3) representative of 3 independent experiments. One-way ANOVA, post hoc Dunnett's tests. ***P<0.001.

FIG. 10a shows RDA was induced in Tbk1^{-/-} MEFs by the treatment with mTNFα (10 ng/ml) together with ICCB-19 (10 μM) and Nec-1s (10 μM) at indicated times and cell death was determined by SYTOX Green.

FIG. 10b shows RDA was induced in Tbk1^{-/-} MEFs by the treatment with mTNFα (10 ng/ml) together with ICCB-19 (10 μM) and Nec-1s (10 μM) at indicated times and cell death was determined caspase-3 cleavage (CC3) immunoblotting.

FIG. 10c shows RDA was induced in Nemo^{-/-} MEFs by the treatment with mTNFα (10 ng/ml) together with ICCB-19 (10 μM) and Nec-1s (10 μM) at indicated times and cell death was determined by SYTOX Green.

FIG. 10d shows RDA was induced in Nemo^{-/-} MEFs by the treatment with mTNFα (10 ng/ml) together with ICCB-19 (10 μM) and Nec-1s (10 μM) at indicated times and cell death was determined by caspase-3 cleavage (CC3) immunoblotting.

FIG. 10e shows that RDA was induced in WT MEFs by the treatment with mTNFα (10 ng/ml) and IKK inhibitor TPCA-1 (5 μM) in the presence of ICCB-19 (10 μM) or Nec-1s (10 μM) for indicated times and cell death was determined by SYTOX Green. Mean ±s.d. from technical triplicates (n=3), representative of 3 independent experiments. Two-way ANOVA. ***P<0.001.

FIG. 10f shows recombinant active caspase-8 was incubated with vehicle, ICCB-19 (10 μM), Apt-1 (10 μM), or zVAD.fmk (20 μM) for 1 h and the activity of caspase-8 was determined using Caspase-Glo 8 Assay Systems. Mean ±s.d. from technical triplicates (n=3), representative of 3 independent experiments. One-way ANOVA, post hoc Dunnett's tests. n.s. not significant, (P=0.9931, 0.9215 (left to right)).

FIG. 10g shows WT MEFs that were treated with mTNFα (1 ng/ml) and cycloheximide (CHX, 1 μg/mL) to induce RIA in the presence or absence of ICCB-19 (10 μM) or Nec-1s (10 μM) for indicated time and cell survival was determined by CellTiter-Glo assay. Mean ±s.d. from technical octuplicates (n=8), representative of 3 independent experiments. One-way ANOVA, post hoc Dunnett's tests. n.s. not significant, (P=0.9962).

FIG. 10h shows p65/p50 DKO MEFs that were treated with mTNFα (1 ng/ml) together with ICCB-19 (10 μM) for indicated times and cell survival was determined by CellTiter-Glo assay. Mean ±s.d. from technical triplicates (n=3), representative of 3 independent experiments. Two-way ANOVA. n.s. not significant, (P=0.1895).

FIG. 10i shows MEFs that were treated as indicated and the cell survival was measured by CellTiter-Glo assay. The concentrations of reagents used: mTNFα: 1 ng/mL; (5Z)-7-oxozeaenol: 0.5 μM; zVAD: 20 μM; ICCB-19: 10 μM; Apt-1: 10 μM; Nec-1s: 10 μM. Mean s.d. from technical triplicates (n=3), representative of 3 independent experiments.

FIG. 10j shows the necroptosis of MEFs was induced by the treatment with TNFα/5z7/zVAD in the presence of indicated compounds for indicated hours and the activation of RIPK1(p-S166), RIPK3(p-T231/5232), and MLKL(p-S345) was determined by immunoblotting.

FIG. 10k shows HEK293T cells that were transfected with Flag-RIPK1 expression construct for 12 h in the presence of Nec-1s (10 μM), ICCB-19 (10 μM), or Apt-1 (10 μM). The activation of RIPK1 was determined by immunoblotting using p-S166 RIPK1 antibody.

FIG. 11a shows the mass spectrometry analysis of FIG. 2b, using ICCB-19, was confirmed by immunoprecipitation-immunoblotting using indicated antibodies, quantified on the right.

FIG. 11b shows the mass spectrometry analysis of FIG. 2b, using Apt-1 was confirmed by immunoprecipitation-immunoblotting using indicated antibodies, quantified on the right.

FIG. 11c shows MEFs that were treated with Flag-mTNFα (50 ng/ml) in the presence of Apt-1 (10 μM) for indicated time. The complex I was isolated by anti-Flag beads and denatured in 6 M urea. The complex I was further analyzed by immunoprecipitation using anti-M1 (6 M urea) or K63 (3 M urea) ubiquitin antibody under denatured condition. The levels of RIPK1 ubiquitination were analyzed by immunoblotting.

FIG. 11d shows WT and Traf2^{-/-} MEFs that were stimulated by mTNFα (1 ng/ml) and 5Z-7-Oxozeaenol (0.5 μM) in the presence of indicated compounds for 8 h. Cell survival was determined by CellTiter-Glo assay. Mean ±s.d. from technical quadruplicates (n=4), representative of 3 independent experiments. Two-way ANOVA, post hoc Bonferroni's tests. ***P<0.001.

FIG. 11e shows WT and clap1/2^{-/-} MEFs that were stimulated by mTNFα (10 ng/ml) in the presence of vehicle, ICCB-19 (10 μM) or Nec-1s (10 μM) for indicated time. Cell death was determined by CellTiter-Glo assay. Mean ±s.d. from technical triplicates (n=3), representative of 3 independent experiments. Two-way ANOVA.

FIG. 11f shows MEFs that were pretreated with SM-164 (50 nM) for 1 h, then stimulated by mTNFα (10 ng/ml) in the presence of vehicle, ICCB-19 (10 μM) or Nec-1s (10 μM) for indicated time. Cell death was determined by SYTOX Green assay. Mean±s.d. from technical triplicates (n=3), representative of 3 independent experiments. Two-way ANOVA. ***P<0.001; n.s. not significant, (P=0.1772).

FIG. 11g shows clap1/2^{-/-} MEFs that were stimulated with Flag-TNF (50 ng/ml) for indicated minutes in the presence of vehicle or ICCB-19 (10 μM) and the complex I was pulled down using anti-Flag beads. The levels of activated RIPK1 and total RIPK1 were determined by immunoblotting.

FIG. 11h shows clap1/2^{-/-} MEFs that were stimulated with Flag-TNF (50 ng/ml) for indicated minutes in the presence of vehicle or Apt-1 (10 μM) and the complex I was pulled down using anti-Flag beads. TRADD recruitment to complex I was determined by immunoblotting, quantified on the right.

FIG. 11i shows cIAP1-reconstituted cIAP1/2 DKO MEFs that were stimulated with Flag-TNF (50 ng/ml) for indicated minutes in the presence of vehicle or Apt-1 (10 μM) and the complex I was pulled down using anti-Flag beads. TRADD recruitment to complex I was determined by immunoblotting, quantified on the right.

FIG. 11j shows Fadd-deficient and Ripk1-deficient Jurkat cells were treated with Velcade (50 nM) in the presence of ICCB-19 (10 μM), Nec-1s (10 μM), NAC (100 μM), or zVAD.fmk (20 μM). The activation of caspase-8, PARP cleavage were determined by immunoblotting.

FIG. 11k shows Fadd-deficient and Ripk1-deficient Jurkat cells were treated with Velcade (50 nM) in the presence of ICCB-19 (10 μM), Nec-1s (10 μM), NAC (100 μM), or zVAD.fmk (20 μM). The activation of caspase-3 was determined by immunoblotting.

FIG. 12a shows Tradd^{+/+} and Tradd^{-/-} MEFs were treated with vehicle or ICCB-19 (10 μM) for 6 h. Autophagy levels were determined by immunoblotting using anti-LC3 antibody. Mean ±s.e.m. are quantified from 3 independent experiments (right) (n=3). Two-tailed t-test. n.s. not significant, (P=0.9172).

FIG. 12b shows Tradd^{+/+} and Tradd^{-/-} MEFs were treated with Apt-1 (10 μM), NH₄C₁ (10 mM) as indicated for 6 h. Autophagy levels were determined by immunoblotting using anti-LC3 antibody. Mean ±s.e.m. are quantified from 3 independent experiments (right) (n =3). Two-tailed t-test. n.s. not significant (P=0.4064, 0.8913 (left to right)).

FIG. 12c shows long-lived protein turnover rates in Tradd^{+/+} and Tradd^{-/-} MEFs. Expressed as fold changes relative to Tradd^{+/+} cells. Mean ±s.e.m. from 5 biological replicates (n=5). Two-tailed t-test. ***P<0.001.

FIG. 12d shows WT and Tradd-KO Jurkat cells were treated with Apt-1 (10 μM) and Spautin-1 (10 μM) followed by Velcade (50 nM) for 24 h. Cell survival was determined by CellTiter-Glo assay. Mean ±s.d. from technical quadruplicates (n=4), representative of 3 independent experiments. Two-way ANOVA, post hoc Bonferroni's tests. ***P<0.001.

FIG. 12e shows Jurkat cells that were treated with ICCB-19 (10 μM), Apt-1 (10 μM), Chloroquine (50 μM), E64d (5 μg/ml) followed by Velcade (50 nM) for 24 h. The cell survival was determined by CellTiter-Glo assay.

FIG. 12f shows Atg5-WT and Atg5-KO Jurkat cells that were pretreated with Apt-1 (10 μM) or zVAD (20 μM) for 1 h, then stimulated by Velcade (50 μM) for 24 h. Cell survival was determined by CellTiter-Glo assay. Validation of Atg5 knockout was determined by immunoblotting, quantified on the right.

FIG. 12g shows Atg5^{-/-} and Atg5^{-/-} MEFs that were stimulated by TNFα (1 ng/ml) and 5z7 (0.5 μM) for 8 h in the presence or absence of Apt-1 (10 μM). Cell survival was determined by CellTiter-Glo assay. Mean ±s.d. from technical quadruplicates (n=4), representative of 3 independent experiments. Two-way ANOVA, post hoc Bonferroni's tests. ***P<0.001; n.s. not significant, (P=0.2568, 0.0822 (left to right)).

FIG. 12h shows HEK293T cells that were transfected with indicated expression plasmids for 24 h. The whole-cell lysate lysed in 6 M urea was subjected to pull-down with Nia'⁰ beads and analyzed by immunoblotting with anti-Beclin 1 antibody to detect ubiquitylated Beclin 1. The ubiquitination of Beclin 1 by cIAP1 was reduced upon overexpression of TRADD, which was restored by Apt-1.

FIG. 13a shows MEFs were stimulated by mTNFα (10 ng/ml) in the presence of vehicle or ICCB-19 (10 μM) for indicated time. NF-xB and MAPKs activity were determined by immunoblotting using indicated abs.

FIG. 13b shows MEFs that were stimulated by mTNFα (10 ng/ml) in the presence of vehicle or ICCB-19 (10 μM) for indicated time. The protein levels of iNOS and Cox2 were determined by immunoblotting.

FIG. 13c shows BV2 cells (a microglial-like cell line) were treated with IFNγ (1 unit/μl) for indicated time. The mRNA levels of TNFα were determined using quantitative PCR. Mean ±s.d. from technical triplicates (n=3), representative of 3 independent experiments.

FIG. 13d shows BV2 cells (a microglial-like cell line) were treated with MDP (ligand for NOD2/RIPK2 pathway) (10 μg/ml) for indicated time. The mRNA levels of TNFα were determined using quantitative PCR. Mean ±s.d. from technical triplicates (n=3), representative of 3 independent experiments.

FIG. 13e shows BV2 cells (a microglial-like cell line) were treated with Pam3CSK4 (ligand for TLR2) (10 ng/ml) for indicated time. The mRNA levels of TNFα were determined using quantitative PCR. Mean ±s.d. from technical triplicates (n=3), representative of 3 independent experiments.

FIG. 13f shows BMDMs (bone marrow-derived macrophages) were treated with LPS (ligand for TLR4) (10 ng/ml) for indicated time. The mRNA levels of TNFα were determined using quantitative PCR. Mean ±s.d. from technical triplicates (n=3), representative of 3 independent experiments.

FIG. 13g shows BMDMs (bone marrow-derived macrophages) were treated with MDP (ligand for NOD2/RIPK2 pathway) (\] 10 μg/ml) for indicated time. The mRNA levels of TNFα were determined using quantitative PCR. Mean ±s.d. from technical triplicates (n =3), representative of 3 independent experiments.

FIG. 13h shows BV2 cells that were treated with IFNγ (1 unit/μl) together with Apt-1 (10 μM) or Nec-1s (10 μM) for 24 h. TNFα production was determined by ELISA.

FIG. 13i shows BV2 cells that were pretreated with Apt-1 (10 μM) or Nec-1s (10 μM) for 1 h and then MDP (10 μg/ml) was added to cells together with transfection reagent for 7 h. TNFα production was determined by ELISA.

FIG. 13j shows BV2 cells that were pretreated with Apt-1 (10 μM) or Nec-1s (10 μM) and then treated with Pam3CSK4 (10 ng/ml) for 8 h. TNFα production was determined by ELISA.

FIG. 13k shows BMDMs that were pretreated with Apt-1 (10 μM) or Nec-1s (10 μM) for 1 h and then treated with LPS (10 ng/ml) for 7 h. TNFα production was determined by ELISA.

FIG. 13l shows BMDMs that were first primed with IFNγ (10 ng/ml) for 2 h and then removed. The cells were then treated with Apt-1 (10 μM) or Nec-1s (10 μM) for 1 h and MDP (10 μg/ml) was added to cells directly and treated for 8 h. TNFα production was determined by ELISA. Mean ±s.d. from technical triplicates (n=3), representative of 3 independent experiments.

FIG. 13m shows BMDMs that were treated with LPS (10 ng/ml) in the presence of vehicle control or Apt-1 (10 μM) or Nec-1s (10 μM) for indicated time. NF-xB and MAPKs activity were determined by immunoblotting using indicated abs.

FIG. 13n shows that body temperature was measured in male mice (n=10, 8 weeks) injected with mTNFα (9.5 μg, i.v.) after pretreatment with Apt-1 (20 mg/kg) 30 min before. Control mice (n=9) received an equal amount of vehicle before mTNFα challenge. Mean s.e.m. Two-way ANOVA. ***P=0.0007.

FIG. 13o shows the Kaplan Meier Survival Curve measured on mice treated as in FIG. 13n. log-rank (Mantel-Cox) test. ***P<0.001.

FIG. 14a shows parallel wells of PC12/Htt-Q103 cells that were cultured with Vehicle, ICCB-19 (10 μM), ICCB-19i (10 μM), Apt-1 (10 μM), Nec-1s (10 μM), and zVAD (20 μM) as indicated prior to the addition of Ponasterone A (5 μM) for 48 h. Nuclei were labeled with DAPI. The amount of Htt-Q103-EGFP aggregates per mm² was quantified using ImageJ. Scale bar is 100 μm. Cell viability was measured by CellTiter-Glo assay. Survival rate is compared with vehicle-treated cells. Mean ±s.d. from technical triplicates (n=3), representative of 3 independent experiments. One-way ANOVA, post hoc Dunnett's tests. ***P<0.001; n.s. not significant, (P=0.8556, 0.9195, 0.8613, 0.5687, 0.5304, 0.9998, 0.6029, 0.2821 (left to right)).

FIG. 14b shows parallel wells of PC12/Htt-Q103 cells were cultured with Vehicle, ICCB-19 (10 μM), ICCB-19i (10 μM), Apt-1 (10 μM), Nec-1s (10 μM), and zVAD (20 μM) as indicated prior to the addition of Ponasterone A (5 μM) for 48 h. Nuclei were labeled with DAPI. The amount of Htt-Q103-EGFP aggregates per mm² was quantified using ImageJ. Scale bar is 100 μm. Cell viability was measured by CellTiter-Glo assay. Survival rate is compared with vehicle-treated cells. Mean ±s.d. from technical quadruplicates (n=4). One-way ANOVA, post hoc Dunnett's tests. ***P<0.001; n.s. not significant, (P=0.8556, 0.9195, 0.8613, 0.5687, 0.5304, 0.9998, 0.6029, 0.2821 (left to right)).

FIG. 14c shows SH-SY5Y cells that were transfected with expression vectors for RFP-α-Synuclein WT, E46K, or A53T for 24 h and then treated with vehicle or Apt-1 (10 μM) for 24 h. RFP-α-Synuclein was quantified by Fluorescence/Cell (RLU) by ImageJ.

FIG. 14d shows immunoblotting of the cells treated as in FIG. 14c that were lysed and analyzed by immunoblotting for the levels of α-Synuclein. Mean ±s.d. from technical triplicates (n=3), representative of 3 independent experiments. Two-way ANOVA, post hoc Bonferroni's tests. ***P<0.001; *P=0.0262, 0.0367 (left to right).

FIG. 14e shows a quantification of GFP-Tau fluorescence (e) or immunoblots of tau levels (f) in H4 cells treated with Apt-1. Mean ±s.d. from technical triplicates (n=3), representative of 3 independent experiments. Two-way ANOVA, post hoc Bonferroni's tests.

FIG. 14f shows immunoblots of tau levels in H4 cells treated with Apt-1. Mean ±s.d. from technical triplicates (n=3), representative of 3 independent experiments. Two-way ANOVA, post hoc Bonferroni's tests.

FIG. 14g contains epresentative images and quantification of cultured brain slices from PS19 mice (4 months old) stained with phospho-tau (red) and cell nuclei (DAPI/blue). Mean ±s.e.m. from biological replicates (n=5). Two-tailed t-test.

FIG. 14h shows immunoblots of tau levels in cultured PS19 mouse (4 months old) brain slices treated with indicated compounds.

FIG. 14i shows the pharmacokinetics of Apt-1 over 24 h dosing period in cerebrospinal fluid (CSF) and hippocampus. Apt-1 was delivered using intracerebroventricular Alzet micro-osmotic pump (20 mM Apt-1, 100 l, release rate: 0.25 l/h). CSF was collected at 1 h, 6 h, and 24 h. Hippocampi were collected at 24 h. The concentrations of Apt-1 were measured by HPLC. The concentration of Apt-1 in hippocampus at 24 h was 6.27 μM. Mean ±s.e.m. (n=3 mice in each group).

FIG. 14j shows synthetic preformed fibrils (pffs) [5 g full length tau (2N4R) with P301S mutation (T40/PS) per injection] or vehicle were injected into the hippocampi of PS19 mice (8 weeks old). Three weeks after the pffs injection, Apt-1 was delivered intracerebroventrically by Alzet micro-osmotic pumps (20 mM Apt-1, release rate 0.25 μl/h) for one week before sacrificing. The hippocampi were isolated from the mice for immunoblotting using TAU-5 (Thermo Fisher).

FIG. 14k shows the immunostaining for phospho-Tau (AT8) from the fibrils of FIG. 14j. Dots represent the mean from individual mice. Mean±s.e.m. (n=3 mice in each group). Two-tailed t-test. ***P=0.0007.

FIG. 15a shows expression constructs encoding Flag-TRADD-N(1-179) and HA-TRADD-C(180-312) were transfected into HEK293T cells for 20 h. Then the cells were treated with Apt-1 (10 μM) or vehicle for another 4 h. The binding between Flag-TRADD-N (1-179) and HA-TRADD-C(180-312) was analyzed by Co-IP assay as indicated.

FIG. 15b shows the effect of Apt-1 and ICCB-19 on the binding between TRADD-N and TRADD-C was determined by NanoBiT assay. Constructs were made encoding LgBiT and SmBiT fused to the N and C termini of TRADD-N and TRADD-C, respectively. HEK293T cells were transfected with these two plasmids for 24 h and then treated with indicated compounds for 4 h. The luminescence indicating the interaction of TRADD-N and TRADD-C was measured using Nano-Glo Live Cell Reagent. Mean ±s.d. from quintuplicates (n=5).

FIG. 15c shows the same effect as is FIG. 15b. Mean ±s.d. from quadruplicates (n=4).

FIG. 15d shows the same effect as is FIG. 15b. Mean ±s.d. from sextuplicates (n=6) (d). One-way ANOVA, post hoc Dunnett's test. ***P<0.001.

FIG. 15e shows a test of the Nano-Bit system assay using a known binding pair: PRKAR2A and PRKACA. Mean ±s.d. from technical triplicates (n=3), representative of 3 independent experiments.

FIG. 15f shows that Apt-1 (10 μM) does not affect the Nano-Bit system assay as determined by using the known binding pair: PRKAR2A and PRKACA. Mean ±s.d. from technical triplicates (n=3), representative of 3 independent experiments.

FIG. 15g shows that Apt-1 reduces the binding between TRADD-N and TRAF2-C in a dose-dependent manner as determined by NanoBiT assay. Mean ±s.d. from quadruplicates (n=4).

FIG. 15h depicts a schematic representation of a cell-free Forster resonance energy transfer (FRET)-based assay to detect TRADDN and TRAF2C interaction.

FIG. 15i shows the purification of indicated proteins for FRET assay expressed in HEK293T cells. Proteins were pulled down by anti-Flag affinity gel and eluted by 3 XFlag peptide. CBB staining of the proteins are shown on the right.

FIG. 15j shows a FRET-based assay to measure the direct interaction of TRADD-N and TRAF2-C was developed in which the donor Flag-TRAF2C-mCenulean (TRAF2C-mC) was excited at 430 nm, and the emission was measured from 450 to 600 nm. When FRET occurs, the acceptor mVenus-TRADDN-Flag (mV-TRADDN) emission will increase and the donor emission will decrease. Apt-1 was added to the system with indicated concentration and incubated for 1 h, then subjected to FRET assay.

FIG. 15k shows the effect of TRAF2 on the binding between TRADD-N and TRADD-C determined by NanoBiT assay. HEK293T cells were transfected with the plasmids as indicated for 24 h and then treated with Apt-1 (10 μM) for 4 h, then luminescence was measured. Mean ±s.d. from technical sextuplicates (n=6), representative of 3 independent experiments. One-way ANOVA, post hoc Dunnett's test. ***P<0.001.

FIG. 15l shows U937 cells were stimulated with TNFα (10 ng/ml) for indicated minutes in the presence of vehicle or Apt-1 (10 μM) and the complex I was pulled down using anti-TNFR1. Levels of cIAP1, TRAF2, and TRADD recruitment were determined by immunoblotting. Quantifications of cIAP1, TRAF2, and TRADD are shown below each blot.

FIG. 15m shows that due to the lack of a good anti-TRADD antibody for immunoprecipitation, Tradd^{-/-} MEFs were reconstituted with Flag-mTRADD. Cells were treated with indicated concentrations of Apt-1 for 12 h, then co-IP was performed using anti-Flag antibody followed by immunoblotting using indicated antibodies.

FIG. 16a shows a fluorescence-based thermal shift assay that was developed to quantify ICCB-19/Apt-1 binding to TRADD by measuring changes in thermal denaturation temperature (Tm). CBB staining of GST-tag and GST-TRADD purified from HEK293T cells is shown.

FIG. 16b shows in vitro binding of GST-TRADD (50 μM) with ICCB-19 (250 μM) and Apt-1 (250 μM) was determined by thermal shift assay. Thermal unfolding of GST-TRADD is monitored using SYPRO Orange. Data were collected in the presence of ICCB-19 and Apt-1, leading to a rightward shift in the unfolding transition. The apparent melting temperature (Tm) is the peak in the derivative of the unfolding curve (dF/dT), which is used as an indicator of thermal stability.

FIG. 16c shows the GST-tag (50 μM) does not bind to the compounds (250 μM) as determined by thermal shift assay.

FIG. 16d shows that ICCB-19i does not bind to GST-TRADD as determined by thermal shift assay.

FIG. 16e shows that GST-TRADD-C(50 μM) alone does not bind to either ICCB-19 (250 μM) or Apt-1 (250 μM) as determined by thermal shift assay.

FIG. 16f shows f-h, TRADD-N/ICCB-19 (f), TRADD-N/Apt-1 (g), and TRADD-N/ICCB-19i (h) samples used for STD NMR experiments were prepared as 1 mM ICCB-19 (f), 1 mM Apt-1 (g), 1 mM ICCB-19i (h), and 13 μM TRADD-N in 0.5 mL of PBS in D₂O (10%). The on-resonance irradiation of TRADD-N was performed at a chemical shift of −0.5 ppm, whereas the off-resonance irradiation was conducted at 37 ppm. Spectra were acquired using the following parameters: spectral window of 6.4 kHz, number of scans at 320, acquisition time of 2 s, and repetition time of 3 s. The decrease in signal intensity in STD spectrum, resulting from the transfer of saturation from the protein to the ligand, is evaluated by subtracting the on-resonance spectrum from the off-resonance spectrum. This subtraction yields a positive signal from a bound ligand. The asterisks indicate the signals of the compounds. The results of The STD data suggest that both ICCB-19/Apt-1, but not ICCB-19i, bind with TRADD-N.

FIG. 16i shows CBB staining of 6×His- and Flag-tagged TRADD for SPR purified from HEK293T cells. The proteins were pulled down by anti-Flag affinity gel and eluted by 3 XFlag peptide. The proteins were further purified by size exclusion chromatograph on a Superdex 75 column (GE Healthcare) in a buffer containing 20 mM imidazole (pH 6.6), 200 mM NaCl, 20 mM DTT.

FIG. 16j shows BIAcore SPR analysis of ICCB-19 binding to TRADD-N. The kinetic profile of ICCB-19 binding to TRADD-N is shown. A series of concentrations of ICCB-19 (ranging from 0.3125 to 10 μM) was used to measure the binding kinetics, with TRADD-N immobilized on the CM5 chip.

FIG. 17a shows a superposition of 2D ¹H-¹⁵N HSQC spectra of ¹⁵N-labeled His-TRADD-N(250 μM) in the presence (red) and absence (blue) of Apt-1 (500 μM).

FIG. 17b shows a superposition of 2D ¹H-¹⁵N HSQC spectra of ¹⁵N-labeled His-TRADD-N(250 μM) in the presence (red) and absence (blue) of ICCB-19 (500 μM).

FIG. 17c shows a superposition of 2D ¹H-¹⁵N HSQC spectra of ¹⁵N-labeled His-TRADD-N(250 μM) in the presence (red) and absence (blue) of ICCB-19i (500 μM). The close-up view of the region exhibited large perturbations was shown right.

FIG. 17d shows the binding pose of ICCB-19 in complex with TRADD-N was generated by induced-fit docking. The left panel demonstrated the shape and polarity of the ligand binding pocket surface, with red regions indicating negatively charged and blue positively charged. The right panel showed details of the interactions between the compound and TRADD-N. The compound was shown as cyan sticks, and the protein was shown as pink cartoon with key residues highlighted in sticks. Hydrogen bonds were shown as red dashed lines.

FIG. 17e shows the Coomassie blue staining of WT and each mutant protein for thermal shift assay.

FIG. 17f shows HEK293T cells were seeded at 7.5×10′ cells per well in a white, clear-bottom 96-well plate 24 h before transfection. Cells were then transfected with the indicated plasmids for 24 h. Medium was removed and replaced with Opti-MEM medium (100 μl) for 1 h at 37° C. The Nano-Glo reagent was prepared and added to each well immediately before the luminescence reading was taken. Luminescence was measured immediately on a plate reader and reported as relative light units (RLU). Mean ±s.d. from technical sextuplicates (n =6), representative of 3 independent experiments.

FIG. 17g shows HEK293T cells were seeded at 7.5×10′ cells per well in a white, clear-bottom 96-well plate 24 h before transfection. Cells were then transfected with the indicated plasmids for 24 h. Medium was removed and replaced with Opti-MEM medium (100 μl) for 1 h at 37° C. The Nano-Glo reagent was prepared and added to each well immediately before the luminescence reading was taken. Luminescence was measured immediately on a plate reader and reported as relative light units (RLU). Mean ±s.d. from technical sextuplicates (n =6), representative of 3 independent experiments.

FIG. 18a shows Tradd^{-/-} MEFs that were reconstituted with Flag-tagged WT or mutant TRADD as indicated. Expression levels of TRADD were determined by immunoblotting.

FIG. 18b shows Tradd^{-/-} MEFs transfected with Flag-tagged WT or indicated TRADD mutants that were stimulated by TNFα/5z7 for 9 h in the presence or absence of Apt-1 (10 M). Cell survival was determined by CellTiter-Glo assay. Mean ±s.d. from technical triplicates (n=3), representative of 3 independent experiments. Two-tailed t-test.

FIG. 18c shows TRADD-N(G121A)/Apt-1 samples for STD-NMR analyses were prepared as that of WT TRADD in FIG. 12f with 1 mM Apt-1 and 13 μM TRADD-N(G121A) in 0.5 mL of PBS in D₂O (10%).

FIG. 18d shows TRADD-N(G121A)/ICCB-19 samples for STD-NMR analyses were prepared as that of WT TRADD in FIG. 12f with 1 mM ICCB-19 and 13 μM TRADD-N(G121A) in 0.5 mL of PBS in D₂O (10%).

FIG. 18e shows the BIAcore SPR analysis of Apt-1 binding to TRADD-N(G121A). The kinetic profile of Apt-1 binding to TRADD-N(G121A) is shown. A series of concentrations of Apt-1 (ranging from 0.15625 to 5 μM) was used to measure the binding kinetics, with TRADD-N(G121A) immobilized on the CM5 chip.

FIG. 18f shows Tradd^{-/-} MEFs that were reconstituted with Flag-tagged WT or indicated TRADD mutants. The expression levels of TRADD were determined by immunoblotting.

FIG. 18g shows Tradd^{-/-} MEFs reconstituted with Flag-mutant TRADD (Y16A/F18A or Y16A/I72A/R119A) that were stimulated by TNFα/5z7 for indicated time in the presence or absence of Apt-1 (10 μM). The cell survival was determined by CellTiter-Glo assay. Mean s.d. from technical triplicates (n=3), representative of 3 independent experiments.

FIG. 18h shows Tradd^{-/-} MEFs reconstituted with Flag-mutant TRADD (Y16A/F18A or Y16A/I72A/R119A) that were stimulated by TNFα/5z7 for indicated time in the presence or absence of Apt-1 (10 μM). The cell survival was determined by CellTiter-Glo assay. Mean s.d. from technical triplicates (n=3), representative of 3 independent experiments.

FIG. 19 depicts a model for mechanism by which Apt-1 targets TRADD to inhibit RDA and activate autophagy. In TNFα-stimulated cells: Apt-1 binds to TRADD-N to reduce its binding with TRADD-C which stabilizes the binding of TRADD mediated by its DD in TRADD-C with the DD in TNFR1. The binding of Apt-1 with TRADD in complex I modulates the K63/M1 ubiquitination of RIPK1 by reducing the binding of TRADD with TRAF2/cIAP1/2 which increases the recruitment of A20 and HOIP to inhibit the activation of RIPK1 kinase. Increased retention of TRADD in complex I also decreases cytosolic availability of TRADD for the formation of complex IIa in which TRADD is known to be a key component. Treatment with Apt-1 also reduces the activation of NF-κB in TNFα-stimulated cells. In autophagy pathway: TRADD normally binds to TRAF2 and cIAP1/2 homeostatically. Apt-1 can release TRAF2 and cIAP1/2 from their binding with TRADD. Released TRAF2/cIAP1/2 in turn mediates K63 ubiquitination of Beclin 1 to promote the formation of Vps34 complex, production of PtdIns3P, and activation of autophagy.

FIG. 20 contains bar graphs showing cell viability. Specifically, wild-type or TRADD knockout MEF cells were pretreated for 1 h with 10 uM of the specified compounds, followed by 2 h treatment with vehicle or 0.5 uM 5z-7-oxozeaenol and 1 ng/mL TNFα. Cell viability was assessed using CellTiter-Glo.

FIG. 21 shows that in H4 and MEF cells, ICCB-49 and ICCB-63 induce autophagy. Wild-type H4 and MEF cells that were pretreated for 1 h with 10 uM of the specified compounds, followed by 6 h treatment with either vehicle or 40 mM NH₄Cl. Cell lysates were prepared and immunoblotted for LC3-I and LC3-II. Autophagy was determined by LC3II levels using western blotting.

FIG. 22 shows that in MEF and Jurkat cells, ICCB-49 and ICCB-63 block cleaved caspase 3. Specifically, wild-type MEF cells were pretreated for 1 h with 10 uM of the specified compounds, followed by 2 h treatment with 0.5 uM 5z-7-oxozeaenol and 1 ng/mL TNFα. Cell lysates were prepared and immunoblotted for cleaved-caspase 3 (CC3). Wild-type Jurkat cells were also pretreated for 1 h with 10 uM of the specified compounds, followed by 12 h treatment with 50 nM Velcade. Cell lysates were prepared and immunoblotted for cleaved-caspase 3 (CC3).

### DETAILED DESCRIPTION

Autophagy, a cellular catabolic process, plays an important role in promoting cell survival under metabolic stress condition by mediating lysosomal-dependent turnover of intracellular constituents for recycling. Inhibition of autophagy has been proposed as a possible new cancer therapy.

### Definitions

For convenience, certain terms employed in the specification, examples, and appended claims are collected here. All definitions, as defined and used herein, supersede dictionary definitions, definitions in documents incorporated by reference, and/or ordinary meanings of the defined terms.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. When “one or more” substituents are indicated, there may be, for example, 1, 2, 3, 4 or 5 substituents.

The term “lower” when appended to any of the groups listed below indicates that the group contains less than seven carbons (i.e., six carbons or less). For example “lower alkyl” refers to an alkyl group containing 1-6 carbons.

For purposes of this invention, the chemical elements are identified in accordance with the Periodic Table of the Elements, CAS version, Handbook of Chemistry and Physics, 67th Ed., 1986-87, inside cover.

The term “alkyl” means an aliphatic or cyclic hydrocarbon radical containing from 1 to 20, 1 to 15, or 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl. The term “fluoroalkyl” means an alkyl wherein one or more hydrogens are replaced with fluorines.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “alkoxy” means an alkyl group bound to the parent moiety through an oxygen. The term “fluoroalkoxy” means a fluoroalkyl group bound to the parent moiety through an oxygen.

“Alkylthio” means an alkyl radical attached through a sulfur linking atom. “(C1-C4)-alkylthio” includes methylthio, ethylthio, propylthio, and butylthio.

The term “alkoxyalkyl” refers to an alkyl group substituted with an alkoxy group and may be represented by the general formula alkyl-O-alkyl.

The term “haloalkyl”, as used herein, refers to an alkyl group in which at least one hydrogen has been replaced with a halogen, such as fluoro, chloro, bromo, or iodo.

Exemplary haloalkyl groups include trifluoromethyl, difluoromethyl, fluoromethyl, 2-fluoroethyl, 2,2-difluoroethyl, and 2,2,2-trifluoroethyl.

“Alkenyl” means branched or straight-chain monovalent hydrocarbon radical containing at least one double bond. The substituent may specify the number of carbon atoms. Alkenyl may be mono or polyunsaturated, and may exist in the E or Z configuration. For example, “(C2-C6)alkenyl” means a radical having from 2-6 carbon atoms in a linear or branched arrangement.

The term “alkynyl”, as used herein, refers to a straight chained or branched aliphatic group containing at least one triple bond. Typically, an alkenyl group has from 2 to about 20 carbon atoms, preferably from 2 to about 10, more preferably from 2-6 or 2-4. unless otherwise defined. The term “alkynyl” is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

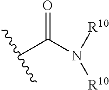

wherein each R¹⁰ independently represents a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

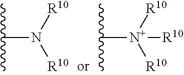

wherein each R¹⁰ independently represents a hydrogen or a hydrocarbyl group, or two R¹⁰ are taken together with the N atom to which they are attached complete a heterocycle having from 4 to 8 atoms in the ring structure. The term “aminoalkyl”, as used herein, refers to an alkyl group substituted with an amino group.

The term “aryl” as used herein include substituted or unsubstituted single-ring aromatic groups in which each atom of the ring is carbon. Preferably, the ring is a 6- to 10-membered ring, such as a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include phenyl, naphthalenyl, fluorenyl, indenyl, azulenyl, and anthracenyl.

“Cycloalkyl” means a saturated aliphatic cyclic hydrocarbon radical. The substituent may specify the number of carbon atoms. It can be monocyclic, bicyclic, polycyclic (e.g., tricyclic), fused, bridged, or spiro. For example, monocyclic (C3-C8)cycloalkyl means a radical having from 3-8 carbon atoms arranged in a monocyclic ring. Monocyclic (C3-C8)cycloalkyl includes but is not limited to cyclopropyl, cyclobutyl, cyclopentyl, cyclohexyl, cycloheptyl and cyclooctane.

A “cycloalkenyl” group is a cyclic hydrocarbon containing one or more double bonds. The cycloalkenyl ring may have 3 to 10 carbon atoms, such as 4 to 9 carbon atoms. As such, cycloalkenyl groups can be monocyclic or multicyclic. Individual rings of such multicyclic cycloalkenyl groups can have different connectivities, e.g., fused, bridged, spiro, etc. in addition to covalent bond substitution. Exemplary cycloalkenyl groups include cyclopropenyl, cyclobutenyl, cyclopentyl, cyclohexenyl, cycloheptenyl, 1,3-cyclohexadienyl, 1,4-cyclohexadienyl and 1,5-cyclooctadienyl.

Monocyclic ring systems have a single ring structure. They include saturated or unsaturated aliphatic cyclic hydrocarbon rings or aromatic hydrocarbon ring, and may specify the number of carbon atoms. The monocyclic ring system can optionally contain 1 to 3 heteroatoms in the ring structure and each heteroatom is independently selected from the group consisting O, N and S. When the heteroatom is N, it can be substituted with H, alkyl, cycloalkyl, cycloalkylalkyl, aryl, arylalkyl, heteroaryl, heteroarylalkyl (preferably, H, (C1-C6)alkyl, halo(C1-C6)alkyl or (C1-C3)alkylcarbonyl), each of which can be optionally substituted with halogen, hydroxy, alkoxy, haloalkyl, alkyl, etc. When the heteroatom is S, it can be optionally mono- or di-oxygenated (i.e. —S(O)— or S(O)₂). Examples of monocyclic ring system include, but are not limited to, cyclopropyl, cyclobutyl, cyclopentyl, cyclohexyl, cycloheptyl, cyclooctane, azetidine, pyrrolidine, piperidine, piperazine, hexahydropyrimidine, tetrahydrofuran, tetrahydropyran, oxepane, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine, tetrahydro-2H-1,2-thiazine 1,1-dioxide, and isothiazolidine 1,1-dioxide, tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, isothiazolidine 1,1-dioxide, pyrrolidin-2-one, piperidin-2-one, piperazin-2-one, morpholin-2-one, phenyl, furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole.

Bicyclic ring systems have two rings that have at least one ring atom in common. Bicyclic ring systems include fused, bridged and spiro ring systems. The two rings can both be aliphatic (e.g., cycloalkyl or heterocycloalkyl), both be aromatic (e.g., aryl or heteroaryl), or a combination thereof. The bicyclic ring systems can optionally contain 1 to 3 heteroatoms in the ring structure and each heteroatom is independently selected from the group consisting O, N and S. When the heteroatom is N, it can be substituted with H, alkyl, cycloalkyl, cycloalkylalkyl, aryl, arylalkyl, heteroaryl, heteroarylalkyl (preferably, H, (C1-C6)alkyl, halo(C1-C6)alkyl or (C1-C3)alkylcarbonyl), each of which can be optionally substituted with halogen, hydroxy, alkoxy, haloalkyl, alkyl, etc. When the heteroatom is S, it can be optionally mono- or di-oxygenated (i.e. —S(O)— or S(O)₂).

A fused bicyclic ring system has two rings which have two adjacent ring atoms in common. The two rings can both be aliphatic (e.g., cycloalkyl or heterocycloalkyl), both be aromatic (e.g., aryl or heteroaryl), or a combination thereof. For example, the first ring can be monocyclic cycloalkyl or monocyclic heterocycloalkyl, and the second ring can be cycloalkyl, partially unsaturated carbocycle, aryl, heteroaryl or a monocyclic heterocycloalkyl. For example, the second ring can be a (C3-C6)cycloalkyl, such as cyclopropyl, cyclobutyl, cyclopentyl and cyclohexyl. Alternatively, the second ring can be an aryl ring, e.g., phenyl. Examples of fused bicyclic ring systems include, but not limited to, 6,7,8,9-tetrahydro-5H-benzo[7]annulene, 2,3-dihydro-1H-indene, octahydro-1H-indene, tetrahydronaphthalene, decahydronaphthalene, indoline, isoindoline, 2,3-dihydro-1H-benzo[d]imidazole, 2,3-dihydrobenzo[d]oxazole, 2,3-dihydrobenzo[d]thiazole, octahydrobenzo[d]oxazole, octahydro-1H-benzo[d]imidazole, octahydrobenzo[d]thiazole, octahydrocyclopenta[c]pyrrole, 3-azabicyclo[3.1.0]hexane, 3-azabicyclo[3.2.0]heptane, 5,6,7,8-tetrahydroquinoline and 5,6,7,8-tetrahydroisoquinoline and 2,3,4,5-tetrahydrobenzo[b]oxepine.

Polycyclic ring systems have at least two rings, which that have at least one ring atom in common. Polycyclic ring systems include fused, bridged and spiro ring systems. The two rings can both be aliphatic (e.g., cycloalkyl or heterocycloalkyl), both be aromatic (e.g., aryl or heteroaryl), or a combination thereof. In the compounds of formula I or II, the polycyclic ring system includes a nitrogen atom (i.e., the N atom of -NR3R4). The polycyclic ring systems can optionally contain 1 to 3 additional heteroatoms in the ring structure and each heteroatom is independently selected from the group consisting O, N and S. When the heteroatom is N, it can be substituted with H, alkyl, cycloalkyl, cycloalkylalkyl, aryl, arylalkyl, heteroaryl, heteroarylalkyl (preferably, H, (C1-C6)alkyl, halo(C1-C6)alkyl or (C1-C3)alkylcarbonyl), each of which can be optionally substituted with halogen, hydroxy, alkoxy, haloalkyl, alkyl, etc. When the heteroatom is S, it can be optionally mono- or di-oxygenated (i.e. —S(O)— or S(O)₂).

A fused polycyclic ring system has at least two rings which have two adjacent ring atoms in common. The rings can each be aliphatic (e.g., cycloalkyl or heterocycloalkyl), each be aromatic (e.g., aryl or heteroaryl), or a combination thereof. For example, the first ring can be monocyclic cycloalkyl or monocyclic heterocycloalkyl, and the second ring can be a cycloalkyl, partially unsaturated carbocycle, aryl, heteroaryl or a monocyclic heterocycloalkyl. For example, the second ring can be a (C3-C6)cycloalkyl, such as cyclopropyl, cyclobutyl, cyclopentyl and cyclohexyl. Alternatively, the second ring can be an aryl ring, e.g., phenyl. Examples of fused bicyclic ring systems include, but not limited to, 6,7,8,9-tetrahydro-5H-benzo[7]annulene, 2,3-dihydro-1H-indene, octahydro-1H-indene, tetrahydronaphthalene, decahydronaphthalene, indoline, isoindoline, 2,3-dihydro-1H-benzo[d]imidazole, 2,3-dihydrobenzo[d]oxazole, 2,3-dihydrobenzo[d]thiazole, octahydrobenzo[d]oxazole, octahydro-1H-benzo[d]imidazole, octahydrobenzo[d]thiazole, octahydrocyclopenta[c]pyrrole, 3-azabicyclo[3.1.0]hexane, 3-azabicyclo[3.2.0]heptane, 5,6,7,8-tetrahydroquinoline and 5,6,7,8-tetrahydroisoquinoline and 2,3,4,5-tetrahydrobenzo[b]oxepine. An exemplary fused tricyclic ring system is 2,3-dihydro-1H-phenalene.

“Heterocycloalkyl” and “heterocyclyl” mean a saturated 4-12 membered ring containing 1 to 4 heteroatoms, which may be the same or different, selected from N, O or S and optionally containing one or more double bonds. It can be monocyclic, bicyclic, tricyclic, fused, bridged, or spiro.

When the heteroatom is N, it can be substituted with H, alkyl, cycloalkyl, cycloalkylalkyl, aryl, arylalkyl, heteroaryl, heteroarylalkyl (preferably, H, (C1-C6)alkyl, halo(C1-C6)alkyl or (C1-C3)alkylcarbonyl), each of which can be optionally substituted with halogen, hydroxy, alkoxy, haloalkyl, alkyl, etc. When the heteroatom is S, it can be optionally mono- or di-oxygenated (i.e. —S(O)— or S(O)₂).

“Heterocycloalkenyl” means a cyclic hydrocarbon containing one or more double bonds and at least one heteroatom. In some embodiments, the heteroatom is selected from N, O, and S. The cycloalkenyl ring may have 3 to 10 carbon atoms, such as 4 to 9 carbon atoms.

As such, heterocycloalkenyl groups can be monocyclic or multicyclic. Individual rings of such multicyclic heterocycloalkenyl groups can have different connectivities, e.g., fused, bridged, spiro, etc. in addition to covalent bond substitution.

“Haloalkyl” and “halocycloalkyl” include mono, poly, and perhaloalkyl groups where the halogens are independently selected from fluorine, chlorine, and bromine.

“Heteroaryl” means a monovalent heteroaromatic monocyclic or polycyclic ring radical. Heteroaryl rings are 5- and 6-membered aromatic heterocyclic rings containing 1 to 4 heteroatoms independently selected from N, O, and S, and include, but are not limited to furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Halogen” or “halo” used herein refers to fluorine, chlorine, bromine, or iodine.

The term “thioalkyl”, as used herein, refers to an alkyl group substituted with a thiol group.

The term “thioether”, as used herein, is equivalent to an ether, wherein the oxygen is replaced with a sulfur.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

Certain compounds of the present invention may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

The invention also includes various isomers and mixtures thereof “Isomer” refers to compounds that have the same composition and molecular weight but differ in physical and/or chemical properties. The structural difference may be in constitution (geometric isomers) or in the ability to rotate the plane of polarized light (stereoisomers).

Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by “ [symbol] ”which represents “ [symbol] ” “ [symbol] ” or “ [symbol] ”.

“R,” “S,” “S*,” “R*,” “E,” “Z,” “cis,” and “trans,” indicate configurations relative to the molecule having unspecified stereochemistry.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the geometry of a disclosed compound is named or depicted by structure, the named or depicted geometrical isomer is at least 60%, 70%, 80%, 90%, 99% or 99.9% by weight pure relative to the other geometrical isomers.

When a disclosed compound is named or depicted by structure without indicating the stereochemistry, and the compound has at least one chiral center, it is to be understood that the name or structure encompasses one enantiomer of the compound free from the corresponding optical isomer, a racemic mixture of the compound and mixtures enriched in one enantiomer relative to its corresponding optical isomer.

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “prodrug” means a pharmacological derivative of a parent drug molecule that requires biotransformation, either spontaneous or enzymatic, within the organism to release the active drug. For example, prodrugs are variations or derivatives of the compounds of the invention that have groups cleavable under certain metabolic conditions, which when cleaved, become the compounds of the invention. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form. Prodrug forms often offer advantages of solubility, tissue compatibility, or delayed release in the mammalian organism (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability.

As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds having a carbonate, carbamate, amide or alkyl ester moiety covalently bonded to any of the above substituents disclosed herein.

A “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein.

### Apoptosis Blockers and Autophagy Inducers

Provided herein are compounds of formula I, and pharmaceutically acceptable salts thereof:

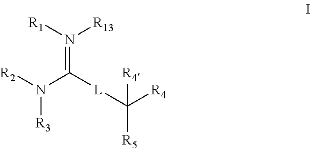

wherein
L is CH₂, NR₁ₐ, heteroaryl or S(O)n, where n is 0, 1, or 2;
R₁ₐ is independently selected from H, CN, alkyl, and aryl;
R₃ is selected from H, alkyl, and aryl;
R₄ is selected from H, alkyl, and aryl;
R_{4′} is selected from H, alkyl, and aryl;
R₅ is selected from H, alkyl, aryl, heteroaryl, —(CH₂)ₚCONR₆R₇ where p is 0, 1, or 2,
CH₂NR₆R₇, —CH(OH)NR₆R₇, —CH(OH)CH₂-cycloalkyl, —CH(OH)CH₂—NHcycloalkyl, and —CR₁₀═CHR₁₁;
   R₆ is selected from H, alkyl, C₃₋₈cycloalkyl, aryl, and -NHcycloalkyl;
   R₇ is selected from H and alkyl;
or R₆ and R₇, taken together with the nitrogen atom to which they are attached, form a heterocyclyl;
   R₁₀ is selected from H and halo;
   R₁₁ is cycloalkyl;
   R₁₃ is absent or alkyl, where the alkyl forms an iminium group; and
   (a) R₁ and R₂ are each independently selected from H, CN, alkyl, and aryl, or
   (b) R₁ and R₂, taken together with the atoms to which they are attached, form a heterocyclyl of Formula IA:

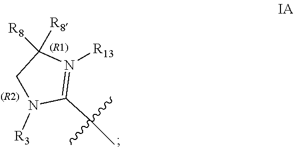

R₈ and R_{8′} are each independently selected from H, alkyl, and aryl; or
(c) R₁ and R₂, taken together with the atoms to which they are attached, and R₃ and R₄, taken together with the atoms to which they are attached, form a bicycle of Formula IB:

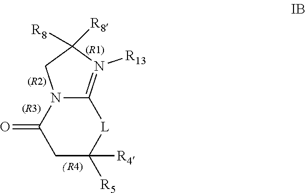

R₈ and R_{8′} are each independently selected from H, alkyl, and aryl;

further wherein when R₅ is —(CH₂)₀CONR₆R₇, then R₇ and R₄, taken together with the atoms to which they are attached, may form a heterocyclyl of Formula IC:

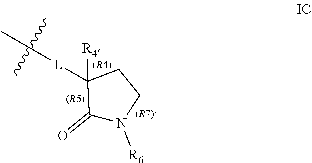

In some embodiments, the compound of Formula I is not:

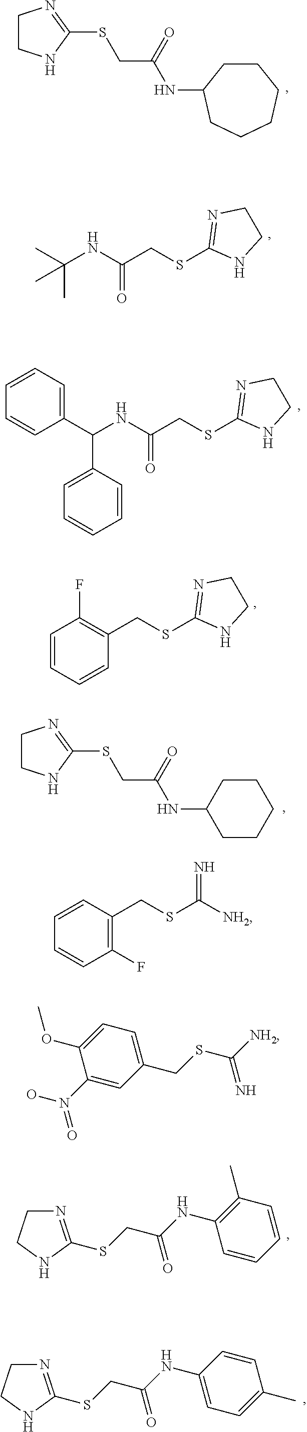

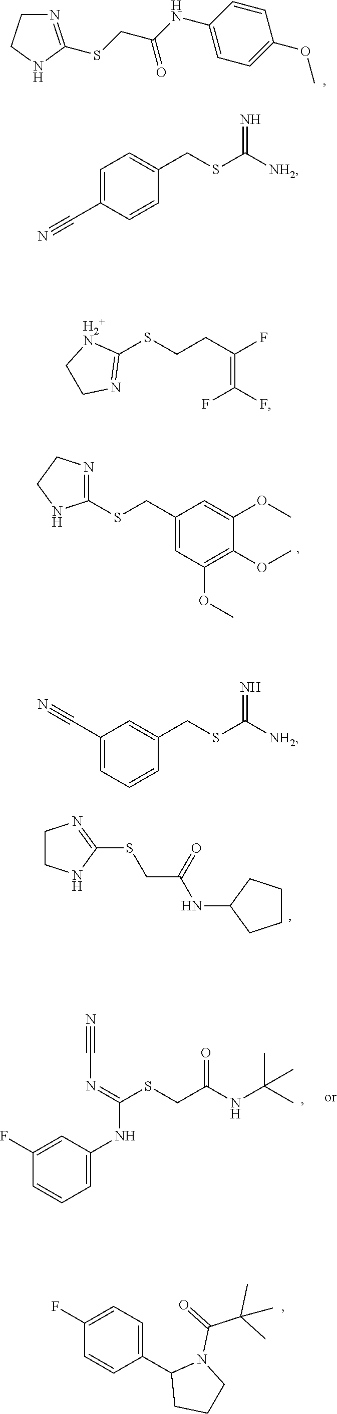

or a pharmaceutically acceptable salt of any of the foregoing.

For clarity, in Formula IA, IB, and IC, substituents that correspond to variables in Formula I are indicated in parentheses (e.g., (R₂) is shown in Formula IA at the position of R₂ in Formula I).

In some embodiments, R₁ is alkyl, such as methyl. In some such embodiments, R₂ is H or alkyl, and R₃ is H or alkyl. In certain embodiments, R₁ is alkyl, R₂ is alkyl, and R₃ is H. In other embodiments, R₁ is alkyl, R₂ is H, and R₃ is alkyl.

In some embodiments, L is S(O)n and n is 0. In certain embodiments, wherein L is S(O)n and n is 1. In other embodiments, L is S(O)n and n is 2. In certain embodiments, L is CH₂ or oxadiazolyl. In still other embodiments, L is NR₁ₐ, such as NH.

In some embodiments, R₃ is alkyl. In some such embodiments, R₃ is methyl, ethyl, or isopropyl. In other embodiments, R₃ is aryl, such as phenyl.

In some embodiments, R₃ is selected from H, methyl, ethyl, isopropyl and phenyl.

In some embodiments, R₁ and R₂, taken together with the atoms to which they are attached, form a heterocyclyl of Formula IA:

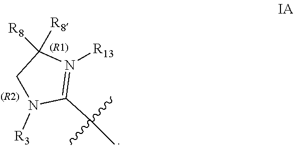

In some embodiments, R₄ is alkyl, such as methyl or isopropyl.

In other embodiments, R₄ is aryl, such as phenyl. In some such embodiments, R₄ is halo-substituted phenyl, such as chlorophenyl (e.g., 2-chlorophenyl or 4-chlorophenyl).

In still other embodiments, R₄ is H.

In certain embodiments, R₄ is selected from H, methyl, and phenyl.

In some embodiments, R_{4′} is H. In some such embodiments, R₄ is H and R_{4′} is H. In other embodiments, R_{4′} is alkyl, such as methyl.

In some embodiments, R₄ is alkyl, such as methyl, and R_{4′} is alkyl, such as methyl.

In some embodiments, R₁ and R₂, taken together with the atoms to which they are attached, and R₃ and R₄, taken together with the atoms to which they are attached, form a bicycle of Formula IB:

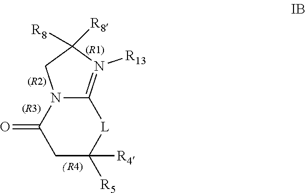

In some embodiments, R₅ is —(CH₂)ₚCONR₆R₇; and p is 0. In certain embodiments, R₅ is —(CH₂)ₚCONR₆R₇; and p is 1. In other embodiments, R₅ is —(CH₂)ₚCONR₆R₇; and p is 2. In some embodiments, R₅ is methyl or —C(O)NHC₃₋₈cycloalkyl. In certain embodiments, R₅ is selected from phenyl, pyrrolopyrimidinyl, and benzothiophenyl. In other embodiments, R₅ is unsubstituted phenyl or phenyl substituted with one or more of fluoro, chloro, methyl, methoxy, ethoxy, NO₂, or —CO₂Me.

In other embodiments, R₅ is —CR₁₀═CHR₁₁. In some such embodiments, R₁₀ is H or fluoro; and R₁ is cycloheptyl.

In some embodiments,

R₅ is —(CH₂)₀CONR₆R₇;
R₆ is unsubstituted phenyl or phenyl substituted with one or more alkyl or alkoxy groups; and

### R₇ is H.

In some embodiments,

R₅ is —(CH₂)₀CONR₆R₇;
R₆ is C₃₋₈cycloalkyl selected from cyclopropyl, cyclobutyl, cyclopentyl, cyclohexyl and cycloheptyl; and
R₇ is H or methyl.

In some embodiments, when R₅ is —(CH₂)₀CONR₆R₇, R₇ and R₄, taken together with the atoms to which they are attached, form a heterocyclyl of Formula IC:

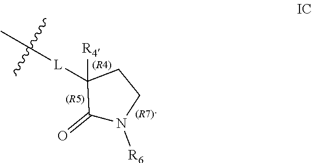

In some embodiments, R₅ is —CH(OH)CH₂-cycloalkyl, such as —CH(OH)CH₂-cycloheptyl. In other embodiments, R₅ is —CH₂NR₆R₇.

In some embodiments, R₆ is C₃₋₈cycloalkyl, such as cycloheptyl. In other embodiments, R₆ and R₇, taken together with the nitrogen atom to which they are attached, form a heterocyclyl. In still other embodiments, R₆ is alkyl, such as aralkyl. In certain such embodiments, R₆ is diphenylmethyl.

In some embodiments, R₇ is alkyl, such as methyl. In other embodiments, R₇ is H.

In some embodiments, R₈ is alkyl, such as methyl. In some such embodiments, R₈ is alkyl (such as methyl) and R_{8′} is alkyl (such as methyl). In other embodiments, R₈ is H. In other embodiments, R_{8′} is H.

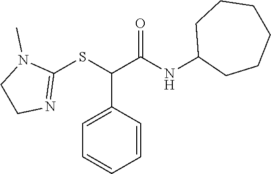 [SMILES: CN1CCN=C1SC(C(=O)NC1CCCCCC1)c1ccccc1]

In some embodiments, the compound of Formula I is (Apt-1).

Provided herein are compounds of formula II:

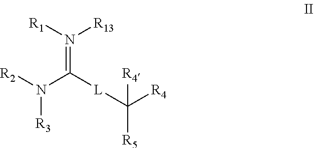

wherein

### L is NR₁ₐ or S;

R₁ₐ is independently selected from CN, alkyl, and aryl;
(a) R₁ and R₂ are each independently selected from CN, alkyl, and aryl, or
(b) R₁ and R₂, taken together with the atoms to which they are attached, form a heterocyclyl of Formula IIA:

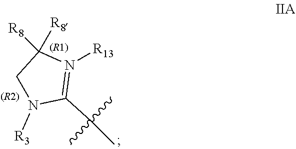

wherein R₈ and R_{8′} are each H or alkyl;
R₃ is selected from H, alkyl, and aryl;
R₄ is selected from H, alkyl, and aryl;
R_{4′} is selected from H, alkyl, and aryl;
R₅ is selected from aryl, —(CH₂)ₚCONR₆R₇ where p is 0 or 2, —CH₂NR₆R₇, —CH(OH)NR₆R₇, and —CR₁₀═CHR₁₁;
R₆ is selected from alkyl, aryl, and C₃₋₈cycloalkyl, such as C₃₋₄cycloalkyl or C₇₋₈cycloalkyl;
R₇ is selected from H and alkyl,

or R₆ and R₇, taken together with the nitrogen atom to which they are attached, form a heterocyclyl;

### R₁₀ is H;

R₁₁ is cycloalkyl;
R₁₃ is absent or alkyl, where the alkyl forms an iminium group,
or a pharmaceutically acceptable salt thereof.

In some embodiments, the compound of Formula II is not:

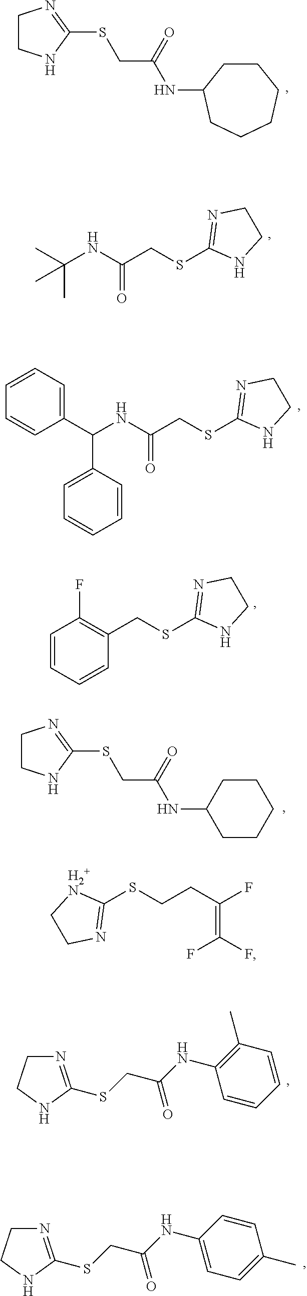

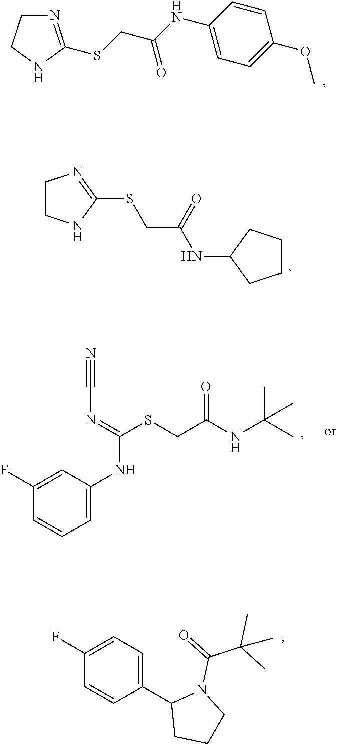 [SMILES: COc1ccc(NC(=O)CSC2=NCCN2)cc1 ; O=C(CSC1=NCCN1)NC1CCCC1 ; CC(C)(C)NC(=O)CS/C(=N\C#N)Nc1cccc(F)c1 ; CC(C)(C)C(=O)N1CCCC1c1ccc(F)cc1]

or a pharmaceutically acceptable salt of any of the foregoing.

For clarity, in Formula IIA, substituents that correspond to variables in Formula II are indicated in parentheses (e.g., (R₂) is shown in Formula IIA at the position of R₂ in Formula II).

In some embodiments, R₁ is alkyl, such as methyl. In some such embodiments, R₂ is alkyl, and R₃ is H.

In some embodiments, L is NR₁ₐ, such as NH. In other embodiments, L is S.

In some embodiments, R₃ is alkyl. In some such embodiments, R₃ is methyl, ethyl, or isopropyl. In other embodiments, R₃ is aryl, such as phenyl.

In certain embodiments, R₃ is selected from H, methyl, ethyl, isopropyl and phenyl.

In some embodiments, R₁ and R₂, taken together with the atoms to which they are attached, form a heterocyclyl of Formula IIA:

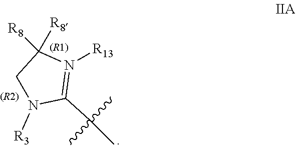

In some embodiments, R₄ is alkyl, such as methyl or isopropyl.

In other embodiments, R₄ is aryl, such as phenyl. In some such embodiments, R₄ is halo-substituted phenyl, such as chlorophenyl (e.g., 2-chlorophenyl or 4-chlorophenyl).

In still other embodiments, R₄ is H.

In certain embodiments, R₄ is selected from H, methyl, and phenyl. In certain embodiments, R₄ is aryl, such as phenyl, for example, substituted phenyl, preferably unsubstituted phenyl.

In some embodiments, R_{4′} is H. In some such embodiments, R₄ is H and R_{4′} is H. In other embodiments, R_{4′} is alkyl, such as methyl.

In some embodiments, R₄ is alkyl, such as methyl, and R_{4′} is alkyl, such as methyl.

In certain embodiments, R₅ is —(CH₂)ₚCONR₆R₇; and p is 0. In some embodiments, R₅ is —(CH₂)ₚCONR₆R₇; and p is 2.

In certain embodiments, R₆ is selected from alkyl, aryl, C₃₋₄cycloalkyl, and C₇₋₈cycloalkyl. In certain embodiments, R₆ is selected from alkyl, C₃₋₄cycloalkyl, and C₇₋₈cycloalkyl. In certain embodiments, R₆ is selected from alkyl and C₇₋₈cycloalkyl. In certain embodiments, R₆ is selected from C₃₋₄cycloalkyl and C₇₋₈cycloalkyl.

In certain embodiments, R₆ is cycloheptyl. In other embodiments, R₆ is alkyl or aryl. In other embodiments, R₆ and R₇, taken together with the nitrogen atom to which they are attached, form a heterocyclyl. In still other embodiments, R₆ is alkyl, such as aralkyl. In certain such embodiments, R₆ is diphenylmethyl.

In some embodiments, R₇ is alkyl, such as methyl.

In certain embodiments, R₇ is H or methyl. In some embodiments, R₅ is —CR₁₀═CHR₁₁; R₁₀ is H; and R₁₁ is cycloheptyl.

In some embodiments, R₈ is alkyl, such as methyl. In some such embodiments, R₈ is alkyl (such as methyl) and R_{8′} is alkyl (such as methyl). In other embodiments, R₈ is H. In other embodiments, R_{8′} is H.

In certain embodiments, the compound of formula II is not

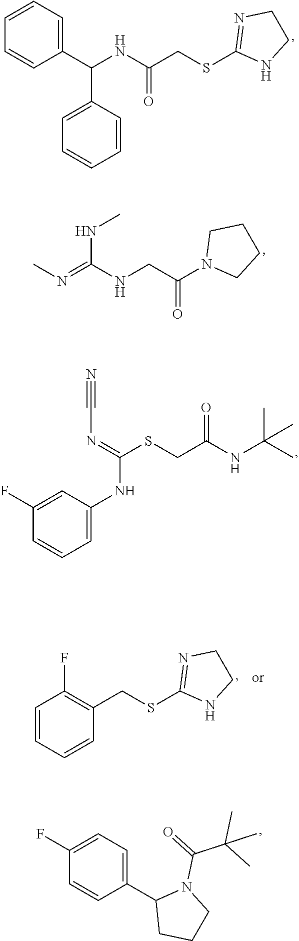 [SMILES: O=C(CSC1=NCCN1)NC(c1ccccc1)c1ccccc1 ; C/N=C(\NC)NCC(=O)N1CCCC1 ; CC(C)(C)NC(=O)CS/C(=N\C#N)Nc1cccc(F)c1 ; Fc1ccccc1CSC1=NCCN1 ; CC(C)(C)C(=O)N1CCCC1c1ccc(F)cc1]

or a pharmaceutically acceptable salt of any of the foregoing.

Provided herein are compounds selected from:

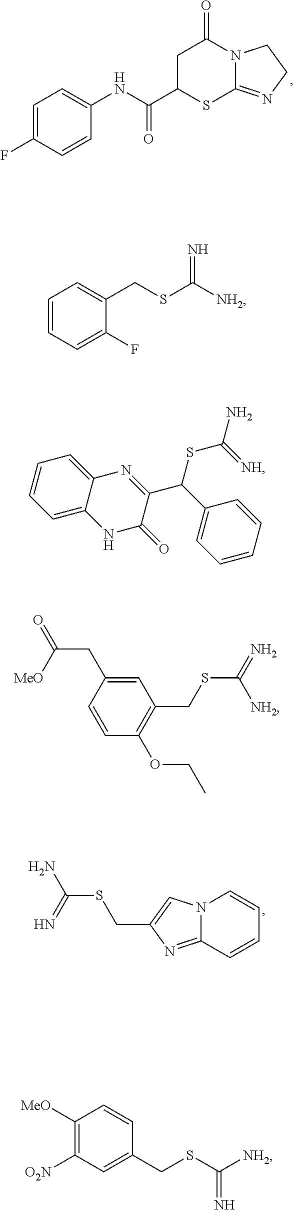 [SMILES: O=C(Nc1ccc(F)cc1)C1CC(=O)N2CCN=C2S1 ; N=C(N)SCc1ccccc1F ; N=C(N)SC(c1ccccc1)c1nc2ccccc2[nH]c1=O ; CCOc1ccc(CC(=O)OC)cc1CSC(=N)N ; N=C(N)SCc1cn2ccccc2n1 ; COc1ccc(CSC(=N)N)cc1[N+](=O)[O-]]

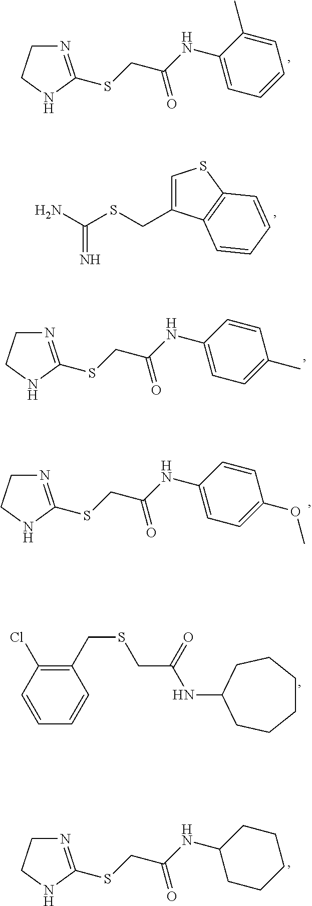 [SMILES: Cc1ccccc1NC(=O)CSC1=NCCN1 ; N=C(N)SCc1csc2ccccc12 ; Cc1ccc(NC(=O)CSC2=NCCN2)cc1 ; COc1ccc(NC(=O)CSC2=NCCN2)cc1 ; O=C(CSCc1ccccc1Cl)NC1CCCCCC1 ; O=C(CSC1=NCCN1)NC1CCCCC1]

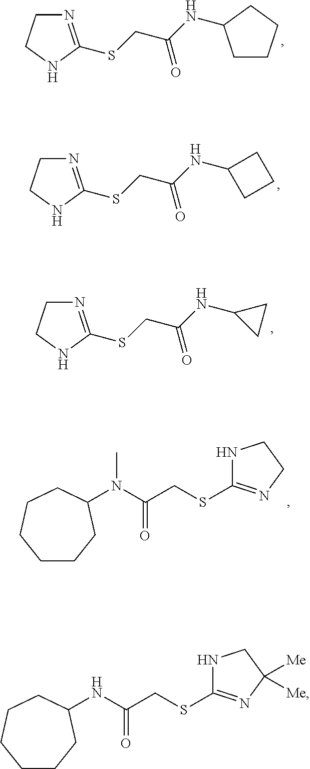 [SMILES: O=C(CSC1=NCCN1)NC1CCCC1 ; O=C(CSC1=NCCN1)NC1CCC1 ; O=C(CSC1=NCCN1)NC1CC1 ; CN(C(=O)CSC1=NCCN1)C1CCCCCC1 ; CC1(C)CNC(SCC(=O)NC2CCCCCC2)=N1]

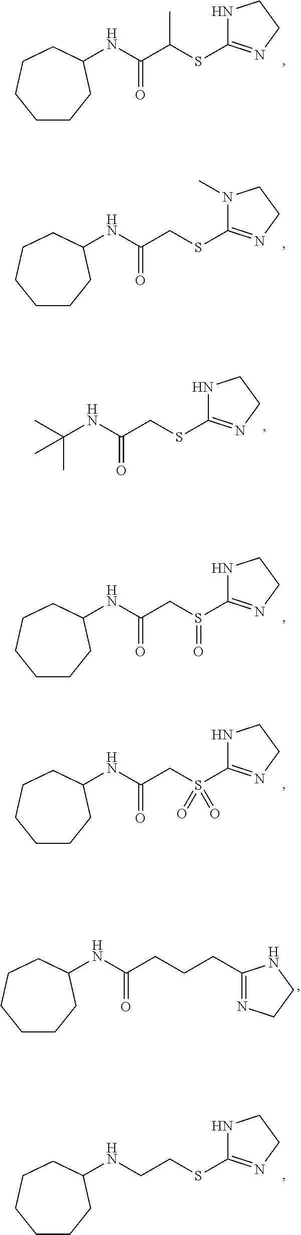

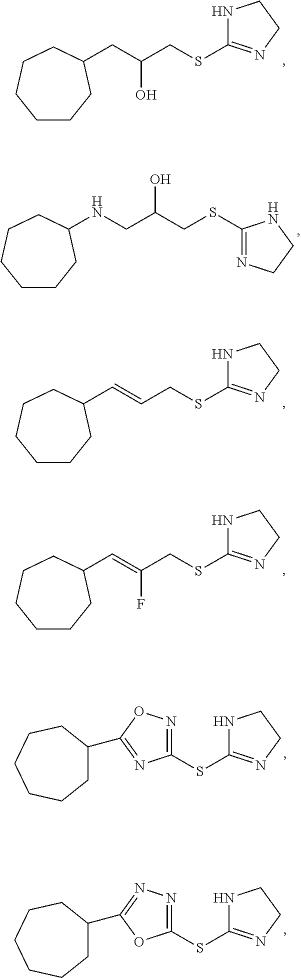 [SMILES: OC(CSC1=NCCN1)CC1CCCCCC1 ; OC(CNC1CCCCCC1)CSC1=NCCN1 ; C(=C\C1CCCCCC1)/CSC1=NCCN1 ; F/C(=C\C1CCCCCC1)CSC1=NCCN1 ; C1CCCC(c2nc(SC3=NCCN3)no2)CC1 ; C1CCCC(c2nnc(SC3=NCCN3)o2)CC1]

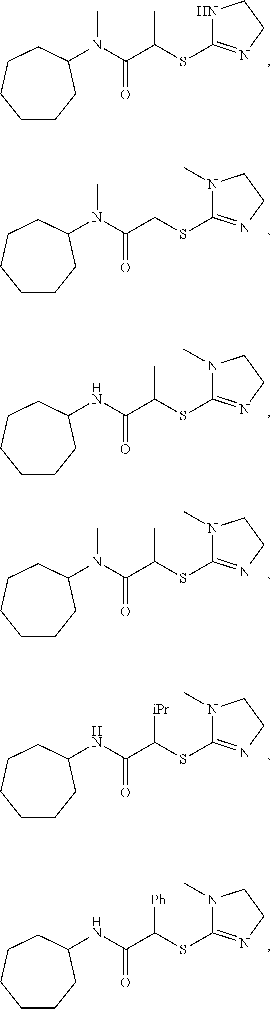 [SMILES: CC(SC1=NCCN1)C(=O)N(C)C1CCCCCC1 ; CN1CCN=C1SCC(=O)N(C)C1CCCCCC1 ; CC(SC1=NCCN1C)C(=O)NC1CCCCCC1 ; CC(SC1=NCCN1C)C(=O)N(C)C1CCCCCC1 ; CC(C)C(SC1=NCCN1C)C(=O)NC1CCCCCC1 ; CN1CCN=C1SC(C(=O)NC1CCCCCC1)c1ccccc1]

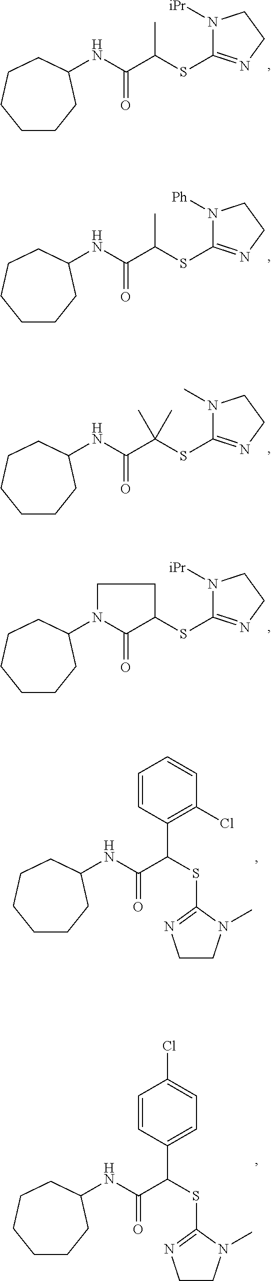 [SMILES: CC(SC1=NCCN1C(C)C)C(=O)NC1CCCCCC1 ; CC(SC1=NCCN1c1ccccc1)C(=O)NC1CCCCCC1 ; CN1CCN=C1SC(C)(C)C(=O)NC1CCCCCC1 ; CC(C)N1CCN=C1SC1CCN(C2CCCCCC2)C1=O ; CN1CCN=C1SC(C(=O)NC1CCCCCC1)c1ccccc1Cl ; CN1CCN=C1SC(C(=O)NC1CCCCCC1)c1ccc(Cl)cc1]

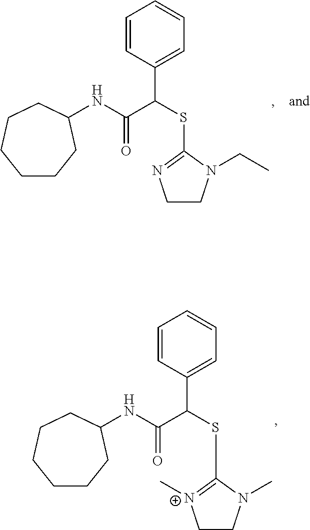 [SMILES: CCN1CCN=C1SC(C(=O)NC1CCCCCC1)c1ccccc1 ; CN1CC[N+](C)=C1SC(C(=O)NC1CCCCCC1)c1ccccc1]

and pharmaceutically acceptable salts of any of the foregoing.

Provided herein are compounds selected from:

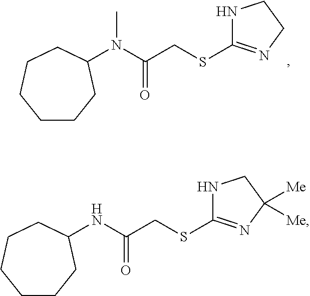 [SMILES: CN(C(=O)CSC1=NCCN1)C1CCCCCC1 ; CC1(C)CNC(SCC(=O)NC2CCCCCC2)=N1]

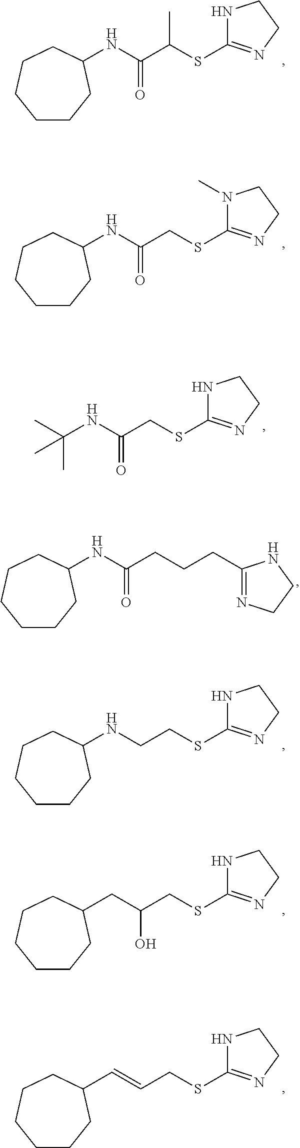

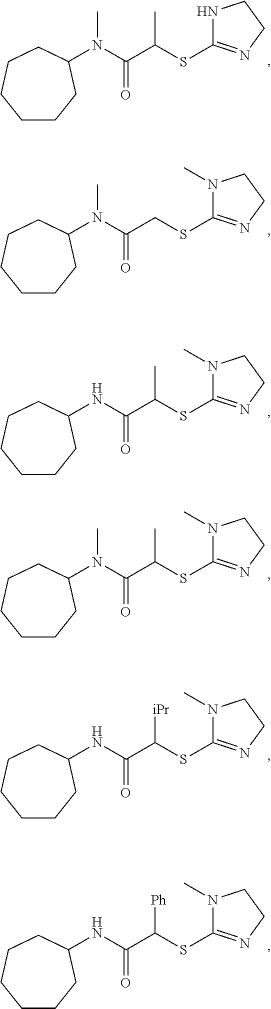 [SMILES: CC(SC1=NCCN1)C(=O)N(C)C1CCCCCC1 ; CN1CCN=C1SCC(=O)N(C)C1CCCCCC1 ; CC(SC1=NCCN1C)C(=O)NC1CCCCCC1 ; CC(SC1=NCCN1C)C(=O)N(C)C1CCCCCC1 ; CC(C)C(SC1=NCCN1C)C(=O)NC1CCCCCC1 ; CN1CCN=C1SC(C(=O)NC1CCCCCC1)c1ccccc1]

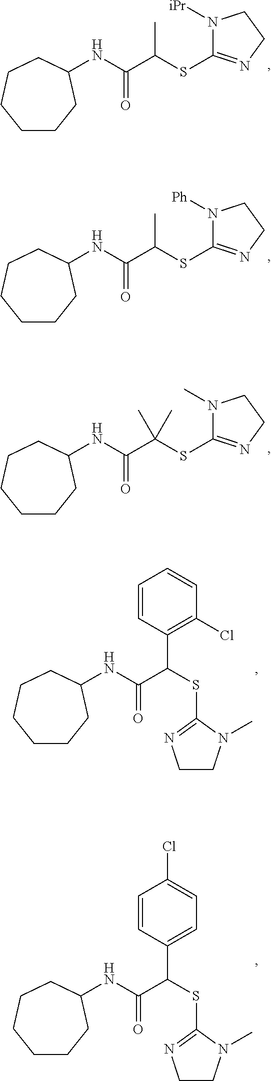 [SMILES: CC(SC1=NCCN1C(C)C)C(=O)NC1CCCCCC1 ; CC(SC1=NCCN1c1ccccc1)C(=O)NC1CCCCCC1 ; CN1CCN=C1SC(C)(C)C(=O)NC1CCCCCC1 ; CN1CCN=C1SC(C(=O)NC1CCCCCC1)c1ccccc1Cl ; CN1CCN=C1SC(C(=O)NC1CCCCCC1)c1ccc(Cl)cc1]

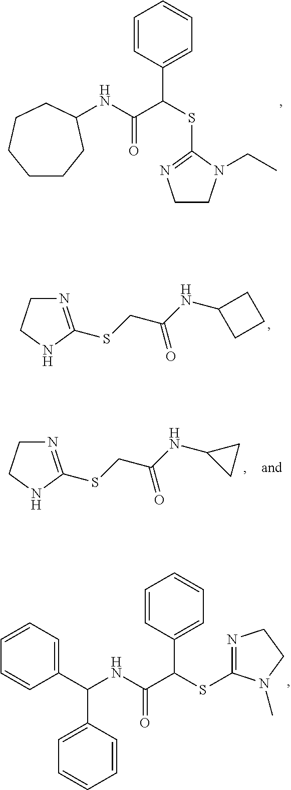 [SMILES: CCN1CCN=C1SC(C(=O)NC1CCCCCC1)c1ccccc1 ; O=C(CSC1=NCCN1)NC1CCC1 ; O=C(CSC1=NCCN1)NC1CC1 ; CN1CCN=C1SC(C(=O)NC(c1ccccc1)c1ccccc1)c1ccccc1]

and pharmaceutically acceptable salts of any of the foregoing. In some embodiments, the compound of formula II has the structure.

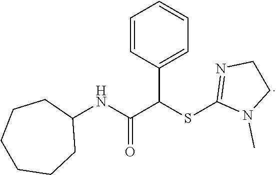 [SMILES: CN1CCN=C1SC(C(=O)NC1CCCCCC1)c1ccccc1]

Also provided herein is a compound selected from

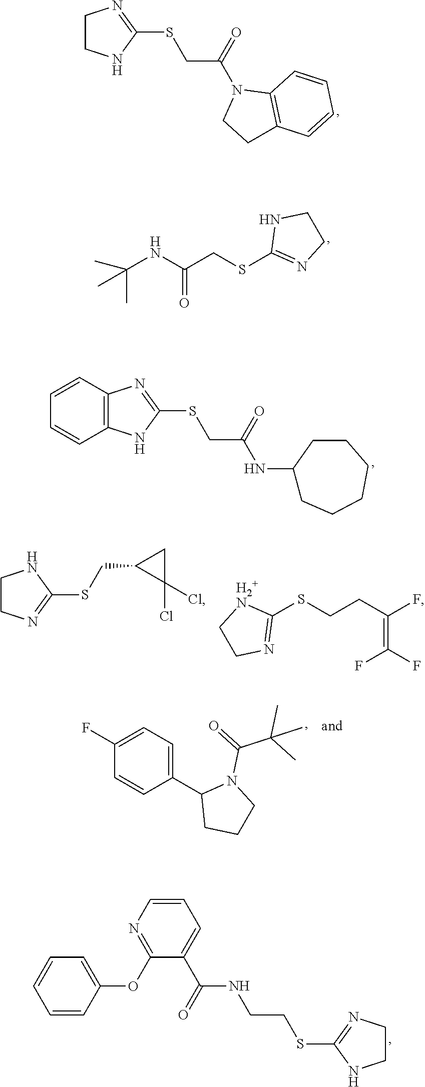

or a pharmaceutically acceptable salt thereof.

### Pharmaceutical Compositions

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. For example, one aspect of the invention relates to a pharmaceutical composition comprising a compound of formula I or II (e.g., a therapeutically effective dose of a compound of formula I or II), or a pharmaceutically acceptable salt, biologically active metabolite, solvate, hydrate, prodrug, enantiomer or stereoisomer thereof; and a pharmaceutically acceptable diluent or carrier. Another aspect of the invention relates to a pharmaceutical composition comprising a compound selected from and (e.g., a

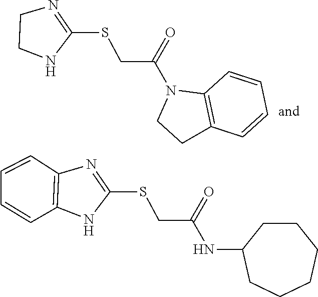 [SMILES: O=C(CSC1=NCCN1)N1CCc2ccccc21 ; O=C(CSc1nc2ccccc2[nH]1)NC1CCCCCC1]

therapeutically effective amount of a compound selected from and

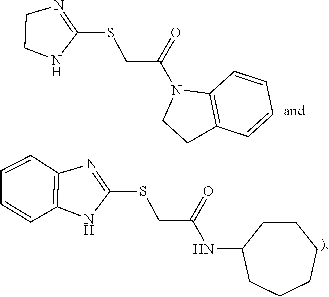 [SMILES: O=C(CSC1=NCCN1)N1CCc2ccccc21 ; O=C(CSc1nc2ccccc2[nH]1)NC1CCCCCC1]

or a pharmaceutically acceptable salt, biologically active metabolite, solvate, hydrate, prodrug, enantiomer or stereoisomer thereof; and a pharmaceutically acceptable diluent or carrier.

As used herein, a therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl-N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di alkyl-N-(hydroxy alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Methods of Use

Apoptosis is a caspase-mediated cellular suicide pathway in metazoan and can be activated to mediate acute tissue injuries and diseases such as stroke, heart attack and spinal cord injuries as well as in neurodegenerative diseases associated with aging.

Apoptosis can be activated by TNFα and other cognate ligands of the death receptor family. The stimulation of TNFR1 by TNFα triggers the rapid formation of complex I associated with the intracellular death domain (DD) of TNFR1. Two intracellular DD containing proteins, adaptor protein TRADD and a kinase RIPK1, are recruited into complex I by DD-mediated homotypic interactions with the DD of TNFR1. In complex I, TRADD recruits adaptor protein TRAF2 and E3 ubiquitin ligases cIAP1/2, which in turn modulates the ubiquitination of RIPK1 directly and also indirectly by mediating the recruitment of M1 ubiquitin ligase complex LUBAC. Ubiquitination of RIPK1 and TNFR1 collectively promotes the recruitment and activation of TBK1, TAK1 and IKK to mediate the activation of NF-κB pathway, and A20, an important E3 ubiquitin editing enzyme that can modulate the activation of RIPK1 by reducing its K63 ubiquitination.

TBK1 and TAK1 as well as the downstream kinases activated by TAK1 including IKK and MK2 are important for suppressing RIPK1 activation to block RIPK1-dependent apoptosis (RDA). TNFα stimulation of cells with deficiencies in TAK1, TBK1, and IKK promotes the formation of a downstream execution complex, complex IIa, that includes TRADD, RIPK1, FADD and caspase-8 to mediate the activation of caspase-8 and downstream caspases such as caspase-3. Aging human brains show significant reduction of TAK1, suggesting that the increased vulnerability to RDA may be involved in mediating the onset of common neurodegenerative diseases associated with aging.

Accumulation of misfolded and neurotoxic proteins is a common feature of human neurodegenerative diseases such as Alzheimer's disease, Parkinson's disease, Huntington's disease and amyotrophic lateral sclerosis. Promoting the removal of misfolded proteins is considered as the goal of potential therapeutic strategies for neurodegeneration. The activation of autophagy leads to the formation of double membraned autophagosomes which sequester large protein oligomers and aggregates that may not be degradable by proteasome.

Reduced levels of autophagy in aging brains may contribute to the onset of neurodegeneration. Thus, modulating the levels of autophagy provides a therapeutic strategy to reduce the accumulation of misfolded proteins for the treatment of neurodegenerative diseases.

Disclosed herein are compounds and compositions for removing misfolded and aggregated proteins by activating autophagy and inhibiting apoptosis, which contributes to neuronal loss in neurodegenerative diseases. Their efficacy can be measured using several methods known in the art, such as an assay using Jurkat cells to assess apoptosis levels as described herein. Compounds that effectively induce autophagy can be identified using cell assays with H4-LC3-GFP cells as described herein. Certain disclosed compounds can protect cells against apoptosis induced by proteasomal stress as well as increase the autophagy influx.

Compounds and compositions disclosed herein are also effective at blocking RDA induced by TNFα. TRADD is involved in the induction of autophagy by disclosed compounds. Certain compounds can increase the levels of autophagy in Tradd−/− MEFs compared to that of WT. Certain compounds can also inhibit the activation of RIPK1 and caspase-3 in models of RDA, e.g. Tbk1−/− MEFs and Nemo−/− MEFs treated with TNFα alone. Also, the catalytic activity of caspase-8 can be inhibited in MEFs treated with TNFα and 5z7. Other pathways, such as transcriptional regulation of cFLIP, a suppressor of caspase-8, are important mechanisms for protecting against TNFα-mediated apoptosis. Certain compounds can increase the levels of cFLIP mRNA and showed dependence on upon TRADD.

Overexpression of Tradd has been shown to promote the activation of NF-κB pathway. The N-terminal domain of TRADD interacts with TRAF2, which in turn recruits E3 ubiquitin ligases cIAP1/2, to complex I to promote the activation of NF-κB which in turn supports cell survival by mediating the expression of cFLIP. The NF-κB pathway is involved in the protection of apoptosis. Increased recruitment of TRADD in complex I in cells treated with compounds disclosed herein promotes pro-survival signaling by mediating NF-κB activation and suppressing the activation of RIPK1.

The effect of disclosed compounds in mediating inflammatory response can be evaluated by measuring the production of TNFα in BV2 cells, a microglial cell line, and bone marrow derived macrophages (BMDM). BV2 and BMDM cells can be treated with different proinflammatory stimuli, including IFNγ, IFNγ, LPS or MDP (muramy1 dipeptide), and the production of TNFα is a marker for inflammatory responses. IFNγ, IFNγ+zVAD and LPS+zVAD can stimulate TNFα production in BV2 cells in a RIPK1 kinase dependent manner, and compounds disclosed herein can attenuate the response in these stimuli.

Necroptosis can occur when RIPK1 is activated by blocking the caspase-8-mediated cleavage of RIPK1. Certain disclosed compounds can inhibit necroptosis by inhibiting the activation of RIPK1. This inhibition leads to blocking the activation of RIPK1 and caspases in RDA. These compounds increase the M1 ubiquitination of RIPK1 in complex I, which is known to be involved in regulating its activation. Thus, disclosed compounds can suppress the activation of RIPK1 by modulating its ubiquitination.

Disclosed compounds block both extrinsic and specific intrinsic apoptosis, attenuate TNF release under different stimuli and induce autophagy. These mechanisms of action can be demonstrated in a neurodegenerative disease model in vivo. The involvement of Death Receptor and the activation of caspase-3 are known to play important roles in ischemic brain injury induced cell death. Also, TNF release is involved in ischemic brain injury. One model is a mouse model of stroke induced by middle cerebral artery occlusion (MCAO). Disclosed compounds have shown reduction in the infarct volume in the brains of the mice and inhibited apoptosis during ischemic brain injury, which reduced inflammation.

Central to the action of the disclosed compounds is that the different mechanisms of action involve TRADD. Disclosed compounds promote the recruitment of TRADD into complex I in TNFα stimulated cells and blocking the activation of RIPK1. TRADD is also involved in protection of RDA and the induction of autophagy by compounds disclosed herein.

The common pathological features of neurodegenerative diseases include neuronal cell death, neuroinflammation and the accumulation of misfolded proteins, such as plaques and tangles in Alzheimer's disease, Lewy bodies in Parkinson's disease, poly-Q aggregation in Huntington's disease and TDP-43 aggregation in ALS. These pathological features are believed to be involved in the onset and progression of these neurodegenerative diseases. Thus, an effective therapeutic strategy for the treatment of neurodegenerative diseases should include the ability to promote the degradation of misfolded neurotoxic proteins as well as to block neuroinflammation and cell death. Apoptosis is involved in mediating neuronal cell death in neurodegenerative diseases. Autophagy is an important cellular degradative and recycling mechanism. Autophagy deficiency leads to the accumulation of protein inclusion bodies and progressive neural deficit. Furthermore, the natural decline of autophagy in human aged brains may contribute to the onset of neurodegenerative diseases by reducing protein turnover and promoting the accumulation of misfolded proteins. Compounds disclosed herein can block apoptosis and induce autophagy to counteract the detrimental pathways leading to neurodegenerative diseases and neural inflammation.

Disclosed herein are methods of reducing apoptosis and promoting autophagy (e.g., blocking apoptosis and/or inducing autophagy) in a subject in need thereof, comprising administering to the subject a compound of Formula I or II or a compound selected from

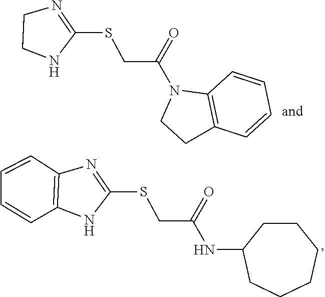 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same. In some embodiments, the method is a method of blocking apoptosis and/or inducing autophagy in a subject in need thereof, or the method is an in vitro method of blocking apoptosis and/or inducing autophagy in a cell; and the blocking apoptosis and/or inducing autophagy occurs in the presence of adapter protein TRADD. Disclosed herein are methods of promoting cellular recruitment of TRADD to complex I in a cell comprising contacting the cell with a compound of Formula I or II or a compound selected from and

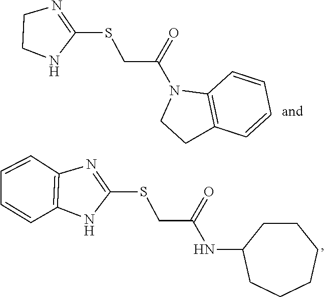 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same.

Disclosed herein are methods of treating neurodegenerative disease, ischemic brain injury, amyloidosis, inflammatory bowel diseases, liver diseases or a metabolic disease in a subject in need thereof, comprising administering to the subject a compound of Formula I or II or a compound selected from

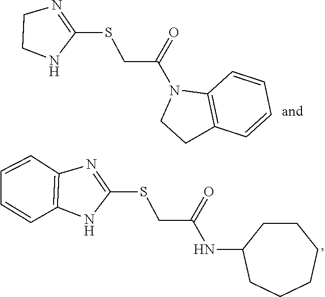 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same. In some embodiments, the method is a method of treating a neurodegenerative disease, and the neurodegenerative disease is selected from Alzheimer's disease, Parkinson's disease, Huntington's disease, frontotemporal lobar degeneration and amyotrophic lateral sclerosis.

Disclosed herein are methods of treating spinal and bulbar muscular atrophy, Huntington's disease, dentatorubral pallidoluysian atrophy, and six spinocerebellar ataxias (SCA 1, 2, 3, 6, 7, and 17), comprising administering to a subject a compound of Formula I or II or a compound selected from

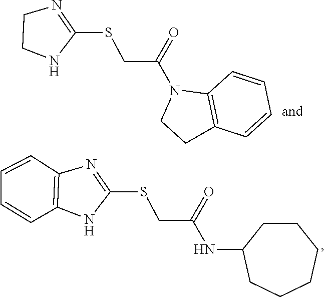 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same.

Disclosed herein are methods of treating Parkinson's disease, Huntington's disease, Alzheimer's disease, amyotrophic lateral sclerosis, hereditary spastic paraplegias, Lafora disease, β-propeller protein-associated neurodegeneration, static encephalopathy of childhood with neurodegeneration in adulthood, Vici syndrome, tauopathy, frontotemporal lobar degeneration, prion disease, and spinocerebellar ataxia type 3, comprising administering to a subject a compound of Formula I or II or a compound selected from

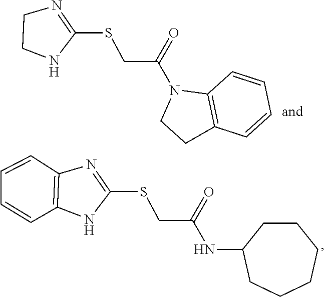 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same.

Disclosed herein are methods of treating Alzheimer's disease, amyotrophic lateral Sclerosis, cerebral ischemia, Creutzfeldt-Jakob disease, Fahr disease, Huntington's disease and related polyglutamine expansion diseases, Lewy body disease, Menke's disease, multiple sclerosis, stroke, and Wilson's disease, comprising administering to a subject a compound of

Formula I or II or a compound selected from and

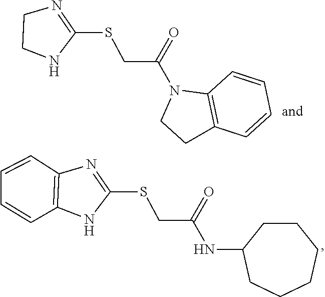 [SMILES: O=C(CSC1=NCCN1)N1CCc2ccccc21 ; O=C(CSc1nc2ccccc2[nH]1)NC1CCCCCC1]

or a pharmaceutically acceptable salt of any of the foregoing, or a pharmaceutical composition comprising the same.

Disclosed herein are methods of treating Alzheimer's disease, Huntington's disease, Parkinson's disease, amyotrophic lateral sclerosis, HIV-associated dementia, cerebral ischemia, amyotrophic lateral Sclerosis, multiple sclerosis, Lewy body disease, Menke's disease, Wilson's disease, Creutzfeldt-Jakob disease, Fahr disease, and progressive supranuclear palsy.

Disclosed herein are methods of treating a disease caused by misfolded protein aggregates. In certain embodiments, the disease caused by misfolded protein aggregates is selected from: Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease.

Disclosed herein are methods of treating cancer e.g., any cancer wherein the induction of autophagy would inhibit cell growth and division, reduce mutagenesis, remove mitochondria and other organelles damaged by reactive oxygen species or kill developing tumor cells.

Disclosed herein are methods of treating a neurodegenerative disease selected from: Adrenal Leukodystrophy, alcoholism, Alexander's disease, Alper's disease, Alzheimer's disease, Amyotrophic lateral sclerosis, ataxia telangiectasia, Batten disease, bovine spongiform encephalopathy, Canavan disease, cerebral palsy, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease, multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis, toxic encephalopathy and combinations of these diseases. In some embodiments, the proteinopathy is al-antitrypsin deficiency, sporadic inclusion body myositis, limb girdle muscular dystrophy type 2B and Miyoshi myopathy Alzheimer's disease, Parkinson's disease, Lewy Body Dementia, ALS, Huntington's disease, spinocerebellar ataxias, spinobulbar muscular atrophy and combinations of these diseases.

Disclosed herein are methods of treating a disease or disorder associated with RIPK1 kinase activity-mediated inflammation. The subject may have neuroinflammatory disorder, such as multiple sclerosis. Multiple sclerosis is a disease in which the body's immune system attacks the central nervous system, which is made up of the brain, spinal cord, and optic nerves. This abnormal response of the immune system damages nerve fibers and the myelin sheath. In aspects of the invention, a compound, composition, or method disclosed herein may be utilized to prevent and/or treat a disease involving neuroinflammation (i.e., a neuroinflammatory disease). These neuroinflammation-related disorders include, but are not limited to, Alzheimer's disease (AD), amyotrophic lateral sclerosis, autoimmune disorders, priori diseases, stroke and traumatic brain injury. Neuroinflammation may be brought about by glial cell (e.g., astrocytes and microglia) activation, which normally serves a beneficial role as-part of an organism's homeostatic response to injury or developmental change. However, dysregulation of this process through chronic or excessive activation of glia contributes to the disease process through the increased production of proinflammatory cytokines and chemokines, oxidative stress-related enzymes, acute phase proteins, and various components of the complement cascades. Examples of diseases that can be treated and/or prevented using the compounds, agents, compositions and methods disclosed herein include Alzheimer's disease and related disorders, presenile and senile forms; amyloid angiopathy; mild cognitive impairment; Alzheimer's disease-related dementia (e.g., vascular dementia or Alzheimer dementia); AIDS related dementia, tauopathies (e.g., argyrophilic grain dementia, corticobasal degeneration, dementia pugilistica, diffuse neurofibrillary tangles with calcification, frontotemporal dementia with parkinsonism, prion-related disease, Hallervorden-Spatz disease, myotonic dystrophy, Niemann-Pick disease type C, non-Guamanian motor neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, tangle only dementia, alpha-synucleinopathy (e.g., dementia with Lewy bodies, multiple system atrophy with glial cytoplasmic inclusions), multiple system atrophies, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function- of chaperones and/or triple A proteins); Huntington's Disease, spinocerebellar ataxia, Freidrich's Ataxia; cerebrovascular diseases including stroke, hypoxia, ischemia, infarction, intracerebral hemorrhage; traumatic brain injury; Down's syndrome; head trauma with post-traumatic accumulation of amyloid beta peptide; familial British dementia; familial Danish dementia; presenile dementia with spastic ataxia; cerebral. amyloid angiopathy, British type; presenile dementia with spastic ataxia cerebral amyloid angiopathy, Danish type; familial encephalopathy with neuroserpin inclusion bodies (fenib); amyloid polyneuropathy (e.g., senile amyloid polyneuropathy or systemic amyloidosis); inclusion body myositis due to amyloid beta peptide; familial and Finnish type amyloidosis; systemic amyloidosis associated with multiple myeloma; Familial Mediterranean Fever; multiple sclerosis, optic neuritis; Guillain-Barre syndrome; chronic inflammatory demyelinating polyneuropathy; chronic infections and inflammations; acute disseminated encephalomyelitis (ADEM); autoimmune inner ear disease (AIED); diabetes; myocardial ischemia and other cardiovascular disorders; pancreatitis; gout; inflammatory bowel disease; ulcerative colitis, Crohn's disease, rheumatoid arthritis, osteoarthritis; arteriosclerosis, inflammatory aortic aneurysm; asthma; adult respiratory distress syndrome; restenosis; ischemia/reperfusion injury; glomerulonephritis; sacoidosis cancer; restenosis; rheumatic fever; systemic lupus erythematosus; Reiter's syndrome; psoriatic arthritis; ankylosing spondylitis; coxarthritis; pelvic inflammatory disease; osteomyelitis; adhesive capsulitis; oligoarthritis; periarthritis; polyarthritis; psoriasis; Still's disease; synovitis; inflammatory dermatosis; and wound healing.

Disclosed herein are methods of treating liver diseases that are characterized by the accumulation of pathological proteins and lipids, and inflammatory bowel diseases, such as Crohn's disease.

In some embodiments, the methods disclosed herein comprise administering a compound selected from

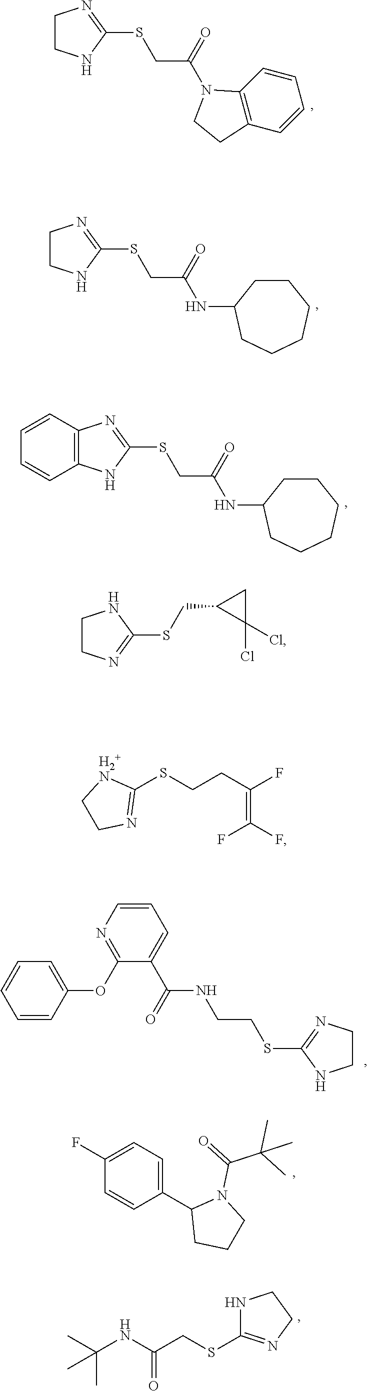

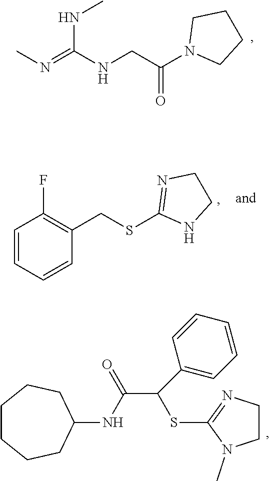 [SMILES: C/N=C(\NC)NCC(=O)N1CCCC1 ; Fc1ccccc1CSC1=NCCN1 ; CN1CCN=C1SC(C(=O)NC1CCCCCC1)c1ccccc1]

or a pharmaceutically acceptable salt of any of the foregoing. In some such embodiments, the compound is

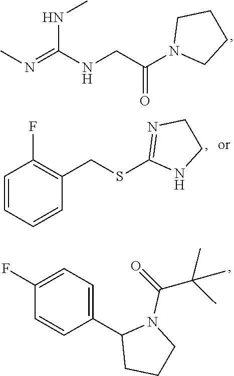

or a pharmaceutically acceptable salt thereof.

Disclosed herein is:

i) a method of treating a neurodegenerative disease, ischemic brain injury, amyloidosis, inflammatory bowel diseases, liver diseases or a metabolic disease in a subject in need thereof, comprising administering to the subject an effective amount of a compound of

Formula I, or

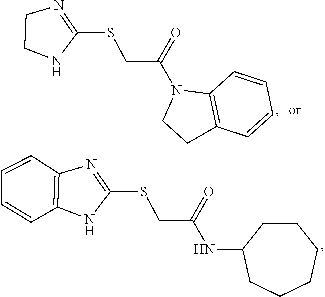 [SMILES: O=C(CSC1=NCCN1)N1CCc2ccccc21 ; O=C(CSc1nc2ccccc2[nH]1)NC1CCCCCC1]

or a pharmaceutically acceptable salt thereof, or
ii) a method of blocking apoptosis and/or inducing autophagy in a subject in need thereof, comprising administering to the subject an effective amount of a compound of Formula I, or

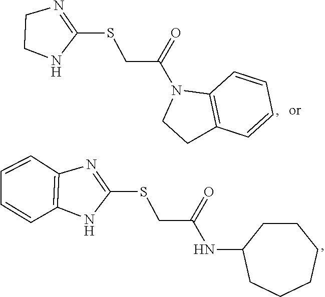 [SMILES: O=C(CSC1=NCCN1)N1CCc2ccccc21 ; O=C(CSc1nc2ccccc2[nH]1)NC1CCCCCC1]

or a pharmaceutically acceptable salt thereof; or
iii) an in vitro method of blocking apoptosis and/or inducing autophagy in a cell, comprising contacting the cell with a compound of Formula I, or

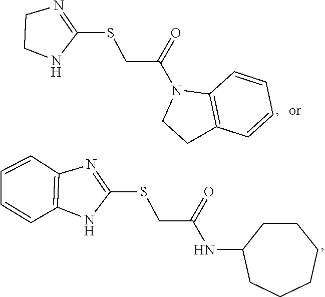 [SMILES: O=C(CSC1=NCCN1)N1CCc2ccccc21 ; O=C(CSc1nc2ccccc2[nH]1)NC1CCCCCC1]

or a pharmaceutically acceptable salt thereof, or
iv) a method of promoting cellular recruitment of TRADD to complex I in a cell, comprising contacting the cell with a compound of Formula I, or

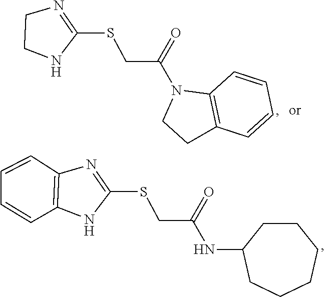 [SMILES: O=C(CSC1=NCCN1)N1CCc2ccccc21 ; O=C(CSc1nc2ccccc2[nH]1)NC1CCCCCC1]

or a pharmaceutically acceptable salt thereof.

Also disclosed herein is a method of treating a neurodegenerative disease, ischemic brain injury, amyloidosis, inflammatory bowel diseases, liver diseases or a metabolic disease in a subject in need thereof, comprising inhibiting TRADD. In certain embodiments, inhibiting TRADD comprises administering a TRADD inhibitor (e.g., a compound disclosed herein).

Also disclosed herein is a method of inhibiting TRADD in a subject in need thereof, comprising administering to the subject a compound disclosed herein (e.g., a therapeutically effective amount of a compound disclosed herein), or a pharmaceutically acceptable salt thereof.

### Exemplification

The invention now being generally described, it will be more readily understood by reference to the following examples, which are included merely for purposes of illustration of certain aspects and embodiments of the present invention, and are not intended to limit the invention.

### SYNTHETIC EXAMPLES

### Example 1. Compound 30 (also referred to as Apt-1 and D18 herein)

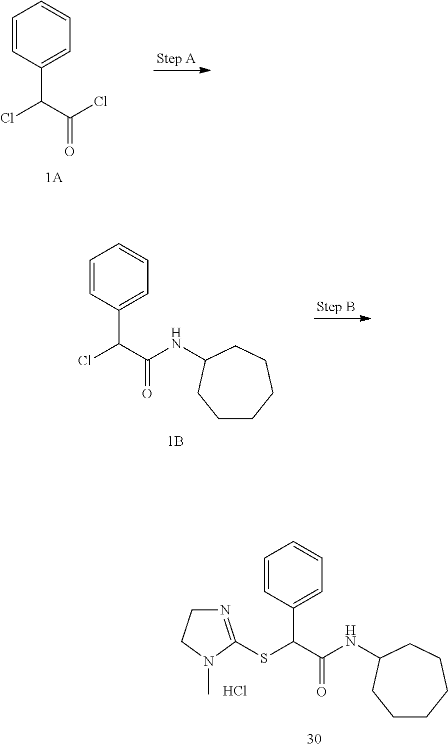 [SMILES: O=C(Cl)C(Cl)c1ccccc1 ; O=C(NC1CCCCCC1)C(Cl)c1ccccc1 ; CN1CCN=C1SC(C(=O)NC1CCCCCC1)c1ccccc1 ; Cl]

Step A: Cycloheptylamine (11.3 g, 0.1 mol) and triethylamine (12 g, 0.12 mol, 1.2 eq) were dissolved in acetonitrile (300 mL) and cooled to −20° C. A solution of 1A (19 g, 0.1 mol) in acetonitrile (30 mL) was added dropwise and then the reaction mixture was allowed to warm to room temperature and stir for 16 hours. The mixture was evaporated, 0.01 M HCl aqueous solution (300 mL) was added and the obtained precipitate was filtered. The material was recrystallized from isopropanol-hexane to give 1B (20 g). Yield: 75%.
Step B: Compound 1B (10 g, 0.037 mol) and 1-methyl-4,5-dihydro-1H-imidazole-2-thiol (3, 4.3 g, 0.037 mol) were dissolved into DMA (200 mL) and the reaction mixture was stirred for 16 hours at 90-100° C. The mixture was allowed to cool to room temperature, diluted with Et₂O (150 mL) and stirred for another hour. The obtained precipitate was filtered to give crude product, which was purified by column chromatography on silica gel to give 30 (6 g). Yield: 47%. The material was further purified by liquid chromatography to give 30 hydrochloride salt.
¹H NMR: (CDCl₃, 400 MHz): δ 9.7 (bs, 1H), 7.4-7.2 (m, 5H), 5.0 (s, 1H), 4.6-4.5 (m, 1H), 3.8-3.6 (m, 2H), 3.4-3.2 (m, 2H), 2.75 (s, 3H), 2.3-2.1 (m, 2H), 1.8-1.4 (m, 10H). LRMS: m/z=346.2

### Example 2. Compounds 29, 33, 35 36, and 42

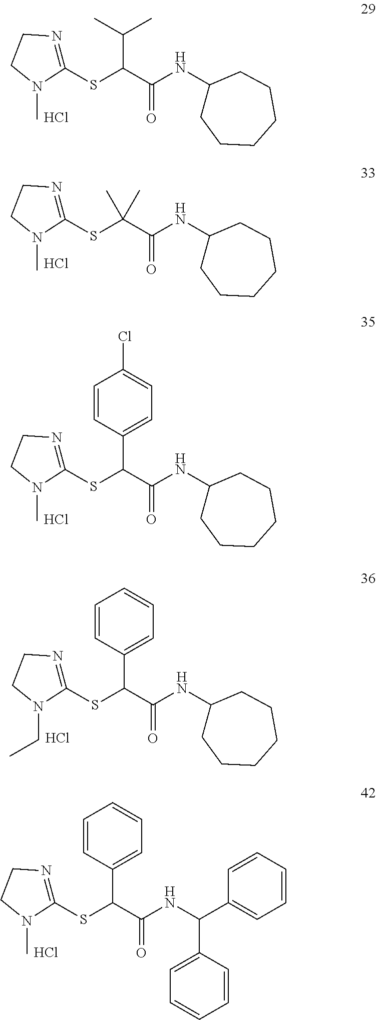

Compounds 29, 33, 35, 36, and 42 were prepared using similar procedures as compound 30. 29: ¹H NMR: (CDCl₃, 400 MHz): δ 9.1 (bs, 1H), 4.55-4.45 (m, 1H), 4.05-4.00 (m, 1H), 3.75-3.65 (m, 2H), 3.35-3.25 (m, 2H), 2.80 (s, 3H), 2.25-2.15 (m, 2H), 1.75-1.40 (m, 10H), 1.05-0.95 (d, 3H), 0.90-0.80 (d, 3H). LRMS: m/z=312.2
33: ¹H NMR: (CDCl₃, 500 MHz): δ 9.2 (bs, 1H), 4.55-4.45 (m, 1H), 3.65-3.55 (m, 2H), 3.35-3.25 (m, 2H), 2.75 (s, 3H), 2.25-2.20 (m, 2H), 1.75-1.45 (m, 16H). LRMS: m/z=298.2 35: LRMS: m/z=380.0
36: ¹H NMR: (d₆-DMSO, 400 MHz): δ 8.95 (bs, 1H), 7.40-7.25 (m, 5H), 5.55 (s, 1H), 4.55 4.45 (m, 1H), 3.55-3.45 (m, 2H), 3.20-3.10 (m, 2H), 3.00-2.96 (m, 2H), 2.20-2.10 (m, 2H), 1.80-1.35 (m, 9H), 1.20 (t, 3H). LRMS: m/z=360.2
42: ¹H NMR: (d₆-DMSO, 400 MHz): δ 10.75 (bs, 1H), 9.78 (m 1H), 7.55-7.10 (m, 15H), 6.50 (s, 1H), 6.10 (m, 1H), 3.85-3.75 (m, 2H), 3.00-2.96 (m, 2H), 2.50 (s, 3H). LRMS: m/z=416.2

### Biological Assays

### 1. Structural and Activity Study for Protection Against Apoptosis Induced by Proteasomal Stress and Activation of Autophagy

Murine RGC5(661W) cells were seeded in 96-well plates (4000 cells per well). Different concentrations of the compounds were added one hour prior to TNFα treatment, followed by the addition of 0.5 ng/ml TNFα plus 0.5 μM 5Z-7-Oxozeaenol to induce RIPK1-dependent apoptosis (RDA). After incubation for 21 hours, cell viability was measured by CellTiter-Glo Luminescent Cell Viability Assay. The data shown in Table 3 indicates that 7-membered carbon rings are significantly effective.

### 2. Efficacy of Compounds on Human Jurkat Cells Prior to Velcade Treatment

Human Jurkat cells were seeded in 96-well plates (20000 cells per well). Different concentrations of the compounds were added one hour prior to Velcade treatment, followed by the addition of 50 nM Velcade. After incubation for 24 hours, cell viability was measured by CellTiter-Glo Luminescent Cell Viability Assay.

### 3. Autophagy Assay

H4-LC3-GFP cells were treated with compounds of different concentrations for 4-24 hrs. The levels of autophagy were determined using LC3-GFP intensity. Autophagy index was determined using this formula (Zhang et al., 2007): Autophagy index=(LC3-GFP dot intensity in sample/Vehicle-1)/(LC3-GFP dot intensity in Rapamycin/Vehicle-1)×100.

### 4. Animals

WT (Catalog No. 004781) and transgenic tauP301S (Catalog No. 008169) mice were from The Jackson Laboratory. All animals were maintained in a pathogen-free environment, and the animal experiments were conducted according to the protocols approved by the Harvard Medical School Institutional Animal Care and Use Committee (IACUC).

### 5. A Multiplexed Chemical Screen

The primary screen was conducted in Jurkat cells treated with the proteasomal inhibitor Velcade to induce apoptosis by proteasomal stress. Inhibition of apoptosis by pan-caspase inhibitor zVAD.fmk was able to partially rescue cell survival, and thus was used as a positive control. ˜170,000 compounds were screened to identify hits which could inhibit apoptosis induced by proteasomal stress better than that of zVAD.fmk. These positive hits were counter-screened against apoptosis induced by 5-fluorouracil (5-FU) to remove generic inhibitors of DNA damage-induced apoptosis. Hits that protected against apoptosis induced by Velcade, but not 5-FU, were further evaluated for their ability to induce autophagy using H4-GFP-LC3 cells. Finally, the hits were further tested in RIPK1 dependent apoptosis (RDA) assay of extrinsic apoptosis in RGC5 mouse retinal ganglion cells treated with TNFα and 5z-7-Oxozeaenol (5z7, an inhibitor of TAK1).

### 6. Stereotaxic Surgery and Apt-1 Delivery

Intracranial injections of synthetic tau-preformed fibrils (pffs) into PS19 mice accelerate the transmission of pathological tau tangle-like aggregates throughout the brain and thus, provide a model for AD pathology and tauopathy. Eight-week-old PS19 mice (P301S tau), expressing T34 isoform of human P301S mutant tau under the control of the mouse prion promoter, were deeply anesthetized with isoflurane and immobilized in a stereotaxic frame. The stereotaxic injections were made using predetermined coordinates with a Hamilton syringe under aseptic conditions. All injected animals were observed during and after surgery, and an analgesic was administered after surgery. T40/PS recombinant tau pffs (2 μg/l) were injected into both sides of hippocampus of PS19 mice (ML, ±1.8 mm; AP, −2.2 mm; DV, 1.8 mm). The total volume injected 2.5 l/injection for all mice. The mice were then either dosed immediately or waited for three weeks before delivering Apt-1 intracerebroventricularly via an ALZET micro-osmotic pump (ALZET Micro-Osmotic Model 1002). ALZET brain infusion kit was used for delivery into lateral ventricles (ML, −1.0 mm; AP, −0.5 mm; DV, 2.0 mm) at a rate of 0.25 l/h. The ALZET micro-osmotic pumps were fixed on the skulls of the mice by instant adhesive and the skin incision was closed by suture. Apt-1 (20 mM) in the ALZET micro-osmotic pumps was renewed every two days and the Apt-1 delivery was maintained for a month in the immediate dosing groups or one week in the delayed dosing group. Apt-1 treatment resulted no apparent difference in survival or behavior of the mice. At the end of Apt-1 dosing for one month or one week, the mice were sacrificed and perfused by PBS and the hippocampi from half of the brains were dissected and analyzed by immunoblotting after lysis in RIPA buffer (50 mM Tris-HCl pH 7.5, 150 mM NaCl, 1% NP-40, 0.1% SDS). The other half of the brains were fixed in 4% paraformaldehyde and embedded in paraffin blocks from which 5-μm-thick sections were processed for immunohistochemistry (IHC) using AT8 (specific for pathological tau phosphorylated at Ser202/Thr205, 1:10,000; Invitrogen), MC1 (specific for a pathological conformation of tau, 1:2000) and TUNEL (terminal deoxynucleotidyl transferase dUTP nick end labeling).

### 7. Pharmacokinetic (DMPK) Study of Apt-1

WT mice (2 months old) were deeply anesthetized with isoflurane and immobilized in a stereotaxic frame using predetermined coordinates under aseptic conditions. All animals were observed during and after surgery, and an analgesic was administered after surgery. Apt-1 (20 mM, 100 μl, release rate: 0.25 μl/h) was delivered intracerebroventricularly via an ALZET micro-osmotic pump (ALZET Model 1002) and ALZET brain infusion kits into lateral ventricles (ML, −1.0 mm; AP, −0.5 mm; DV, 2.0 mm). The ALZET micro-osmotic pumps were fixed on the skulls of the mice by instant adhesive and the skin incision was closed by suture. At 1 h, 6 h and 24 h after the onset of delivery, cerebrospinal fluid (CSF) was carefully withdrawn by a Hamilton syringe from lateral ventricles (ML, −1.0 mm; AP, −0.5 mm; DV, 2.0 mm) for DMPK analysis. The mice were sacrificed at 24 h and the hippocampi were dissected for DMPK analysis. The number of mice is 3 for each time point. The concentrations of Apt-1 in collected samples were measured by HPLC as a custom service by the Scripps Research Institute Florida.

### 8. Organotypic Brain Slice Preparation

PS19 mice (4 months old) were anesthetized with isoflurane prior to decapitation. The brain was removed and immediately immersed in ice-cold cutting solution (2.5 mM KCl, 5 mM MgCl₂, 11 mM D-Glucose, 238 mM Sucrose, 26 mM NaHCO₃, 1 mM NaH₂PO₄, 1 mM CaCl₂)). The cerebellum was trimmed off and the caudal end of the brain was glued onto the cutting table of the vibratome (LEICA VT1000 S, Germany). The brain was cut in coronal slices of 350 mm with an amplitude of 1.5 mm, a frequency of 75 Hz and a velocity of 0.1 mm/s. The slices were collected and stored in ice-cold cutting solution before floating onto semi-porous membrane inserts (Millipore, Millicell-CMLow Height Culture Plate Inserts, Schwalbach, Germany). Slices were cultured at 37° C. and 5% CO₂ in a culture medium consisting of 394 ml MEM, 10% normal horse serum, 5 mg/mL penicillin, 5 mg/mL streptomycin, 2.5 ml L-glutamine, 1 mM MgSO₄, 11 mM D-Glucose, 238 mM Sucrose, 5 mM NaHCO₃, 1 mM CaCl₂), 26.6 mM HEPES, 0.024 ml 25% ascorbic acid and 0.5 mg Insulin. Medium was changed every other day. Slices are maintained for 14 days in vitro prior to treatment.

### 9. Construction and Transfection of Plasmids

Full-length cDNAs for mouse/human TRADD were PCR-amplified from the plasmid library and cloned into pcDNA3.1 using Phanta Max Super-Fidelity DNA Polymerase (Vazyme Biotech Co., Ltd) with appropriate tags. Mutant hTRADD were generated using MutExpress II mutagenesis kit (Vazyme Biotech Co., Ltd). For protein purification, cDNA encoding truncated hTRADD (aal-179, WT or mutant) were cloned into pET-28a plasmid for E. coli expression using ClonExpress II One Step Cloning Kit (Vazyme Biotech Co., Ltd), cDNA encoding GST-tagged hTRADD (Full length or aa180-312) was cloned into EcoRV/NotI sites in pEBG plasmid for mammalian expression, cDNAs encoding mVenus- and Flag-tagged TRADD-N(aal-179) and mCerulean- and Flag-tagged TRAF2-C(aa310-501) were cloned into pLenti plasmid for mammalian expression. All plasmids were verified by DNA sequencing and the details of the plasmid sequences are available upon request. Transient transfections of H4 and SH-SY5Y cells were performed using Lipofectamine 3000 (Invitrogen) according to the manufacturers' instructions. Briefly, cells were plated at a density of 5×10⁴ cells per well in a 12-well plate and transfected with a total of 1 μg DNA per well for 24 h. Medium was changed the day after transfection.

### 10. Generation of Knockdown, Knockout and Reconstitution Lines

Cells were stably infected with shRNA against mouse Traf2 (TAGTTCGGCCTTTCCAGATAA), human BECNI 3′-UTR (CTCTGTGTTAGAGATATGA) or scramble control in the pLKO.1 lentiviral background. For CRISPR/Cas9 system-mediated gene knockout, guide RNA against human Tradd: sgTradd-1 (GCGCGCAGCTCCAGTTGCAG), sgTradd-2 (GCGCCCCCTCGCGGTAGGCG); Atg5: sgAtg5-1 (GCTTCAATTGCATCCTTAGA), sgAtg5-2 (GTGCTTCGAGATGTGTGGTT) in the Lenti-CRISPR v2 lentiviral background. Viral supernatant fractions were collected 48 h after the transfection. Cleared supernatant fraction was filtered through a 0.45-mm filter. Polybrene (8 mg/ml) was supplemented to viral supernatant fractions. 24 h after infection, cells stably expressing shRNA or sgRNA were obtained by selection with 5 μg/ml puromycin. BECNI 3′-UTR shRNA expressing H4 cells were infected with lentiviral particles expressing Flag-Beclin 1 (WT or mutant). Polyclonal populations were screened until WT and mutant lines were generated that had near endogenous Beclin 1 reconstitution levels.

### 11. Analysis of Cytotoxicity and Viability

The rates of cell death were measured in triplicate or quadruplicate in a 96-well or 384-well plate by using SYTOX Green Nucleic Acid Stain (Invitrogen) or ToxiLight Non-destructive Cytotoxicity BioAssay Kit (Lonza). The intensity of luminescence was determined in an EnSpire Multimode Plate Reader (PerkinElmer). Cytotoxicity is expressed as percentages of cell death per well after deducting the background signal in non-induced cells and compared to that of the maximal cell death with 100% Lysis Reagent. The rates of cell viability were determined by using CellTiter-Glo Luminescent Cell Viability Assay (Promega) following the manufacturer's protocol and the results are expressed as percentages of luminescence intensity per well after deducting the background signal in blank well and compared to that of the viability in the non-treated wells. Concentration of drugs used for inducing or inhibiting cell death, mTNFα: 1 ng/ml; 5Z-7-Oxozeaenol: 0.5 μM; Velcade: 50 nM; Apt-1/ICCB-19/ICCB-19i/Nec-1s: 10 μM.

### 12. Caspase-8 Activity Assay

Caspase-Glo 8 assay (Promega) was used to detect the activity of caspase-8 in cells and in vitro by following manufacture's protocol. Briefly, 2×10⁵ cells (MEFs) were plated in 6-well plates and treated as indicated in 2 ml for the indicated times. After treatment, media was removed, and 300 l 0.5% NP-40 lysis buffer (50 mM Tris-HCl pH 7.5, 150 mM NaCl and 0.5% NP-40) was added to each well, cells were scraped and lysates were left on ice for 5 min. 10 l of lysate per condition were transferred into a 384-well plate and 90 l of Caspase-Glo 8 reagent supplemented with MG-132 (30 μM) was added to each well. Plates were wrapped in foil and gently mixed using a plate shaker at 300-500 rpm for 30 sec. Reactions was allowed to proceed by incubation at room temperature for 1 h. Caspase-8 activity was read on a luminometer.

### 13. Complex-I/II Purification

Cells were seeded in 15 cm dishes and treated as indicated with Flag-TNFα (50 μg/ml). To terminate treatment, media was removed and plates were washed with 50 mL of ice cold PBS. Plates were frozen at −80C until all time points were acquired. Plates were thawed on ice and cells were lysed in 0.5% NP-40 lysis buffer (50 mM Tris-HCl pH 7.5, 150 mM NaCl and 0.5% NP-40) supplemented with protease inhibitors and N-Ethylmaleimide (2.5 mg/ml). Cell lysates were rotated at 4° C. for 30 min then clarified at 4° C. at 14,000 rpm for 30 min. Proteins were immunoprecipitated from cleared protein lysates with 20 μl of anti-Flag M2 beads (Sigma) with rotation overnight at 4° C. 4×washes in 0.5% NP-40 buffer with N-Ethylmaleimide were performed, and samples eluted by boiling in 50 μl 1×SDS loading buffer. For complex-II purification cells were seeded in 10 cm dishes and treated as indicated using media containing TNFα (10 ng/ml) and zVAD (20 μM). Cells were lysed on ice in 0.5% NP-40 lysis buffer. Cell lysates were rotated at 4C for 30 min then clarified at 4C at 14,000 rpm for 10 min. 20 μl of protein G Sepharose (Sigma), after pre-blocking for 1 h with lysis buffer containing 1% BSA, were incubated with FADD antibody (1.5 mg antibody/mg protein lysate) and the mixture was incubated in rotation with cleared protein lysates 4 h at 4C. The samples were then washed four times in lysis buffer and eluted by boiling in 50 l 1×SDS loading buffer. Concentration of compounds used in complex-III purification: 10 μM (Apt-1 or ICCB-19).

### 14. Long-Lived Protein Degradation Assay

H4 cells were cultured with L-[3,4,5-³H(N)]-leucine (0.1 μCi/ml) (PerkinElmer Life Sciences) for 24 h and chased in media with nonradioactive leucine for 18 h to let the degradation of short-lived proteins happen. Then the media was changed and incubated for additional 6 h along with different compounds (10 μM Apt-1, 10 μM ICCB-19, 10 μM ICCB-19i, 1 μM rapamycin). The media were recovered and treated with 10% trichloroacetic acid to separate trichloroacetic acid-soluble (amino acids) and trichloroacetic acid-insoluble (proteins) fractions. The cells were completely dissolved with 1N NaOH. Radioactivity was measured with a liquid scintillation analyzer (PerkinElmer). Long-lived protein degradation was calculated by dividing trichloroacetic acid-soluble radioactivity in the media by total radioactivity detected in the cells and media. The values were expressed as changes in fold from the value obtained in control cells.

### 15. NanoBiT Protein-Protein Interaction (PPI) Assay

The Nano-Glo Live Cell assay kit (Promega) was used as follows: HEK293T cells were seeded at 7.5×10³ cells per well in a white, clear-bottom 96-well plate 12 h before transfection (10 ng LgBiT-fused construct and 10 ng SmBiT-fused construct). After 24 h incubation, medium was removed and replaced with 100 μl Opti-MEM medium for 1 h at 37C. The Nano-Glo reagent was prepared as per manufacturer's instructions and added to each well immediately before the luminescence reading was taken. Luminescence was measured at 1 min intervals for 10 min on a plate reader and reported as relative light units (RLU). For quantitative comparison of LgBiT-SmBiT interactions, the peak values at the 2-3 min time point were used. Concentration of compounds used in NanoBiT assay: 10 μM (Apt-1, ICCB-19 or ICCB-19i).

### 16. Protein Expression and Purification

Recombinant WT and mutant His-TRADD-N(aal-179) protein fragment was expressed in BL21 (DE3) E. coli after induction with 0.5 mM IPTG overnight at 16C. ¹⁵N-labeled TRADD-N domain protein was purified from E. co/i grown at 16C in minimal medium. Bacteria were harvested and disrupted by a high-pressure homogenizer and purified by Ni²⁺ affinity resin (GE Healthcare). All proteins were further purified by size exclusion chromatograph on a Superdex 75 column (GE Healthcare) in a buffer containing 20 mM imidazole (pH6.6), 200 mM NaCl, 20 mM DTT and 0.05% NaN₃. All NMR samples were in the same buffer at concentrations between 0.2-0.4 mM with 90% H₂O/10% D₂O. Proteins were exchanged into assay buffer (120 mM NaCl, 20 mM NaH₂PO₄/Na₂HPO₄, pH 7.4) by dialysis for thermal shift assay.

### 17. NMR Spectroscopy

The ¹⁵N-HSQC spectra of ¹⁵N-labeled TRADD-N domain protein were acquired in a buffer containing 20 mM imidazole (pH6.6), 200 mM NaCl, 20 mM DTT and 0.05% NaN₃ at 25C, on a 600 MHz Bruker Avance II spectrometer using a Prodigy cryoprobe. For the spectra of 0.2 mM TRADD-N with 0.5 mM Apt-1 ligand, the data were collected with 8 number of scans for each FID, 512 complex points in the direct ¹H dimension and 128 complex points in the indirect ¹⁵N dimension. For the spectra of 0.4 mM TRADD-N with 0.5 mM ICCB-19, or 0.5 mM ICCB-19i, the data were collected with 2 number of scans for each FID, 512 complex points in the direct ¹H dimension and 128 complex points in the indirect ¹⁵N dimension. The spectra were processed and analyzed using Bruker Topspin software.

The STD method relies on the selective saturation of protein signals that do not overlap with resonances of the ligand. This saturation quickly propagates throughout the protein by spin diffusion and is transferred to the ligand, which only occurs if it is bound, leading to reduced intensities of the ligand. The STD spectra were acquired on a 400 MHz spectrometer (ICCB-19 and Apt-1) or 800 MHz spectrometer (ICCB-19i). The samples for STD NMR were prepared as 13 μM TRADD-N or TRADD-N(G121A) with 1 mM Apt-1, ICCB-19 or ICCB-19i in 0.5 mL of PBS in D₂O (10%). The on-resonance irradiation was performed at a chemical shift of −0.5 ppm, whereas the off-resonance irradiation was conducted at 37 ppm. The spectra were acquired using the following parameters: spectral window of 6.4 kHz, number of scans at 320, acquisition time of 2 s, and repetition time of 3 s. The decrease in signal intensity in STD spectrum, resulting from the transfer of saturation from the protein to the ligand, is evaluated by subtracting the on-resonance spectrum from the off-resonance spectrum.

### 18. Thermostability Shift Assay

To determine stability, purified proteins were made to a final concentration of 1 μg/l. SYPRO Orange dye was added to the protein to make a final concentration of 2×. Compounds were added in the mix with a final concentration of 250 μM or as indicated and incubated at 4C for 1 h. The experiments were performed in 384-well plates specific for real-time PCR instrument with a total volume of 20 μl/well. The assay plate was covered with a sheet of optically clear adhesive to seal each well. The assay plate was centrifuged at 800×g for 2 min at 25C to collect solutions in the bottom of the well and remove bubbles. The assay plate was placed into the Applied Biosystems QuantStudio 6 Real-Time PCR System. The reaction was run from 25C, ramping up in increments of 0.05° C./s to a final temperature of 95C with fluorescence detection throughout the experiment to generate a dataset. Melting temperature of the protein (Tm) was determined by performing non-linear fitting of the dataset to a Boltzmann Sigmoidal curve in GraphPad Prism with the following equation: Y=Bottom+(Top-Bottom)/[1+exp(Tm-X/Slope)], where Y=fluorescence emission in arbitrary units; X=temperature; Bottom=baseline fluorescence at low temperature; Top=maximal fluorescence at top of the dataset; Slope=describes the steepness of the curve, with larger values denoting shallower curves.

### 19. Surface Plasmon Resonance (SPR)

The binding affinity between ICCB-19/Apt-1 and TRADD-N was analyzed at 25C on a BIAcore T200 machine with CM5 chips (GE Healthcare). PBS-P buffer (GE Healthcare) was used for all measurements. For surface plasmon resonance (SPR) measurements, Flag-tagged TRADD-N protein was purified from HEK293T cells by anti-Flag affinity gel and eluted by 3×Flag peptide. The protein was further purified by size exclusion chromatograph on a Superdex 75 column (GE Healthcare) in a buffer containing 20 mM imidazole (pH 6.6), 200 mM NaCl, 20 mM DTT. The protein was dialyzed into PBS and diluted to a final concentration of 40 μg/ml in NaOAc buffer (pH 4.5) before immobilization on CM5 chip. ˜5000 response units of protein were immobilized on the chip with a running buffer composed of PBS-P. Reference was used to normalize the response unit (RU) values of protein. A series of compound concentrations ranging from 0.3125 to 10 μM was tested at 30 l/min flow rate. The contact time is 100 s and dissociation time is 120 s. When the data collection was finished in each cycle, the sensor surface was regenerated with PBS-P buffer. DMSO solvent correction was performed following the BIAcore T200 Guide. Binding curves were displayed, and equilibrium binding constants (K_{D}) for the interaction were determined using the steady-state affinity method incorporated in the BIAEVALUATION 4.1 software (GE Healthcare).

### 20. In Vitro FRET Assay

mVenus- and Flag-tagged TRADD-N(mVenus-TRADDN-Flag) and mCerulean- and Flag-tagged TRAF2C (Flag-TRAF2C-mCerulean) was expressed in HEK293T cells for 48 h. Then cells were lysed in NP-40 buffer followed by immunoprecipitation using anti-Flag affinity gel. The proteins were eluted by 5 mg/ml 3×Flag peptide and exchanged into assay buffer (120 mM NaCl, 20 mM NaH₂PO₄/Na₂HPO₄, pH 7.4) by dialysis for FRET assay. The proteins were added into Coming black 96-well microtiter plates in triplicates at a final concentration of 1 μM. Apt-1 was incubated with the proteins for 1 h before measurement. Measurements were performed on a fluorescent plate reader (Victor3, 1420 Multilabel counter, Perkin Elmer). The following filter set was used: mCerulean filter set (excitation: 430/15 nm, emission: 460/20 nm); mVenus filter set (excitation: 485/15, emission 535/15); FRET filter set (430/15 nm, emission 450-600 nm).

### 21. Mass Spectrometry and Data Analysis

For complex I mass spectrometry analysis, MEFs were treated with Flag-TNFα in the presence or absence of ICCB-19 (10 μM) for indicated time. The binding proteins of TNFR1 in immunoprecipitation pulldown by anti-Flag-beads were trypsin digested. The peptides were analyzed on Q Exactive HF-X Hybrid Quadrupole-Orbitrap Mass Spectrometer (Thermo Scientific). Protein identification and quantification were performed by MaxQuant. The tandem mass spectra were searched against UniProt mouse protein database. The precursor and fragment mass tolerance were set as 20 ppm. The FDR at peptide spectrum match level and protein level was controlled below 1%. The unique peptides plus razor peptides were included for quantification.

For mass spectrometry analysis of ubiquitination sites of Beclin 1, Flag-tagged mBeclin 1 isolated from HEK293T cells expressing this construct was trypsin-digested on beads followed by immunoprecipitation. The resulting peptides were subjected to enrichment of diGly peptides using antibody against ubiquitin remnant motif (K-F-GG) (PTM Biolabs Inc). The enriched diGly peptides were analyzed on the Q Exactive HF-X mass spectrometer (Thermo Scientific). The identification and quantification of diGly peptides was done by MaxQuant. The tandem mass spectra were searched against UniProt mouse protein database together with a set of commonly observed contaminants. The precursor mass tolerance was set as 20 ppm, and the fragment mass tolerance was set as 0.1 Da. The cysteine carbamidomethylation was set as a static modification, and the methionine oxidation as well as lysine with a diGly remnant were set as variable modifications. The FDR at peptide spectrum match level were controlled below 1%.

The effect of Apt-1 on the binding partners of Beclin 1 was characterized by mass spectrometry. The proteins obtained by immunoprecipitation against Flag-tagged Beclin 1 in cells with or without Apt-1 treatment were trypsin digested. The resulting peptides in three replicates were analyzed on a Thermo Scientific Orbitrap Fusion Tribrid mass spectrometer. The protein identification and quantification were done by MaxQuant³⁷. The tandem mass spectra were searched against the UniProt human protein database and a set of commonly observed contaminants. The precursor mass tolerance was set as 20 ppm, and the fragment mass tolerance was set as 0.5 Da. The cysteine carbamidomethylation was set as a static modification, and the methionine oxidation was set as a variable modification. The false discovery rate at the peptide spectrum match level and protein level was controlled to be <1%. The unique peptides plus razor peptides were included for quantification. The summed peptide intensities were used for protein quantification.

### 22. Molecular Modeling and Docking Methods

The 3D atom coordinates of TRADD and TRAF2 interaction complex were obtained from PDB (https://www.rcsb.org) with PDB ID of 1F3V. The TRADD part of this 3D structure served as the protein receptor in the following induced-fit docking procedure performed with the molecular simulation software suite Schrödinger (version 2018-1, Schrödinger, LLC, New York, N.Y., 2018). The receptor was first prepared with the Protein Preparation Wizard. The structure was preprocessed following default settings except no waters were deleted at this step, then hydrogen bond assignment and restrained minimization were performed in the refinement step, followed by removing the water molecules with less than 3 H-bonds to non-waters. The 3D structures of the small molecules were next prepared by LigPrep with no ionization but stereoisomers were generated. The prepared structures of TRADD receptor and small molecules were then submitted for induced-fit docking to predict the binding modes. At the beginning of this step, to define the binding site we inspected the interface of TRADD and TRAF2 interaction and set the docking pocket as the cavity around the center of residues Ile72, Ala122 and Argl46. Considering the surface residue flexibility, we specified refinement of the residues within 9 Å of the ligand during the induced-fit docking process.

### 23. Fluorescence Microscopy

Cells were seeded at 2.5×10⁴ cells per well on poly-L-lysine coated glass cover slips and transfected as described. Cells were fixed in 4% paraformaldehyde, followed by permeabilization with 0.1% Triton X-100. Nuclei were stained using DAPI (Sigma). Cells expressing GFP or RFP-fusion proteins were imaged with an Olympus Fluoview FV1000 confocal microscope (Olympus) using a 40×objective. For GFP-LC3 and DsRed-FYVE puncta quantification, the average spot intensity in 1000 cells from each indicated sample was determined. Images were processed using ImageJ and Photoshop CC. Concentration of compounds used to induce or block autophagy: 10 μM (Apt-1, ICCB-19, ICCB-19i or Spautin-1), for 6 h or as indicated.

### 24. In Vivo Delivery of TNFα

WT mice (n=10, male, 8 weeks of age) were injected intravenously via the tail vein with mTNFα (9.5 μg/mouse) after an intraperitoneal injection of Apt-1 (20 mg/kg) 30 min before. Control mice (n=9) received an equal amount of vehicle 30 min before mTNFα challenge. Kaplan Meier survival curve was determined.
25. Vps34 lipid kinase assay
HEK293T cells were transfected with Flag-Beclin 1 for 18 h and then treated with ICCB-19, ICCB-19i, Apt-1 (10 μM) for another 6 h. Flag-Beclin 1 was immunoprecipitated by anti-Flag to isolate Beclin 1/Vps34 complex. Immunoprecipitated beads were added with sonicated phosphatidylinositol (1 μl of 5 mg) and ATP (1 μl of 10 mM) in 30 μl reaction buffer (40 mM Tris (pH 7.5), 20 mM MgCl₂, 1 mg/ml BSA) for 30 min at room temperature. Wortmannin (10 μM) was used as a control and added into the reaction to inhibit Vps34. The conversion of ATP into ADP levels was measured by an ADP-Glo Kinase Assay Kit (Promega) according to the manufacturer's instructions.

### 26. KINOMEscan Profiling

KINOMEscan profiling was used to assess the interaction of Apt-1 with a panel of 97 kinases as a custom service (DiscoverX/Eurofins, San Diego, Calif. USA). Briefly, DNA-tagged recombinant kinases were produced in E. coli. The assay plates with kinases were incubated at room temperature with shaking for 1 h and the affinity beads were washed with wash buffer (1×PBS, 0.05% Tween 20). The beads were then re-suspended in elution buffer. The kinase concentration in the eluates was measured by qPCR. Apt-1 were screened at 10M, and the results for primary screen binding interactions are reported as % Ctrl, where lower numbers indicate stronger hits in the matrix.

### 27. Quantification and Statistical Analysis

All cell death data are presented as mean±s.d. of one representative experiment. Each experiment was repeated at least 3 times. Mouse data are presented as mean±s.e.m. of the indicated n values. Quantifications of immunoblots were performed by ImageJ, the densitometry data are adjusted to loading control and normalized to control treatment. Error bars for immunoblot analysis represent the standard error of the mean between densitometry data from three unique experiments. Curve fitting and statistical analyses were performed with GraphPad Prism 8.0 software, using either unpaired two-tailed Student's t-test for comparison between two groups, or one-way ANOVA with post hoc Dunnett's tests for comparisons among multiple groups with a single control, or two-way ANOVA with post hoc Bonferroni's tests for comparisons among different groups. Statistical comparisons for series of data collected at different time points were conducted by two-way ANOVA. Significance of in vivo survival data was determined by the log-rank (Mantel-Cox) test. Differences were considered statistically significant if P<0.05(*); P<0.01 (**); P<0.001(***); and n.s., non-significant.

Table 1 below shows the apoptosis IC₅₀ values for Jurkat cells induced by Velcade and the autophagy index.

TABLE 1

| | Apoptosis of | | |
|---|---|---|---|
| | Jurkat cells | | |
| | induced by | Autophagy | |
| | Velcade | index (H4- | |
| Compounds | (IC50 μM) | LC3-GFP) | Structure |
| ICCB-19 | 1.1 | 50.2 | 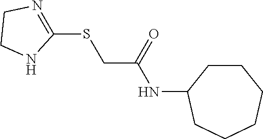 [SMILES: O=C(CSC1=NCCN1)NC1CCCCCC1] |
| ICCB-17 | 13.4 | 18.5 | 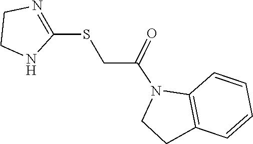 [SMILES: O=C(CSC1=NCCN1)N1CCc2ccccc21] |
| 1 | 6.2 | 40.5 | 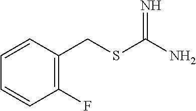 [SMILES: N=C(N)SCc1ccccc1F] |
| 2 | 4.4 | 34.4 | 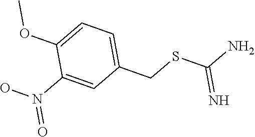 |
| 3 | 32.1 | 23.3 | 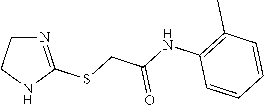 [SMILES: Cc1ccccc1NC(=O)CSC1=NCCN1] |
| 4 | 10.5 | 48.2 | 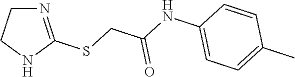 [SMILES: Cc1ccc(NC(=O)CSC2=NCCN2)cc1] |
| 5 | 46.4 | 26.5 | 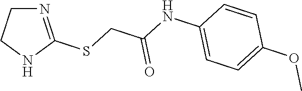 [SMILES: COc1ccc(NC(=O)CSC2=NCCN2)cc1] |
| 6 | ND | 52.2 | 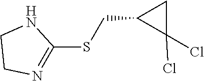 [SMILES: ClC1(Cl)C[C@H]1CSC1=NCCN1] |
| 7 | 6.84 | 43.1 | 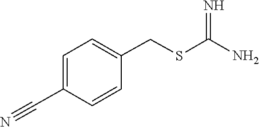 [SMILES: N#Cc1ccc(CSC(=N)N)cc1] |
| 8 | 17.3 | 53.4 | 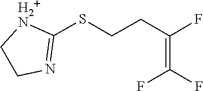 [SMILES: FC(F)=C(F)CCSC1=NCC[NH2+]1] |
| 9 | ND | 47.1 | 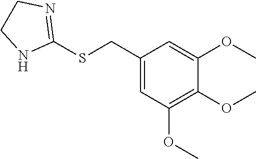 [SMILES: COc1cc(CSC2=NCCN2)cc(OC)c1OC] |
| 10 | ND | 42.0 | 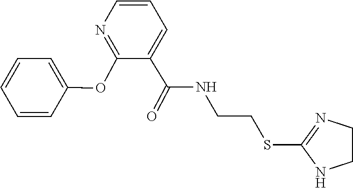 [SMILES: O=C(NCCSC1=NCCN1)c1cccnc1Oc1ccccc1] |
| 11 | ND | 42.4 | 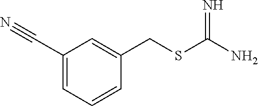 [SMILES: N#Cc1cccc(CSC(=N)N)c1] |

*ND = not disclosed

TABLE 2
Table 2 shows the RIPK1-dependent
apoptosis of Murine RGC5(661W) cells.

| | RGC5 T/5z7 | |
|---|---|---|
| Name | IC50 (μM) | Structure |
| ICCB-19 | 0.18 | 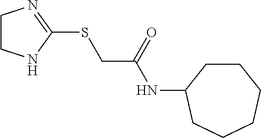 [SMILES: O=C(CSC1=NCCN1)NC1CCCCCC1] |
| 12 | 2.36 | 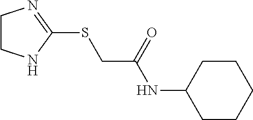 [SMILES: O=C(CSC1=NCCN1)NC1CCCCC1] |
| 13 | 3.42 | 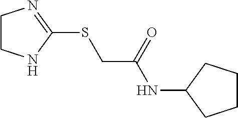 [SMILES: O=C(CSC1=NCCN1)NC1CCCC1] |
| 14 | 4.51 | 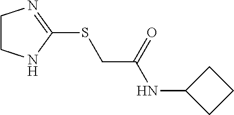 [SMILES: O=C(CSC1=NCCN1)NC1CCC1] |
| 15 | ND | 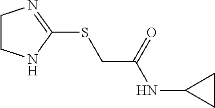 [SMILES: O=C(CSC1=NCCN1)NC1CC1] |

Table 3 shows an SAR study for active derivatives of ICCB-19 in protection against RIPK1-dependent apoptosis and proteasomal stress induced apoptosis.

TABLE 3

| | RGC5 T/5z7 | Jurkat Velcade | |
|---|---|---|---|
| Name | IC50/μM | IC50/μM | Structure |
| ICCB-19 | 2.011 | 1.123 | 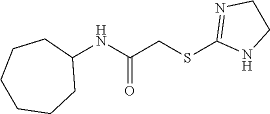 [SMILES: O=C(CSC1=NCCN1)NC1CCCCCC1] |
| 16 | 8.844 | 1.464 | 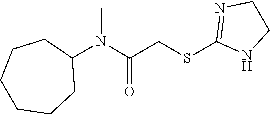 [SMILES: CN(C(=O)CSC1=NCCN1)C1CCCCCC1] |
| 17 | ND | ND | 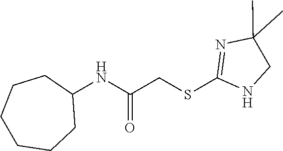 [SMILES: CC1(C)CNC(SCC(=O)NC2CCCCCC2)=N1] |
| 18 | 4.024 | 3.663 | 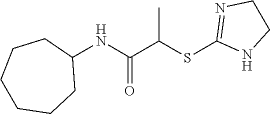 [SMILES: CC(SC1=NCCN1)C(=O)NC1CCCCCC1] |
| 19 | 3.331 | 1.801 | 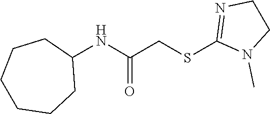 [SMILES: CN1CCN=C1SCC(=O)NC1CCCCCC1] |
| 20 | ND | ND | 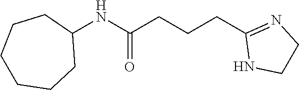 [SMILES: O=C(CCCC1=NCCN1)NC1CCCCCC1] |
| 21 | ND | ND | 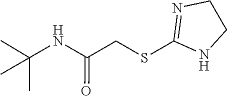 |
| 22 | ND | ND | 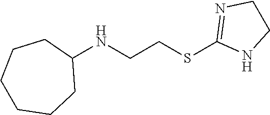 [SMILES: C1CCCC(NCCSC2=NCCN2)CC1] |
| 23 | ND | ND | 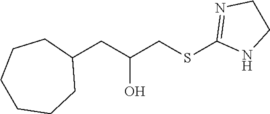 [SMILES: OC(CSC1=NCCN1)CC1CCCCCC1] |
| 24 | ND | ND | 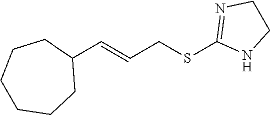 [SMILES: C(=C\C1CCCCCC1)/CSC1=NCCN1] |
| 25 | 2.873 | 1.224 | 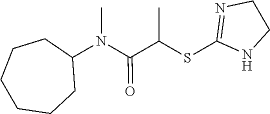 [SMILES: CC(SC1=NCCN1)C(=O)N(C)C1CCCCCC1] |
| 26 | 3.079 | 1.985 | 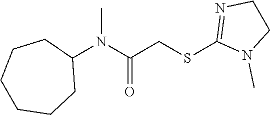 [SMILES: CN1CCN=C1SCC(=O)N(C)C1CCCCCC1] |
| 27 | 2.811 | 3.064 | 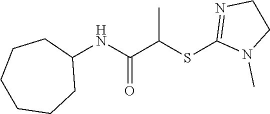 [SMILES: CC(SC1=NCCN1C)C(=O)NC1CCCCCC1] |
| 28 | 2.931 | 0.953 | 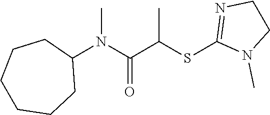 [SMILES: CC(SC1=NCCN1C)C(=O)N(C)C1CCCCCC1] |
| 29 | 1.834 | 3.868 | 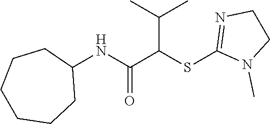 [SMILES: CC(C)C(SC1=NCCN1C)C(=O)NC1CCCCCC1] |
| 30 | 1.280 | 0.799 | 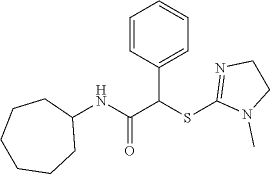 [SMILES: CN1CCN=C1SC(C(=O)NC1CCCCCC1)c1ccccc1] |
| 31 | ND | ND | 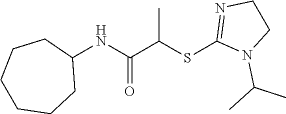 [SMILES: CC(SC1=NCCN1C(C)C)C(=O)NC1CCCCCC1] |
| 32 | ND | ND | 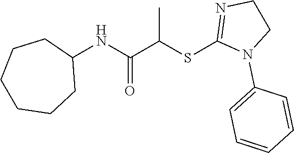 [SMILES: CC(SC1=NCCN1c1ccccc1)C(=O)NC1CCCCCC1] |
| 33 | 1.671 | 1.042 | 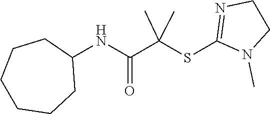 [SMILES: CN1CCN=C1SC(C)(C)C(=O)NC1CCCCCC1] |
| 34 | 2.214 | 0.882 | 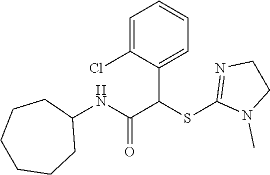 [SMILES: CN1CCN=C1SC(C(=O)NC1CCCCCC1)c1ccccc1Cl] |
| 35 | 1.605 | 2.142 | 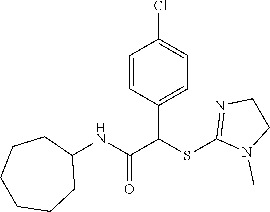 [SMILES: CN1CCN=C1SC(C(=O)NC1CCCCCC1)c1ccc(Cl)cc1] |
| 36 | 1.794 | 1.069 | 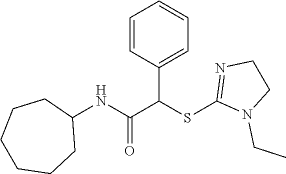 [SMILES: CCN1CCN=C1SC(C(=O)NC1CCCCCC1)c1ccccc1] |
| 37 | 3.046 | 1.167 | 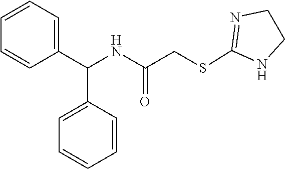 [SMILES: O=C(CSC1=NCCN1)NC(c1ccccc1)c1ccccc1] |
| 38 (also known as ICCB-49) | ND | ND | 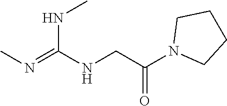 |
| 39 | N.A. | ND | 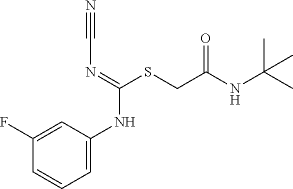 [SMILES: CC(C)(C)NC(=O)CS/C(=N\C#N)Nc1cccc(F)c1] |
| 40 (also known as ICCB-63) | 5.874 | 3.228 | 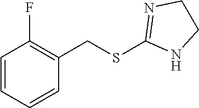 [SMILES: Fc1ccccc1CSC1=NCCN1] |
| 41 | ND | ND | 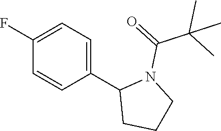 [SMILES: CC(C)(C)C(=O)N1CCCC1c1ccc(F)cc1] |
| 42 | 1.566 | 1.152 | 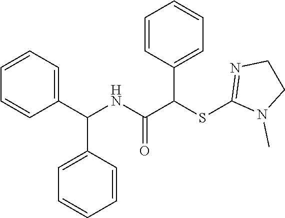 [SMILES: CN1CCN=C1SC(C(=O)NC(c1ccccc1)c1ccccc1)c1ccccc1] |

*ND = not disclosed

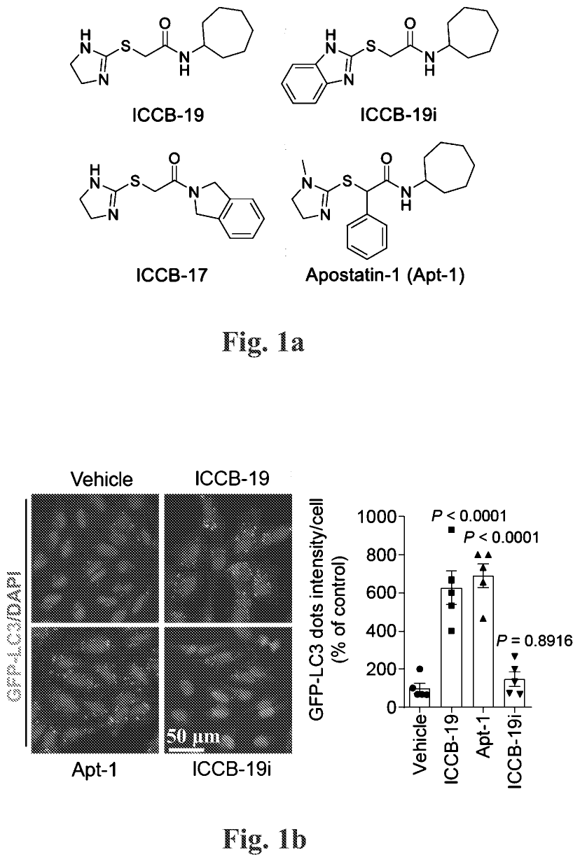

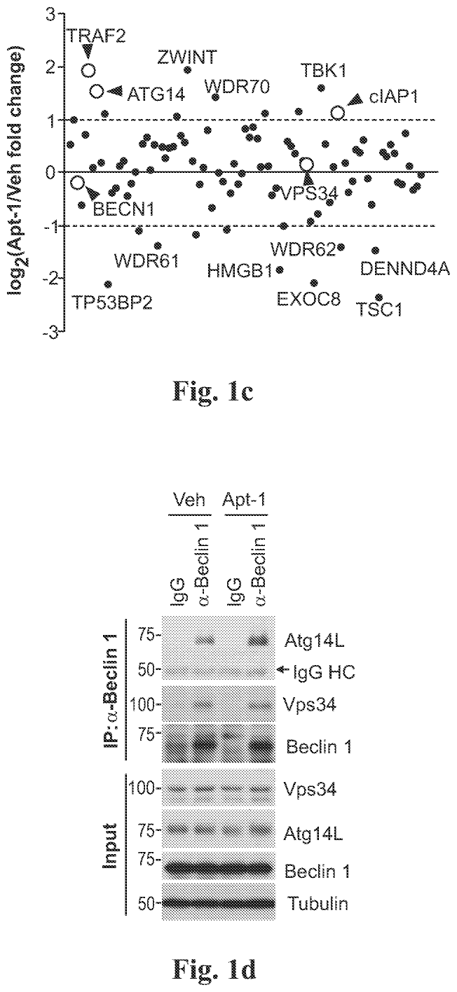

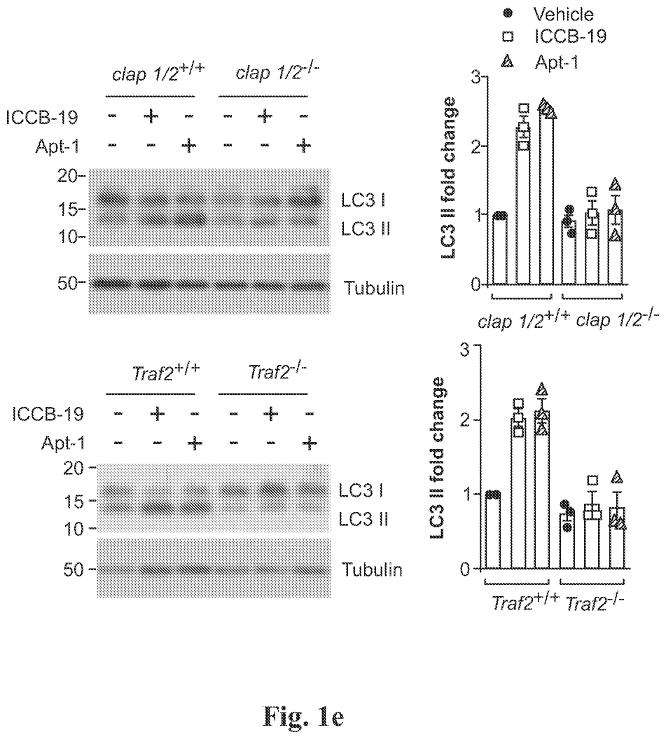

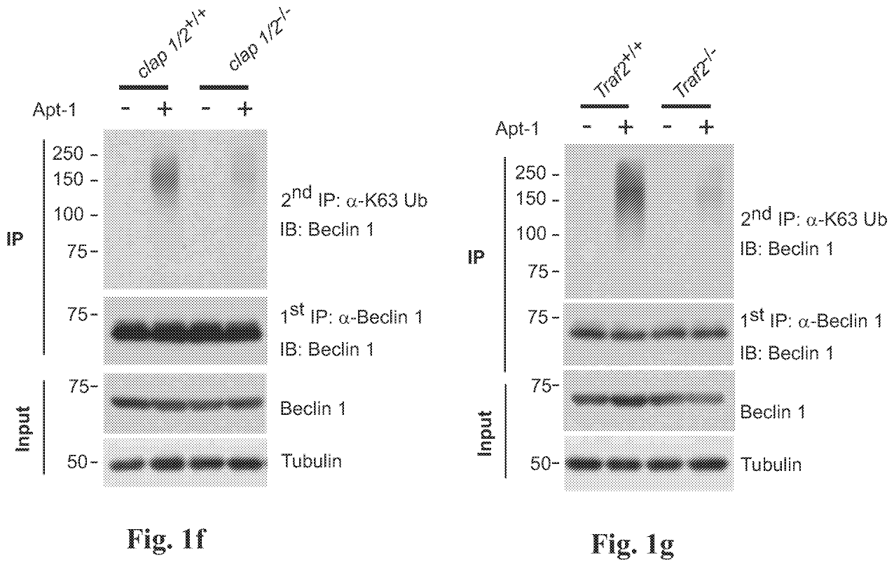

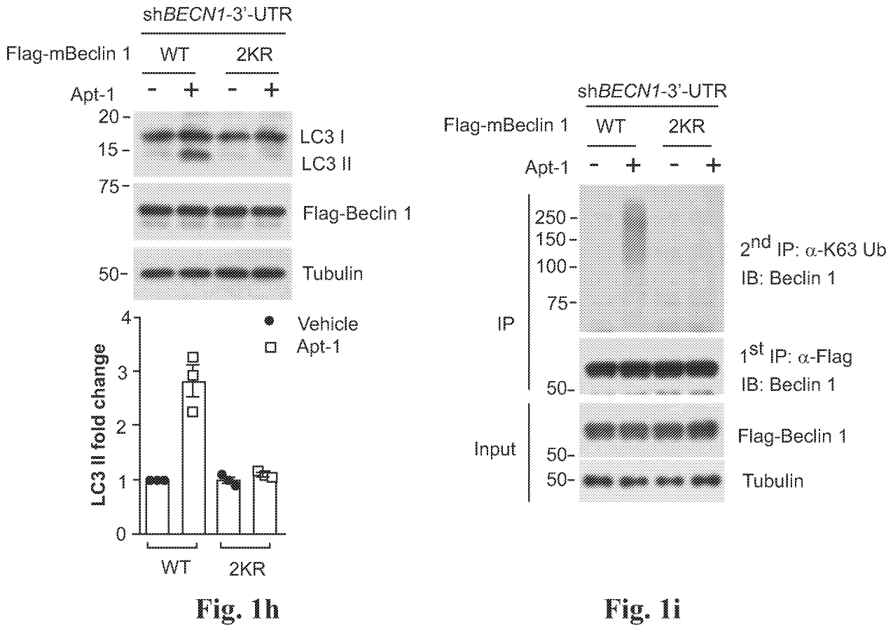

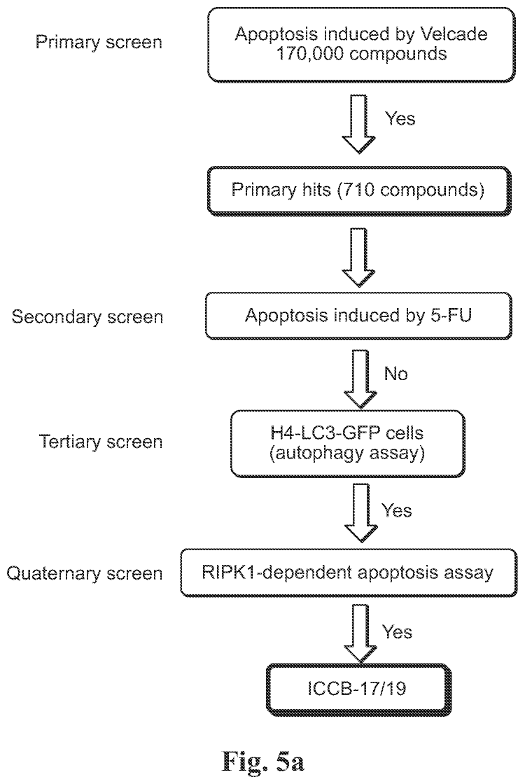

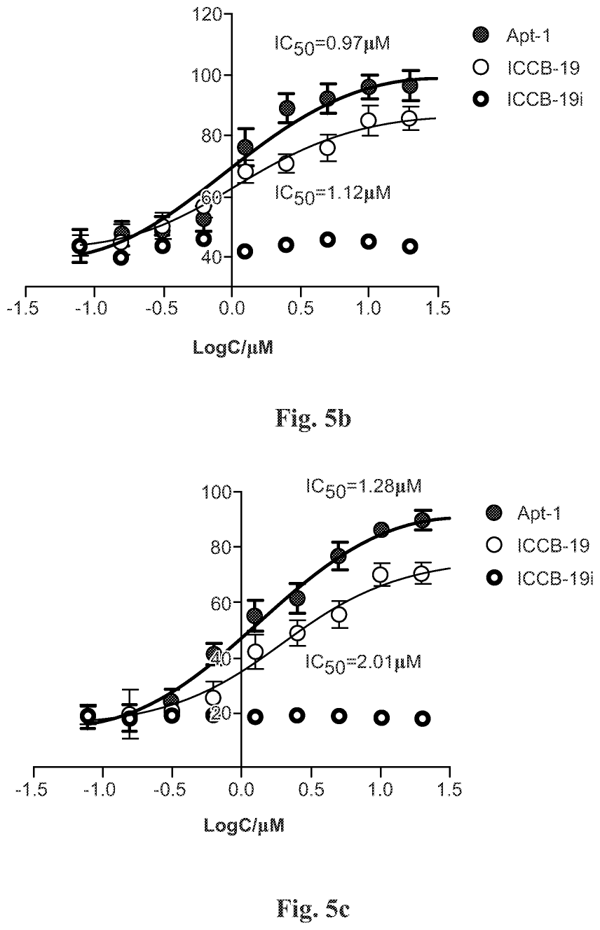

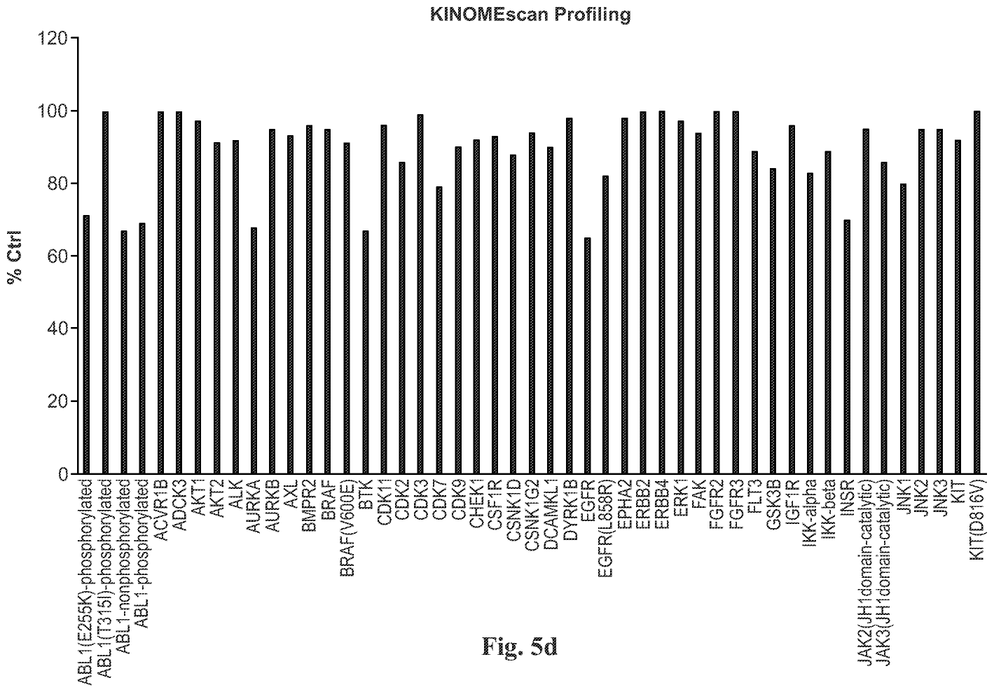

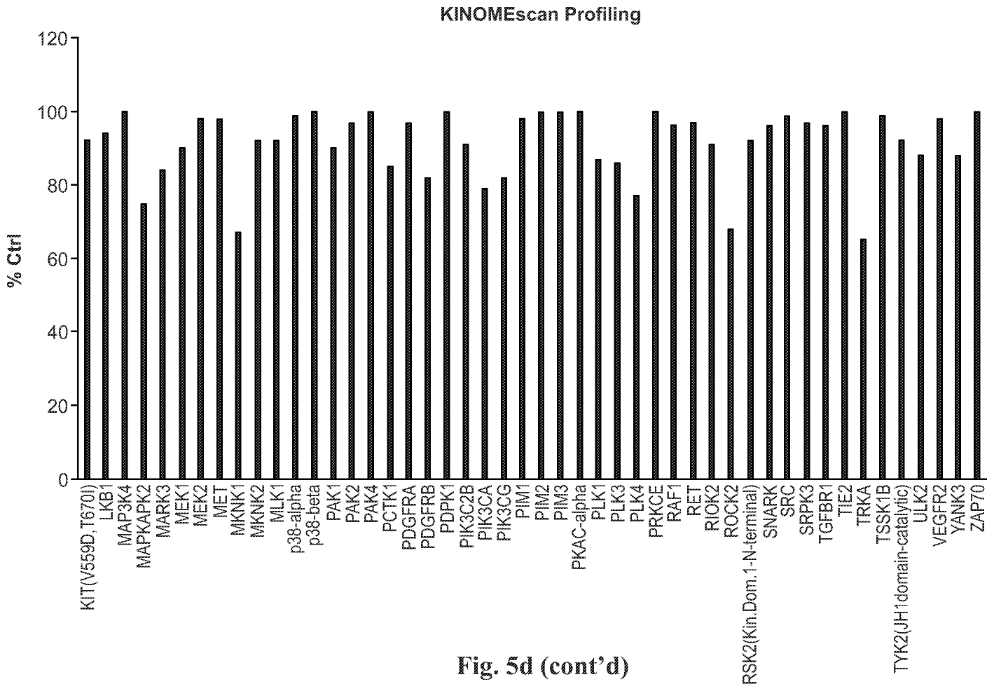

I. Inhibitors of Apoptosis that can Also Activate Autophagy
No targets have been identified which can modulate autophagy to control cellular homeostasis and also inhibit cell death; therefore, we designed and conducted a multiplexed cell-based screen to identify small molecule inhibitors of apoptosis mediated by proteasomal stress and RIPK1-dependent apoptosis (RDA) that are also activators of autophagy (FIG. 5a). This 170,000-compound-library quadruplexed-screen identified two active structural analogs, ICCB-17 and ICCB-19, and a close inactive analog, ICCB-19i (FIG. 1a). An SAR study identified an improved derivative Apostatin-1 (Apt-1). ICCB-19 and Apt-1 inhibited Velcade-induced apoptosis and RDA with IC50 ˜1 μM (FIGS. 5b and 5c). Apt-1 showed no significant off-target effects on 97 kinase targets in KINOMEscan profiling (FIG. 5d).

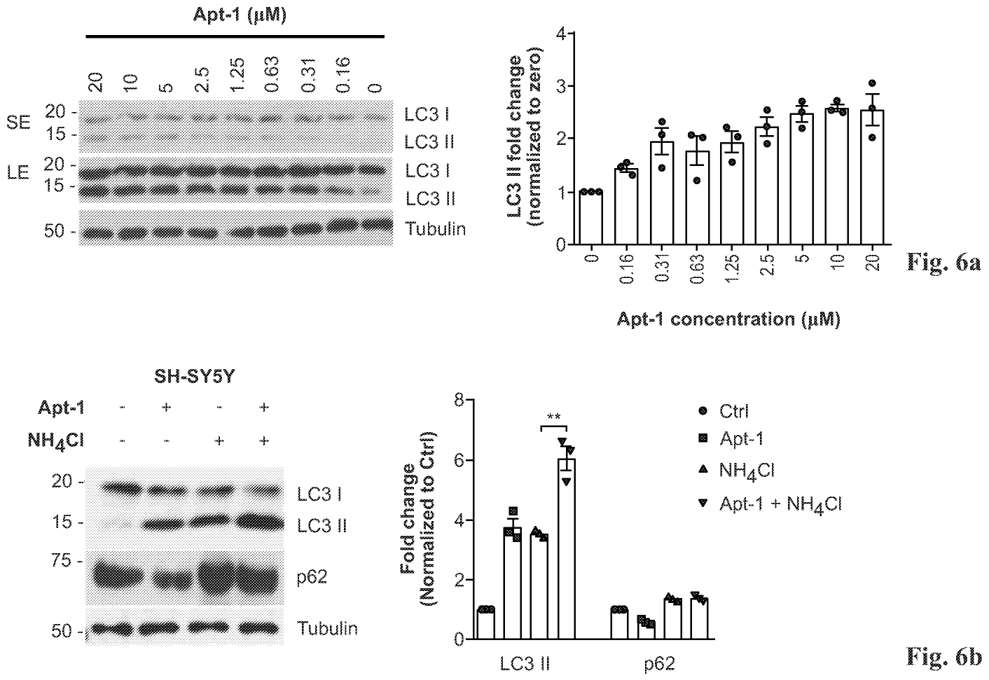

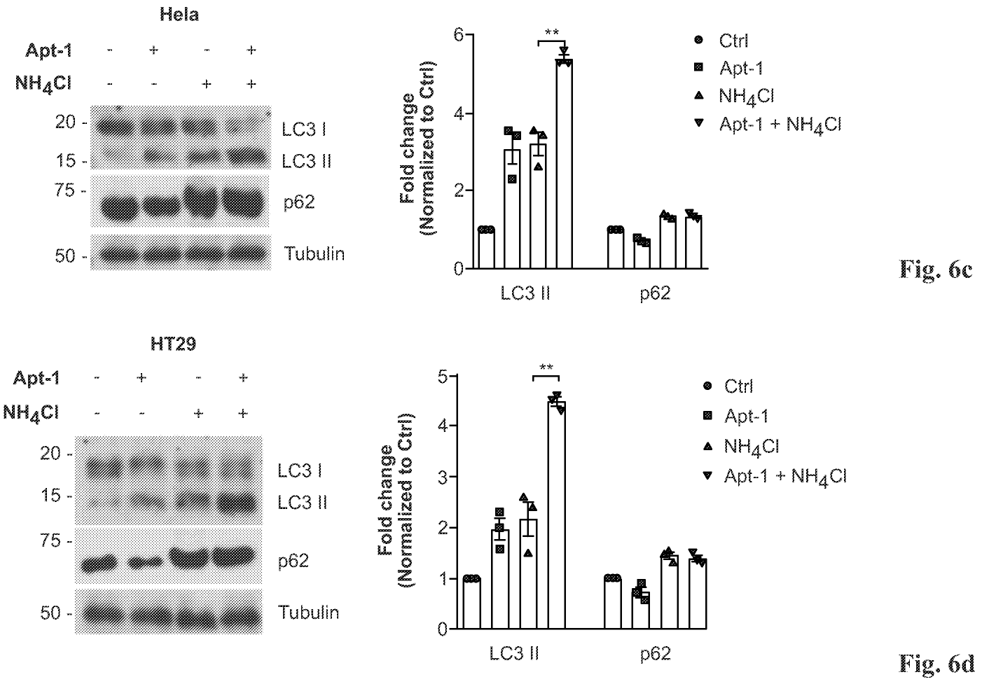

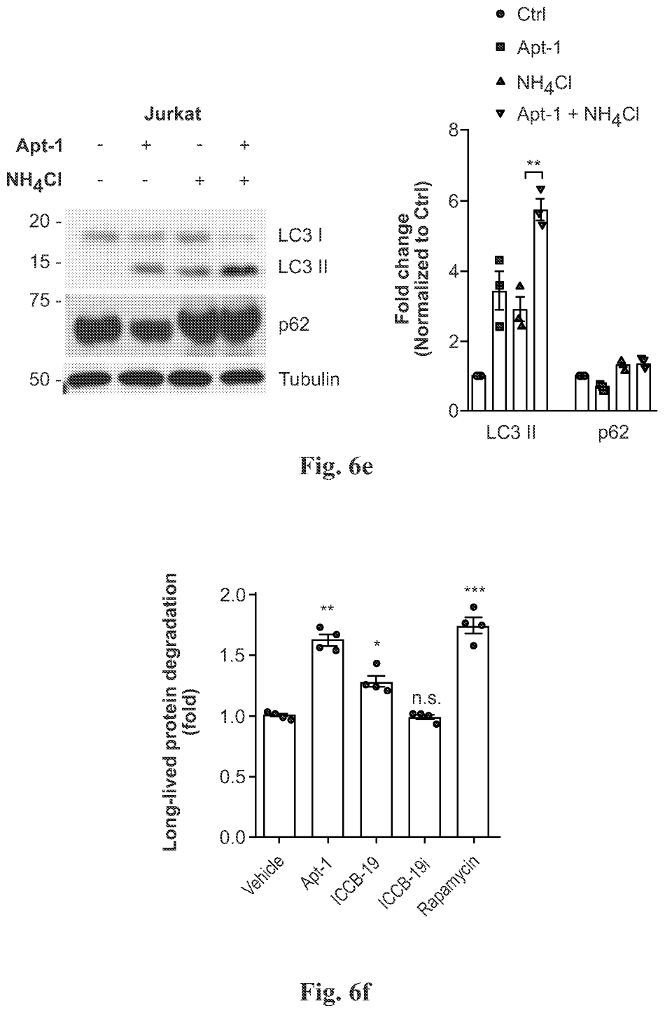

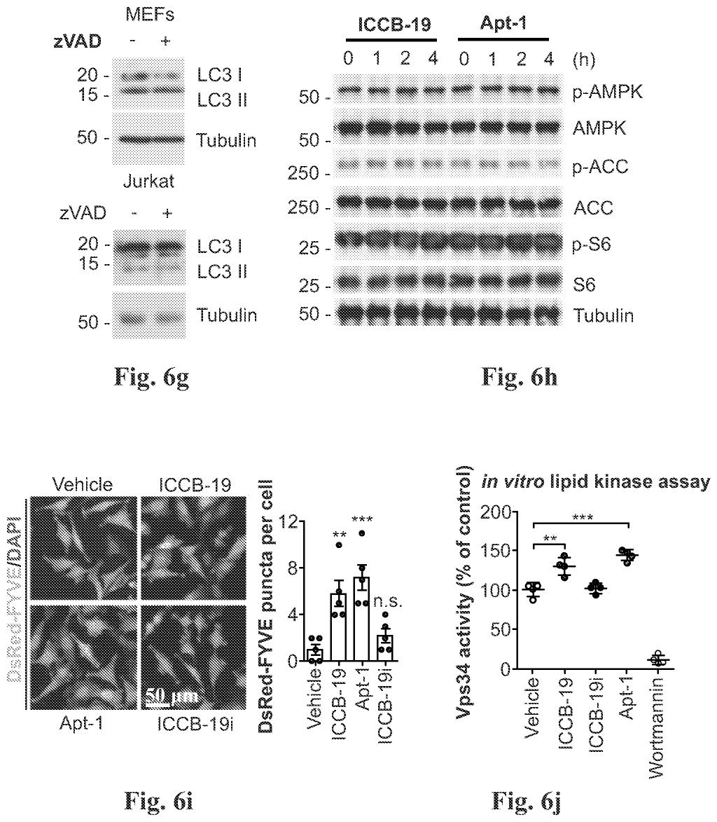

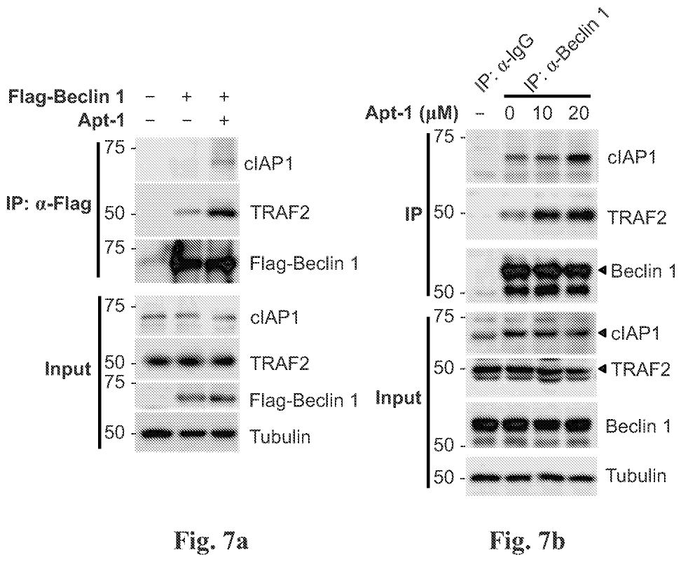

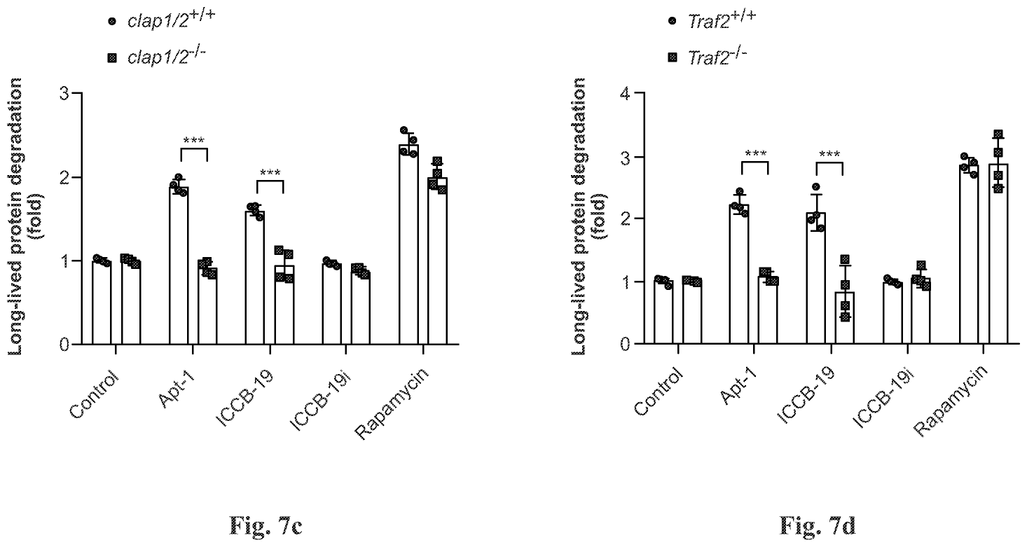

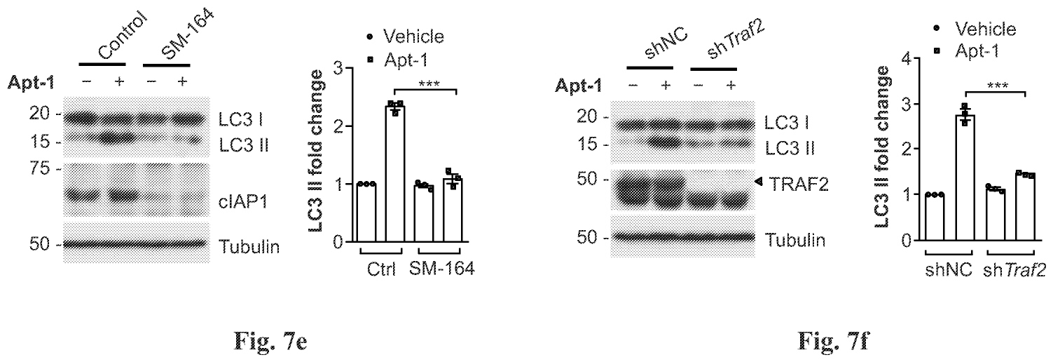

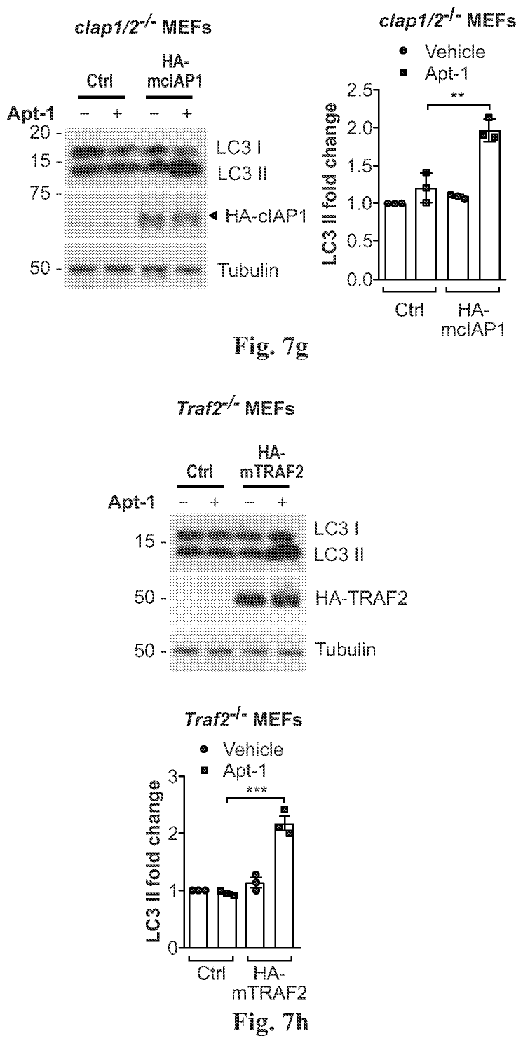

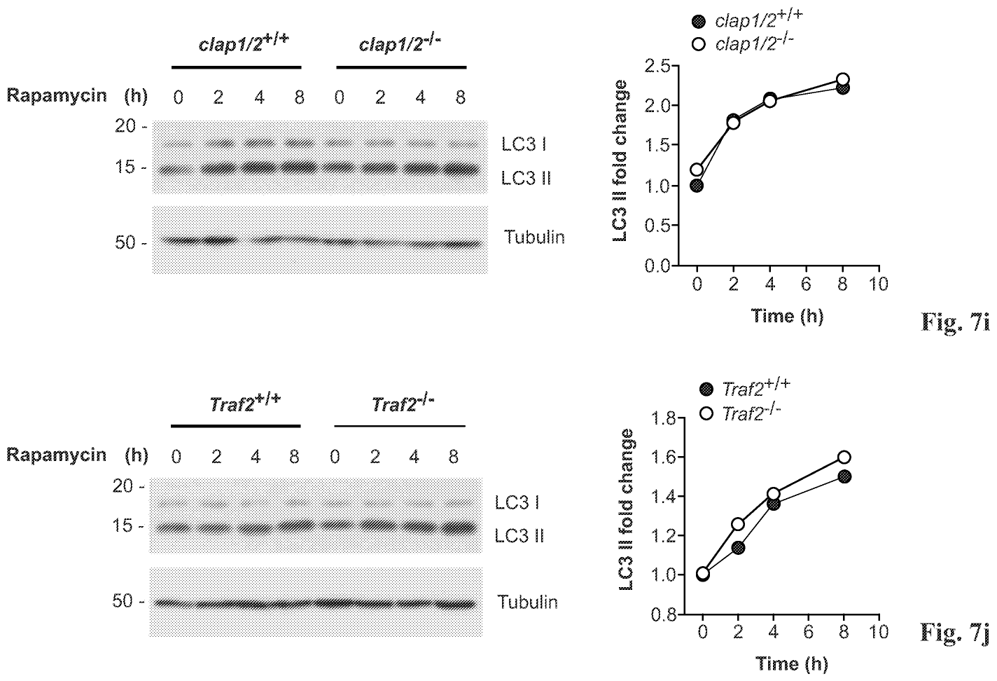

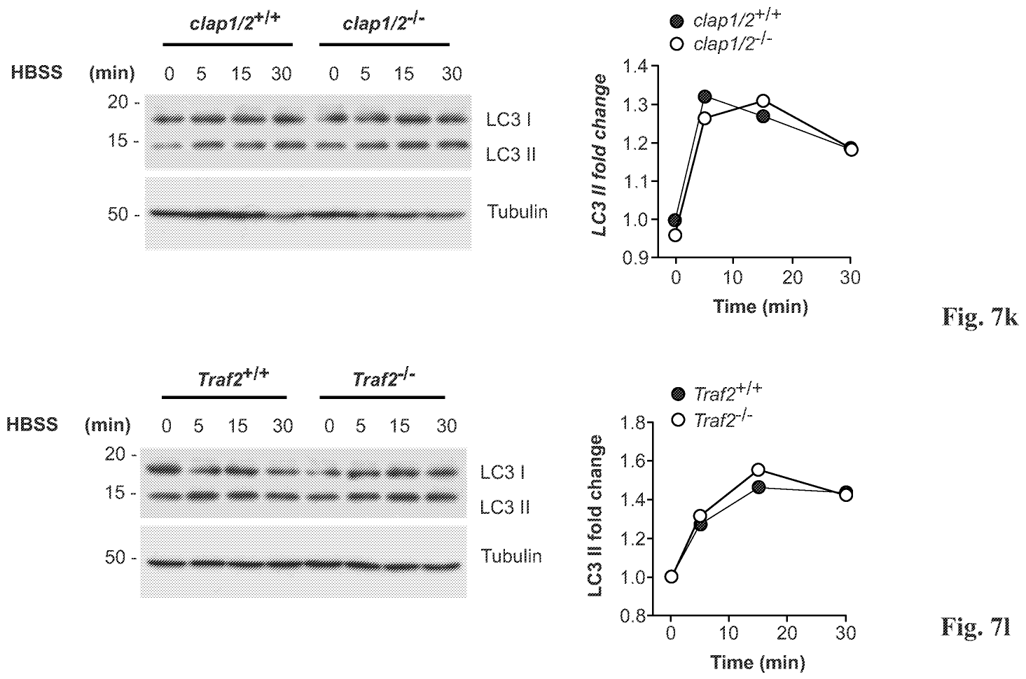

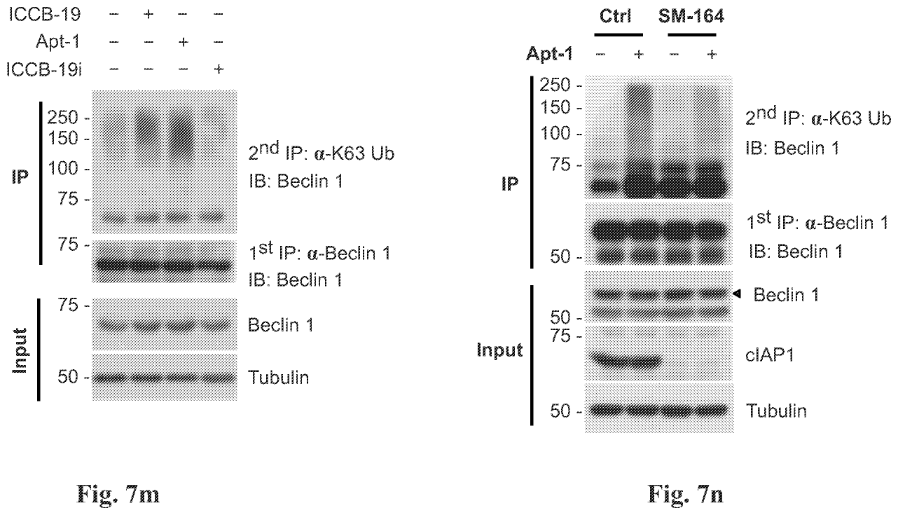

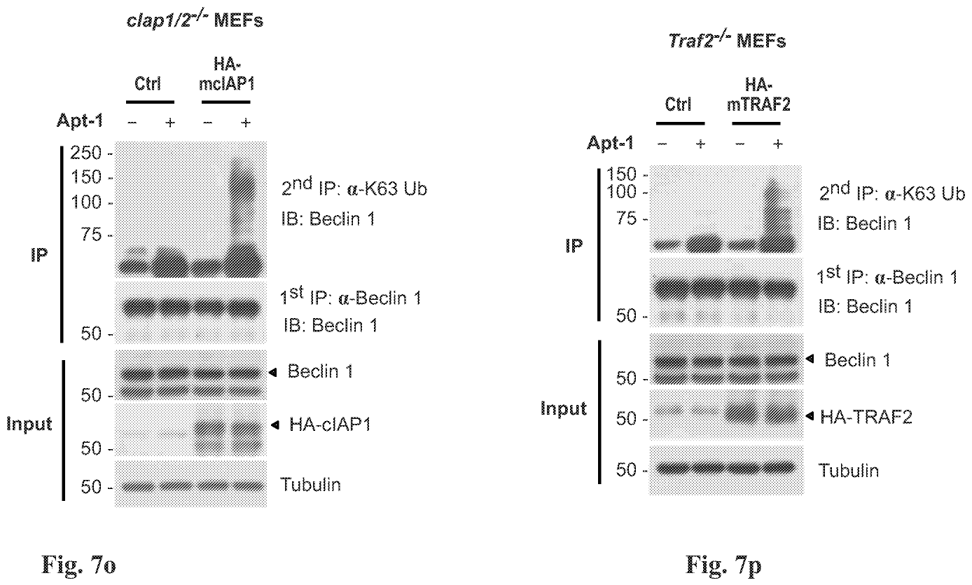

ICCB-19/Apt-1 effectively induced autophagy (FIG. 1b, FIGS. 6a-6e) and degradation of long-lived proteins (FIG. 6f). Caspase inhibitor zVAD.fmk and ICCB-19i had no effect on autophagy (FIG. 6g). ICCB-19 Apt-1 had no effect on mTOR (FIG. 6h). Rather, we found increased levels of DsRed-FYVE dots, an indicator for phosphatidylinositol-3-phosphate (PtdIns3P), a critical autophagy lipid messenger generated by the Atg14L-Beclin 1-Vps34-Vps15 class III PI3 kinase (PI3K-III) complex, following treatment with ICCB-19/Apt-1 (FIG. 6i), suggesting ICCB-19/Apt-1 promote activation of PI3K-III complex. Consistently, treatment with ICCB-19/Apt-1, but not ICCB-19i, increased Vps34 lipid kinase activity (FIG. 6j). Using mass spectrometry and immunoprecipitation-immunoblot, we found that treatment with Apt-1 increased interaction of Beclin 1 with Atg14L, an important activator of Vps34 complex, TRAF2 and cIAP1, but not with Vps34 (FIG. 1c, d; FIG. 7a, 7b). ICCB-19 Apt-1 induced autophagy and long-lived protein degradation was significantly reduced by genetic or pharmacological inhibition of cIAP1/2 or TRAF2, which was rescued by cIAP1 and TRAF2 reconstitution, respectively (FIG. 1e, FIG. 7c-7h). Thus, ICCB-19 Apt-1 induced autophagy involves E3 ubiquitin ligases cIAP1/2 and adaptor TRAF2, which are not required for TORC1 inhibition or starvation-induced autophagy (FIG. 7i-71). Treatment with ICCB-19/Apt-1, but not ICCB-19i, dramatically enhanced Beclin 1 K63 ubiquitination (FIG. 7m). Apt-1-induced K63 ubiquitination of Beclin 1 was reduced by cIAP1/2 or TRAF2 deficiency and restored by reconstitution of cIAP1 or TRAF2, respectively (FIG. 1f, g; FIG. 7n-7p).

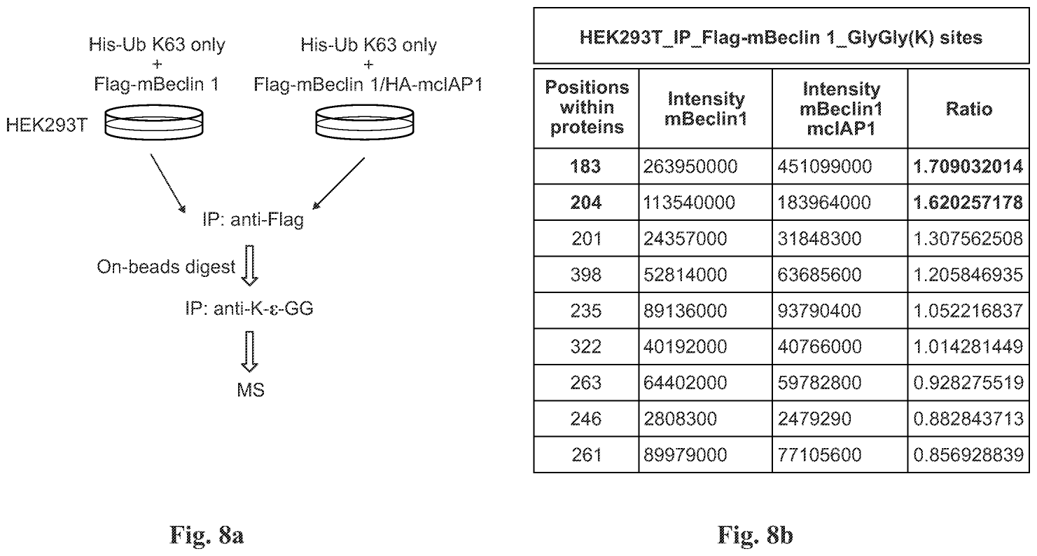

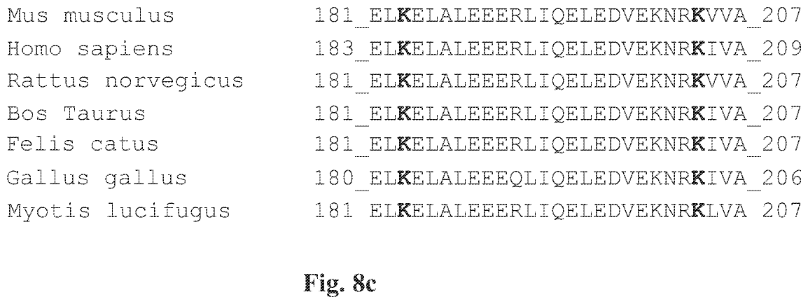

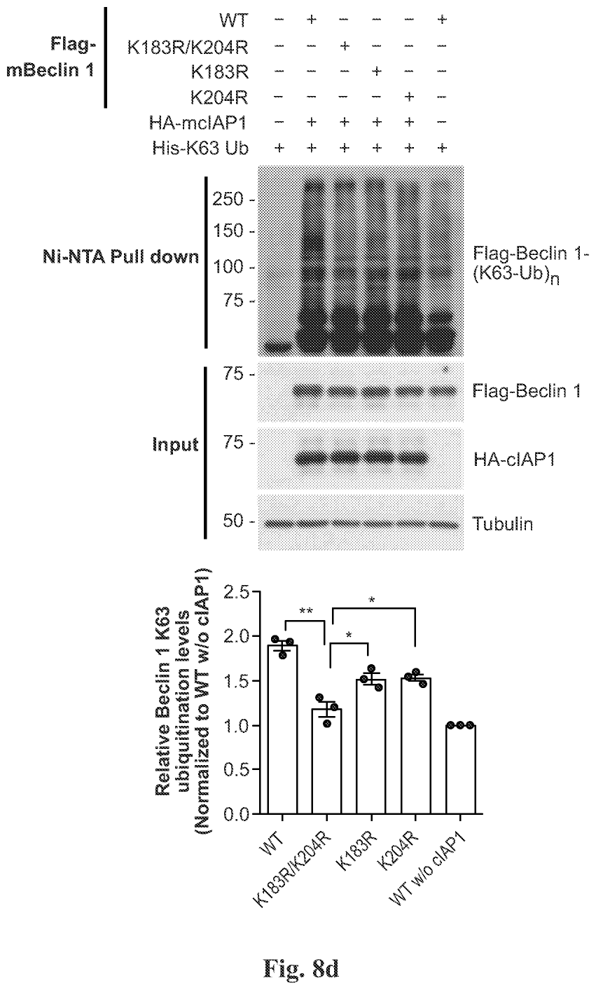

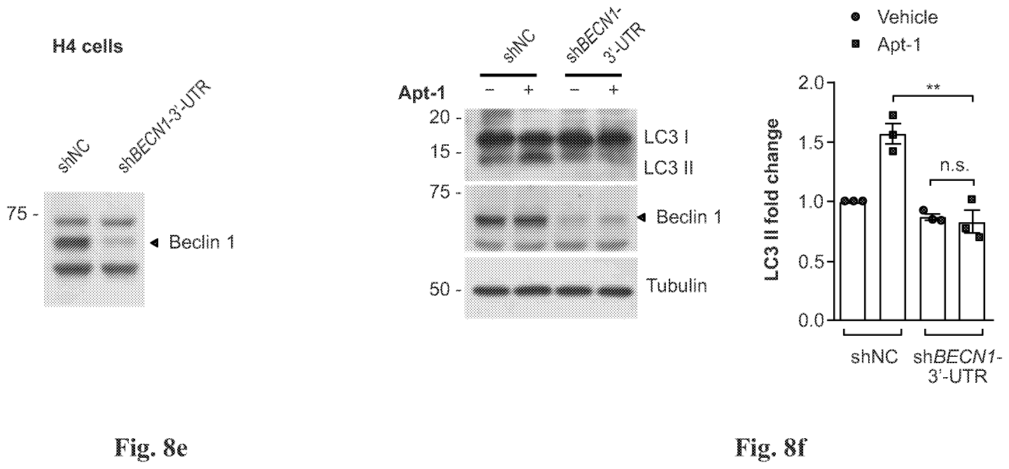

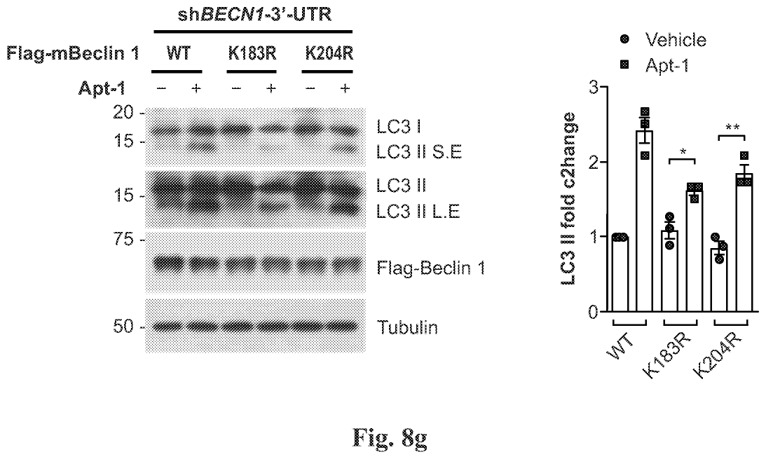

Mass spectrometry analysis identified conserved Lys183 and Lys204 in Beclin 1 that may be modified by cIAP1 (FIG. 8a-8c). Double K183R/K204R mutant, but not either single mutant, reduced K63 ubiquitination of Beclin 1 mediated by cIAP1 (FIG. 8d). Reconstitution of K183R/K204R double mutant, but not either single mutant, in Beclin 1-KD H4 cells blocked induction of autophagy (FIG. 8e-8g, FIG. 1h) and reduced K63 ubiquitination of Beclin 1 induced by Apt-1 (FIG. 1i). These results suggest that Apt-1/ICCB-19 promote autophagy via K63 ubiquitination of Beclin 1 mediated by cIAP1/2 and TRAF2.

### ICCB-19/Apt-1 Indirectly Inhibit RIPK1 Kinase

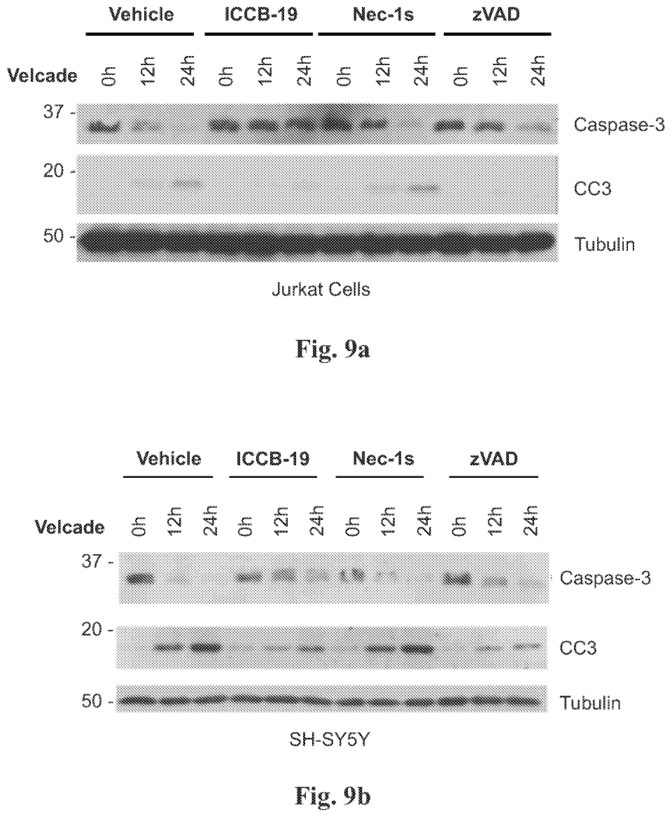

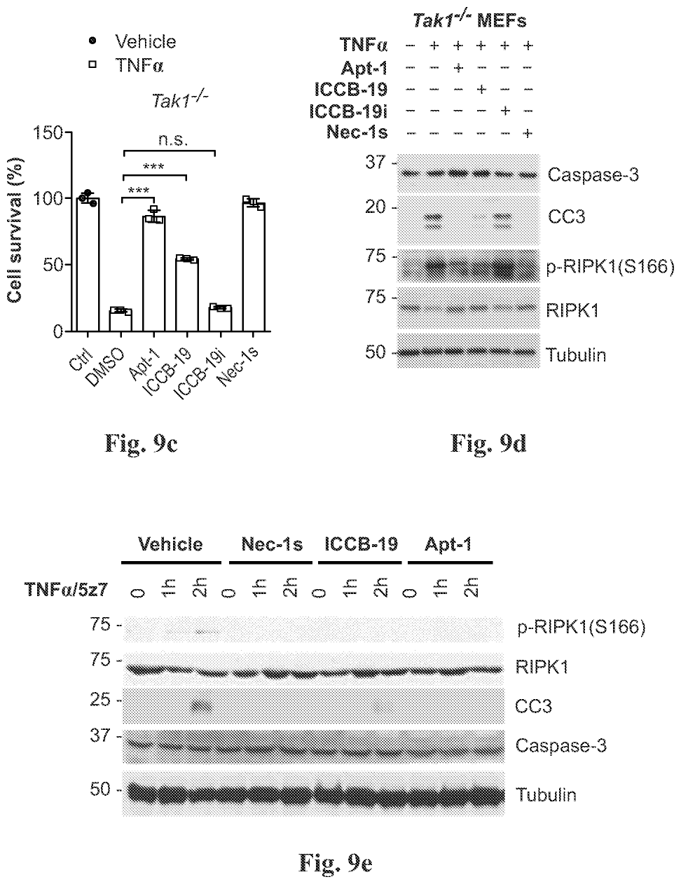

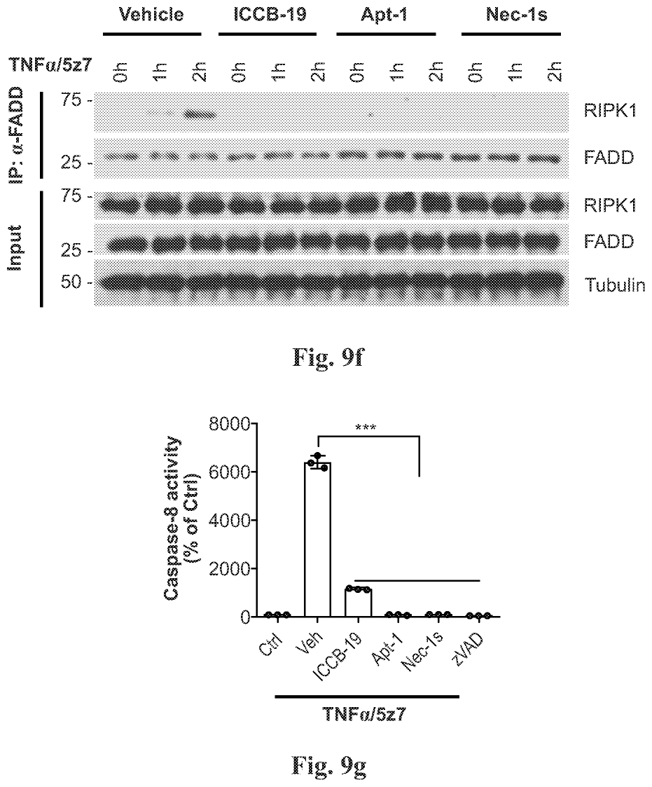

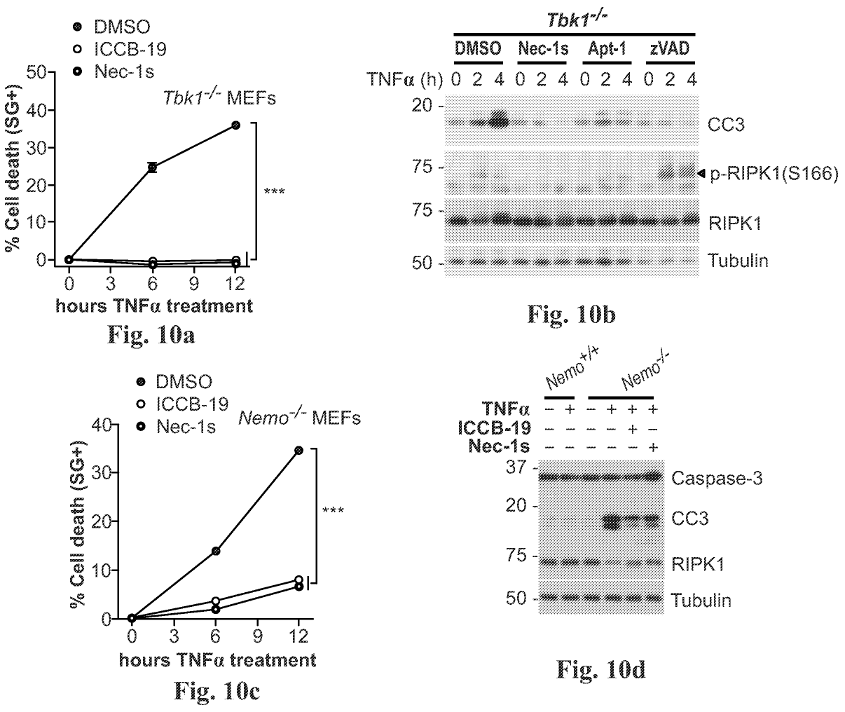

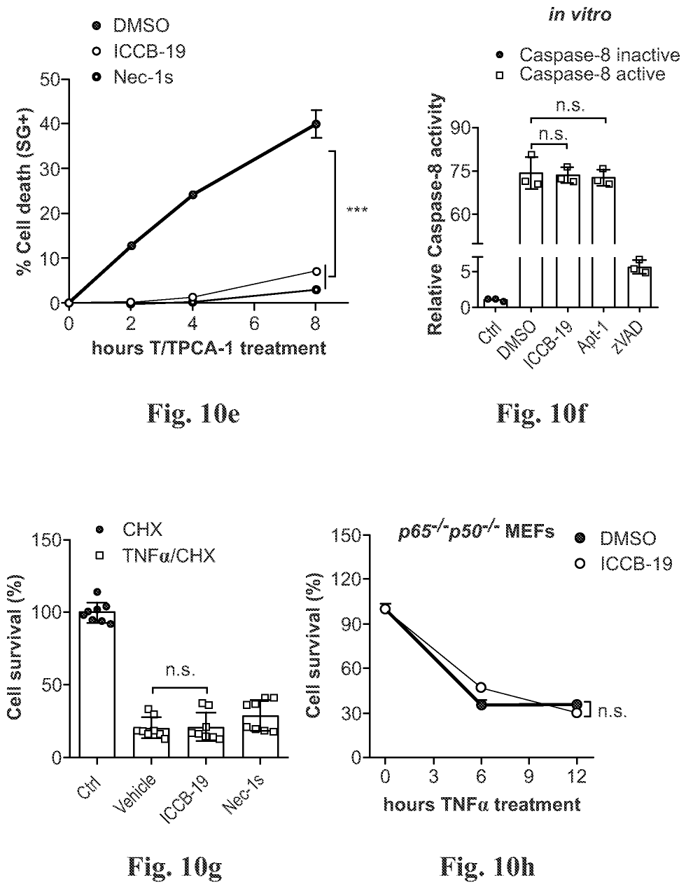

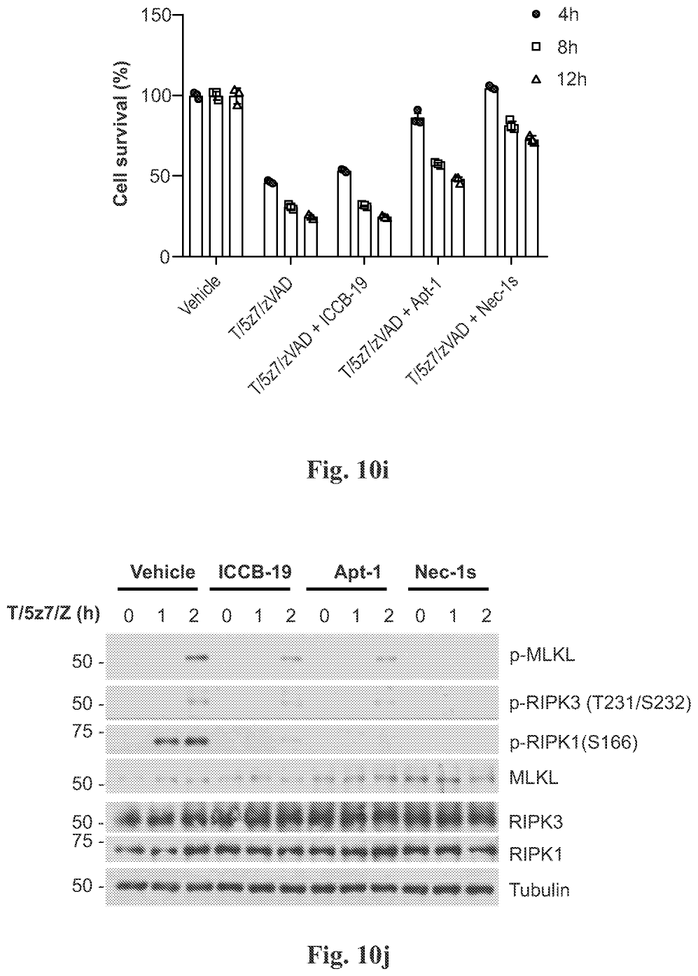

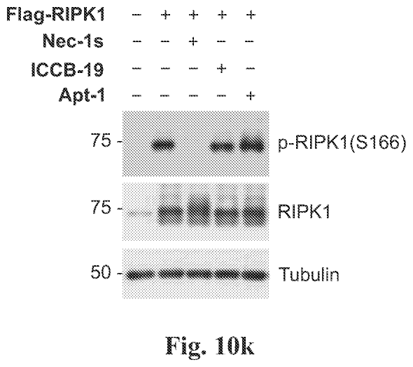

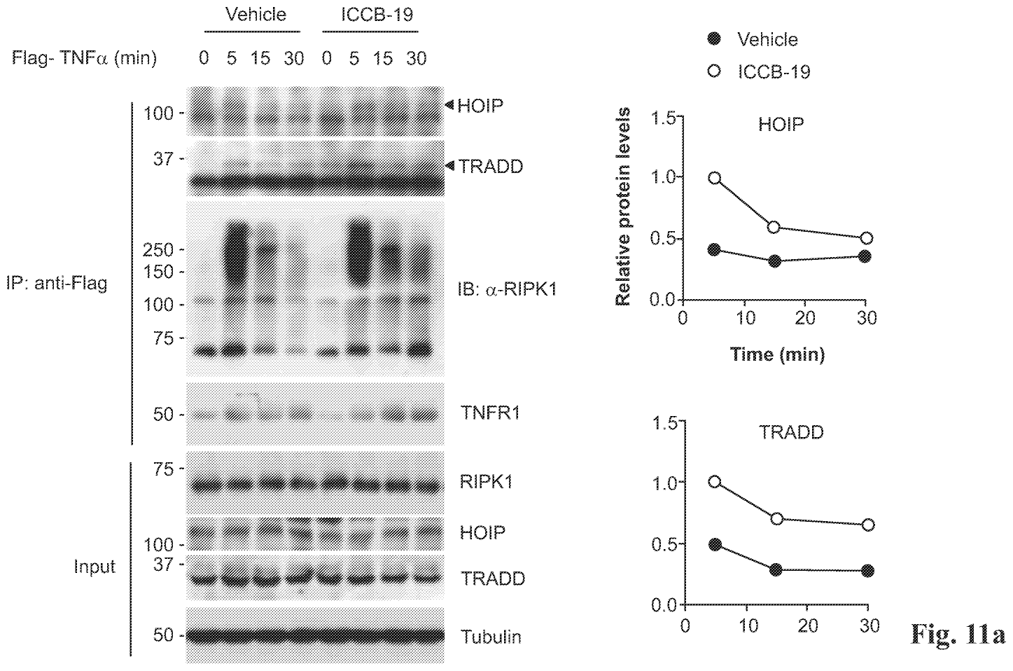

The cleavage of caspase-3 downstream of Velcade-induced proteasomal stress was blocked by ICCB-19/Apt-1, but not RIPK1 inhibitor Necrostatin-1s (Nec-1s) (FIG. 9a, 9b). Like Nec-1s, ICCB-19/Apt-1 protected against multiple RDA models (FIG. 9c-9g, 10a-10f), but not RIPK1-independent apoptosis (RIA) (FIG. 9g, 9h). ICCB-19/Apt-1 also partially inhibited necroptosis (FIG. 10i, 10j). However, unlike Nec-1s, ICCB-19/Apt-1 cannot inhibit activation of overexpressed RIPK1 (FIG. 10k). Thus, ICCB-19/Apt-1 are indirect inhibitors of RIPK1 kinase activity.

### ICCB-19/Apt-1 Require TRADD to Block Apoptosis and Activate Autophagy

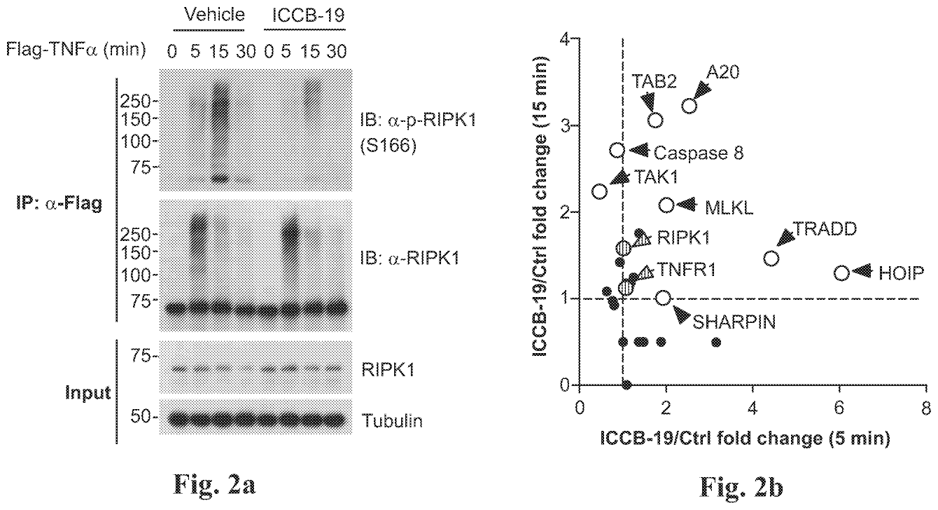

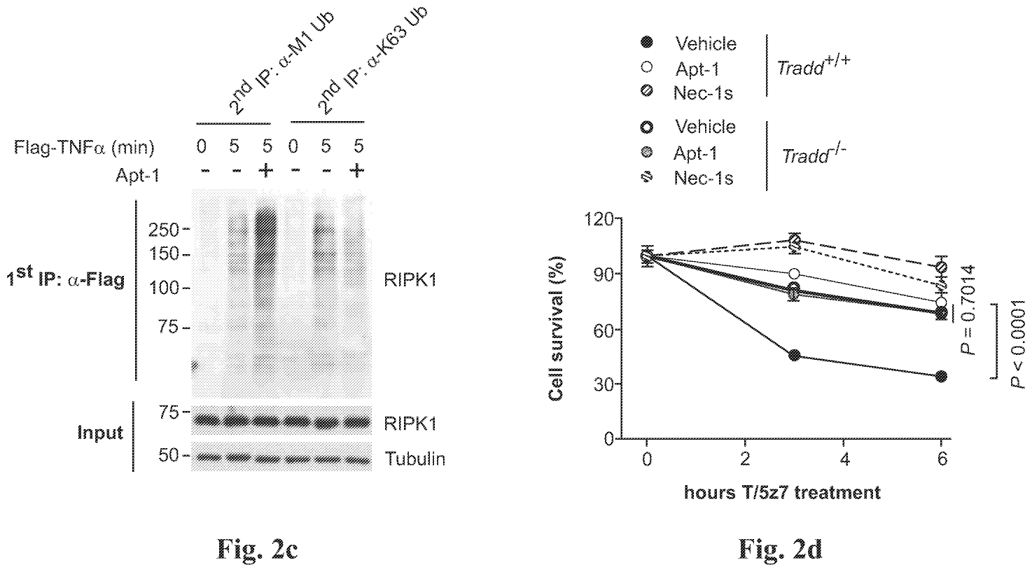

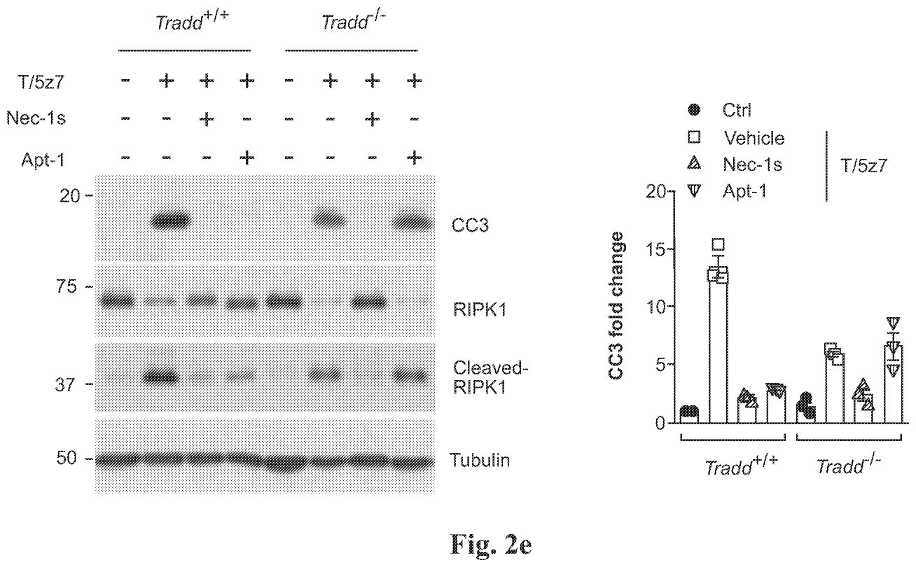

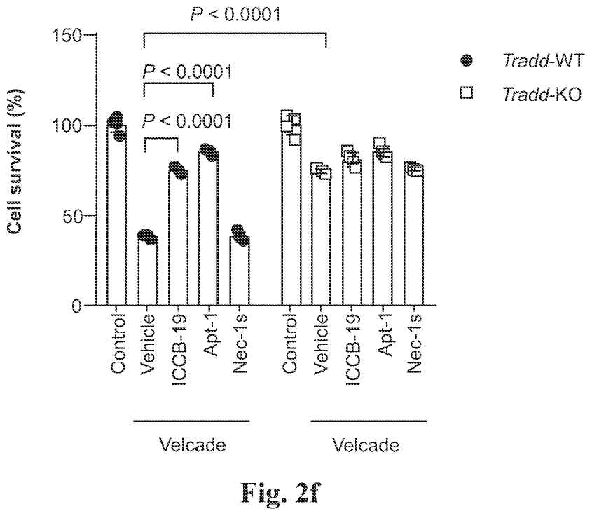

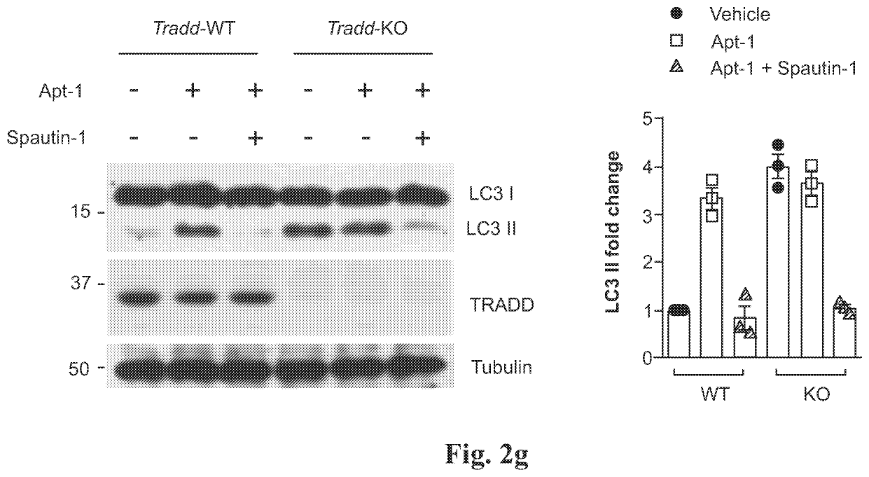

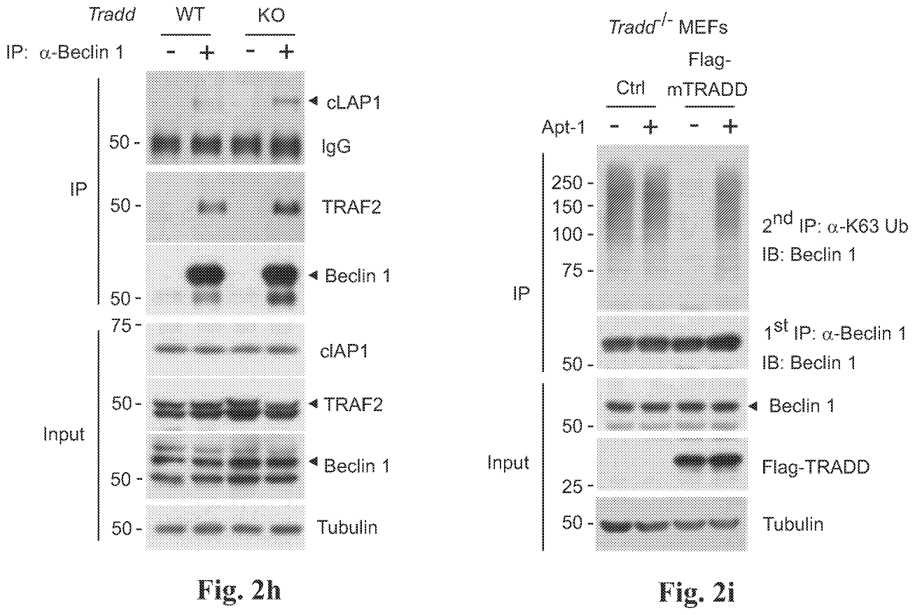

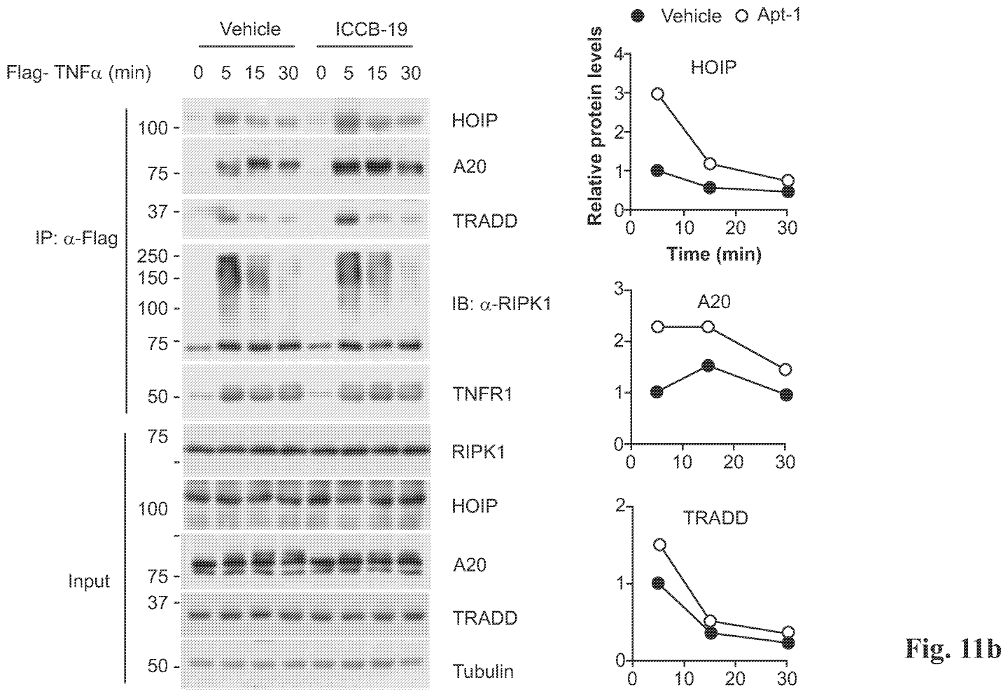

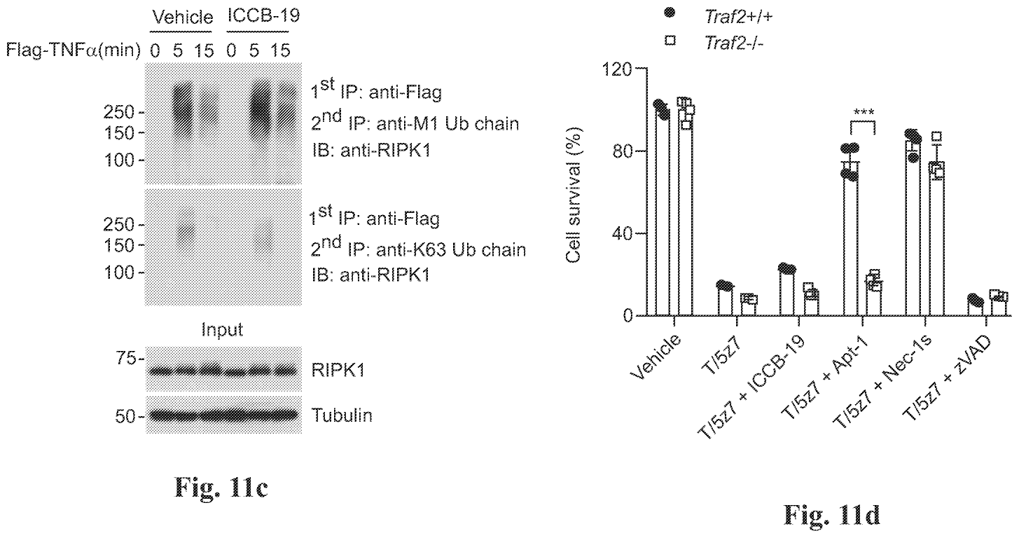

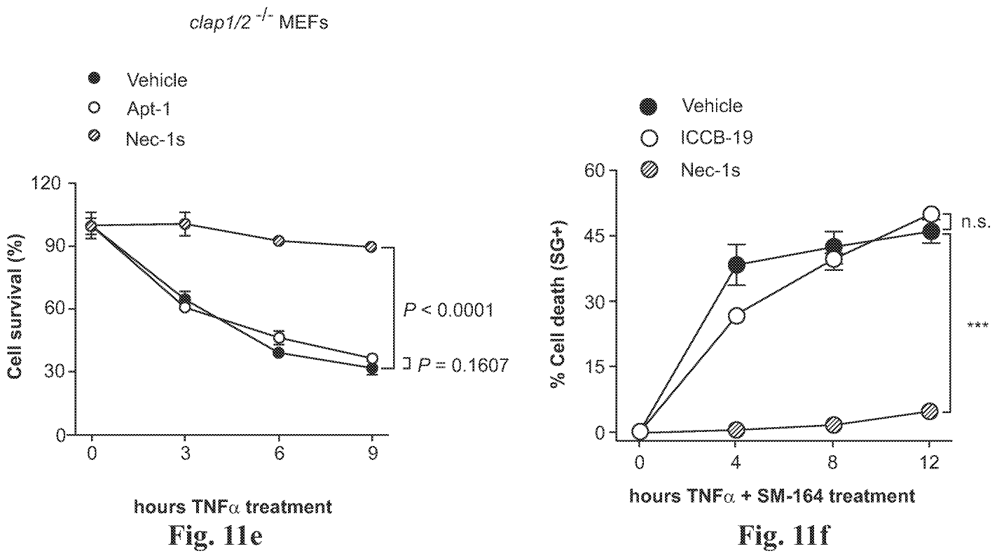

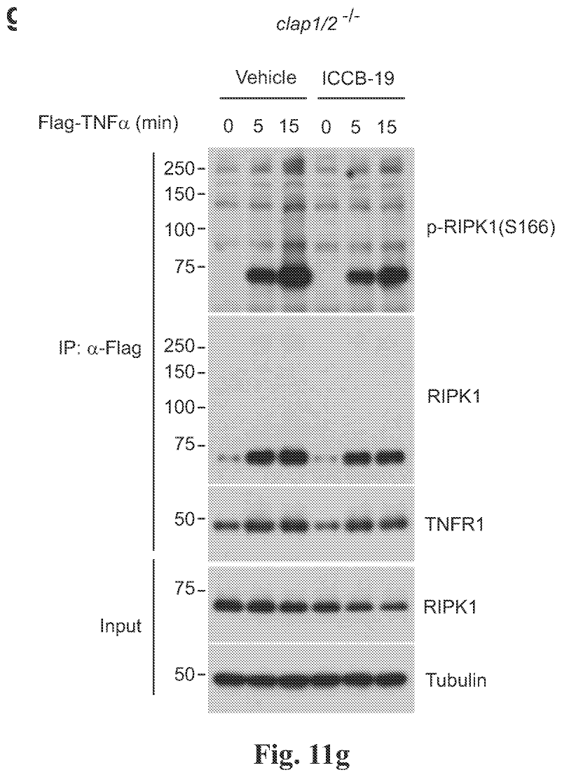

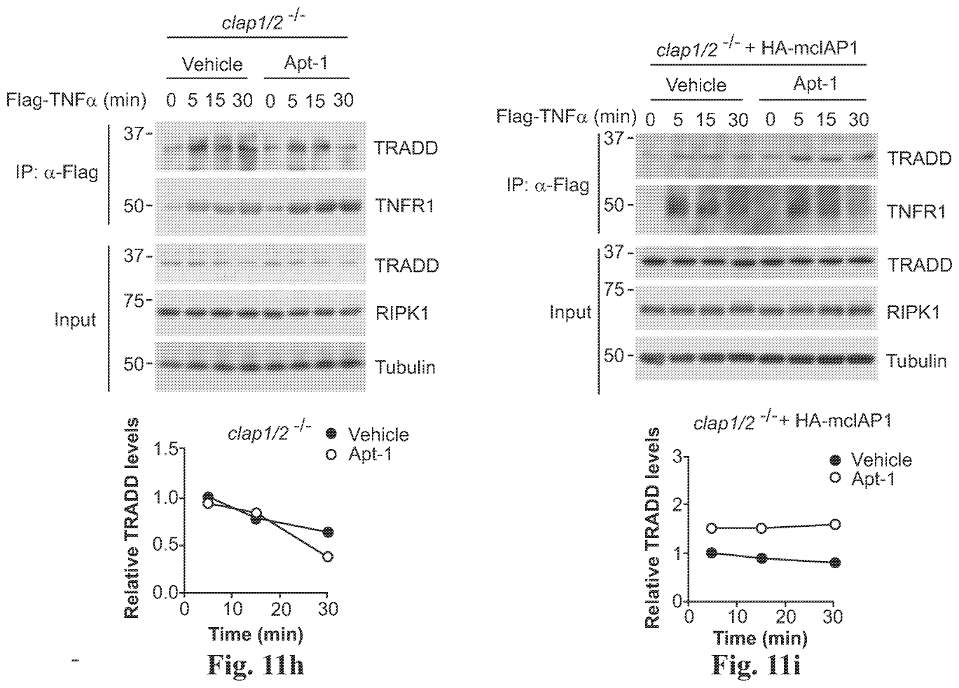

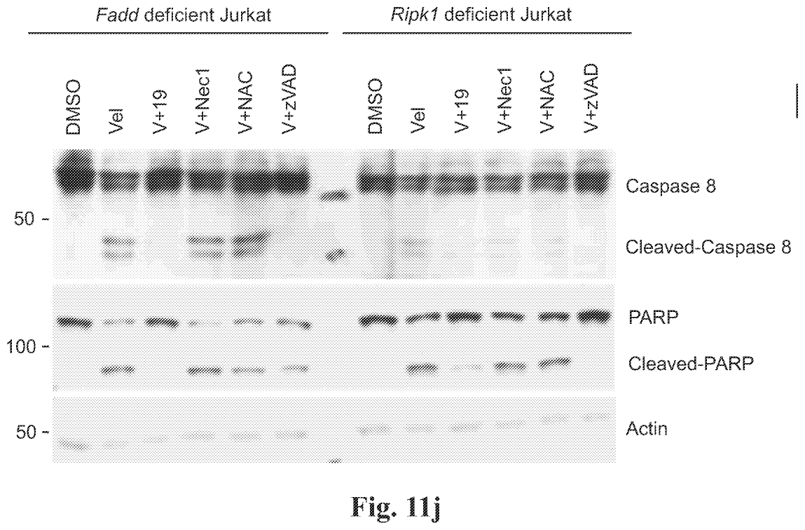

TNFα stimulation promotes the formation of a transient intracellular complex (complex I) at TNFR1 which coordinates an intricate set of ubiquitination and phosphorylation events, including both K63 ubiquitination mediated by TRAF2/cIAP1 and M1 ubiquitination mediated the LUBAC complex, to control the activation of RIPK1. ICCB-19 treatment reduced the rapid activation of RIPK1 in complex I induced by TNFα (FIG. 2a), suggesting that the target of ICCB-19/Apt-1 may be a component of complex I. Mass spectrometry analysis and immunoprecipitation-immunoblot found that ICCB-19 increased recruitment of TRADD, HOIP, and A20, but not RIPK1, in complex I (FIG. 2b; FIG. 1a, 1b). Consistently, in ICCB-19/Apt-1-treated cells, M1 ubiquitination of RIPK1 in complex I was increased whereas K63 ubiquitination was reduced (FIG. 2c, FIG. 11c).

Since TRADD (Tumor necrosis factor receptor 1-associated DEATH domain), a 34 kDa adaptor with an N-terminal TRAF2 binding domain and C-terminal death domain, is the first protein recruited to complex I, these results suggest that TRADD may be the target for ICCB-19/Apt-1. Tradd^{-/-} MEFs are known to be resistant to RDA. Interestingly, in Tradd^{-/-} -MEFs Nec-1s, but not ICCB-19/Apt-1, offered additional protection against RDA (FIG. 2d, 2e). Thus, TRADD is required for protection of RDA by ICCB-19/Apt-1, but not Nec-1s.

In complex I, the N-terminal TRAF2-binding domain of TRADD (TRADD-N) interacts with TRAF2 and cIAP1/2 to promote the K63 ubiquitination of RIPK1. Consistently, the protective effects of ICCB-19/Apt-1, but not Nec-1s, against RDA were reduced by genetic or pharmacological inhibition of cIAP1/2 and restored by reconstitution of cIAP1 (FIG. 11d-11i). Thus, cIAP1/2-mediated ubiquitination is involved in stabilizing TRADD in complex I and suppressing activation of RIPK1 in cells treated with ICCB-19/Apt-1.

Tradd-knockout Jurkat cells were effectively protected against Velcade-induced apoptosis, which cannot be further enhanced by ICCB-19/Apt-1 (FIG. 2f), confirming that protection against Velcade-induced apoptosis by ICCB-19/Apt-1 requires TRADD, but does not require FADD or RIPK1 (FIG. 1j, 1k).

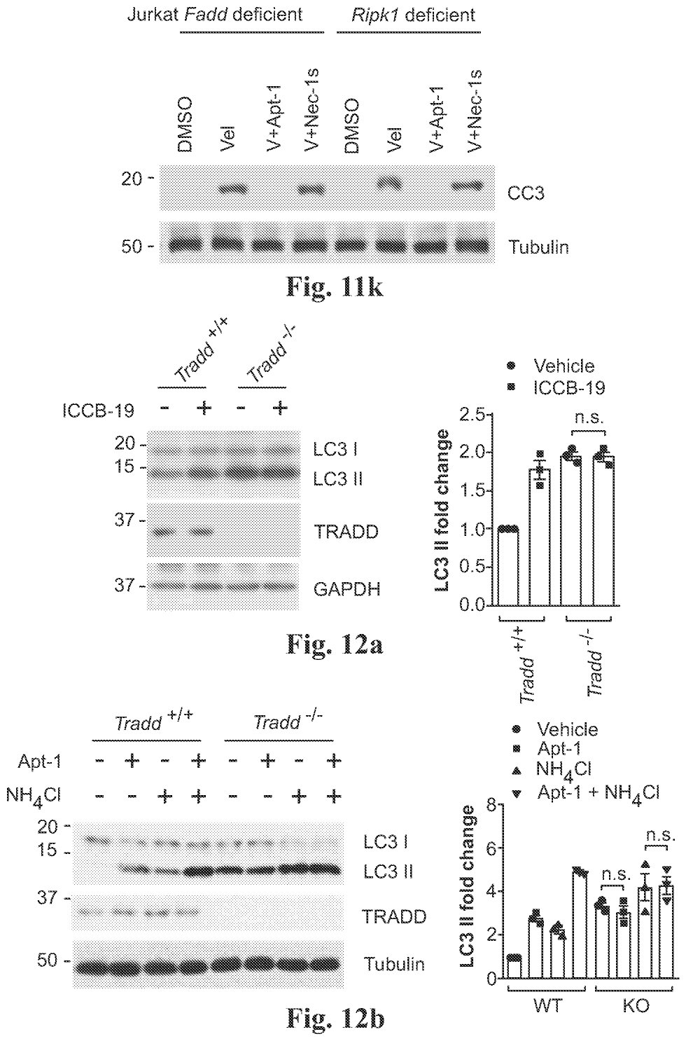

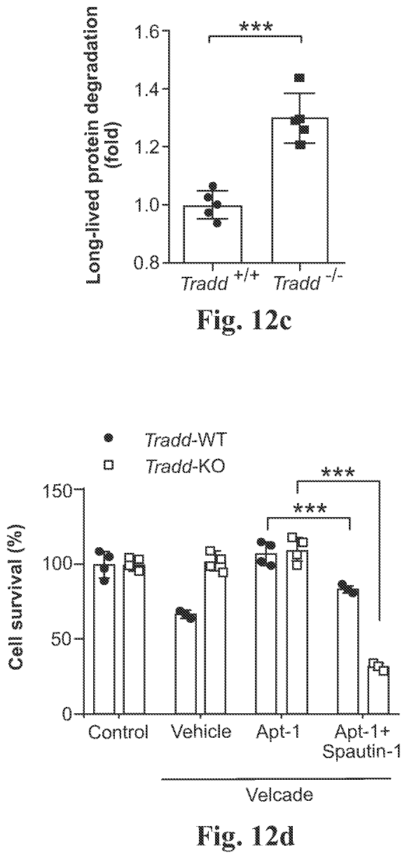

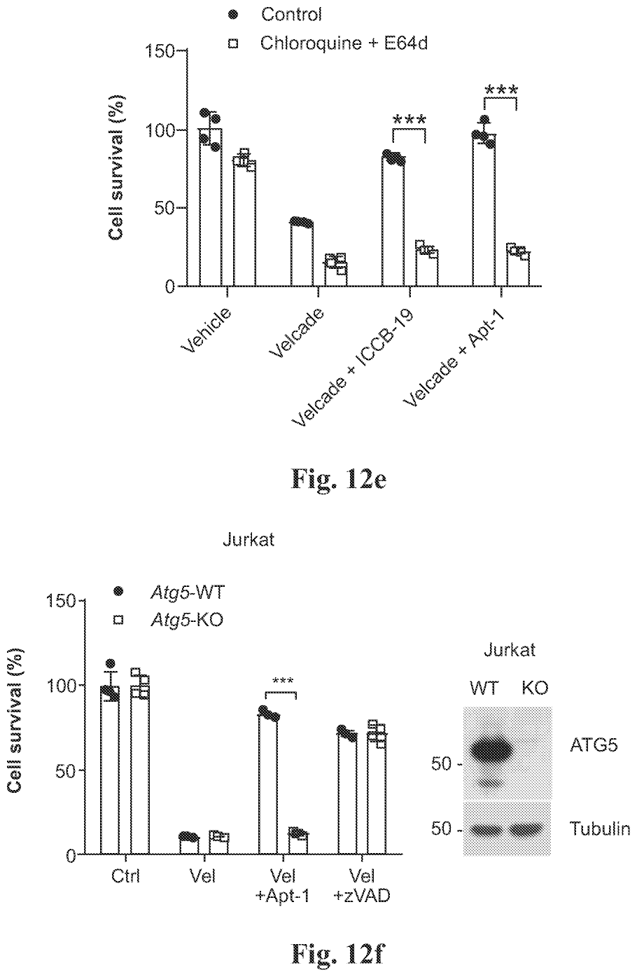

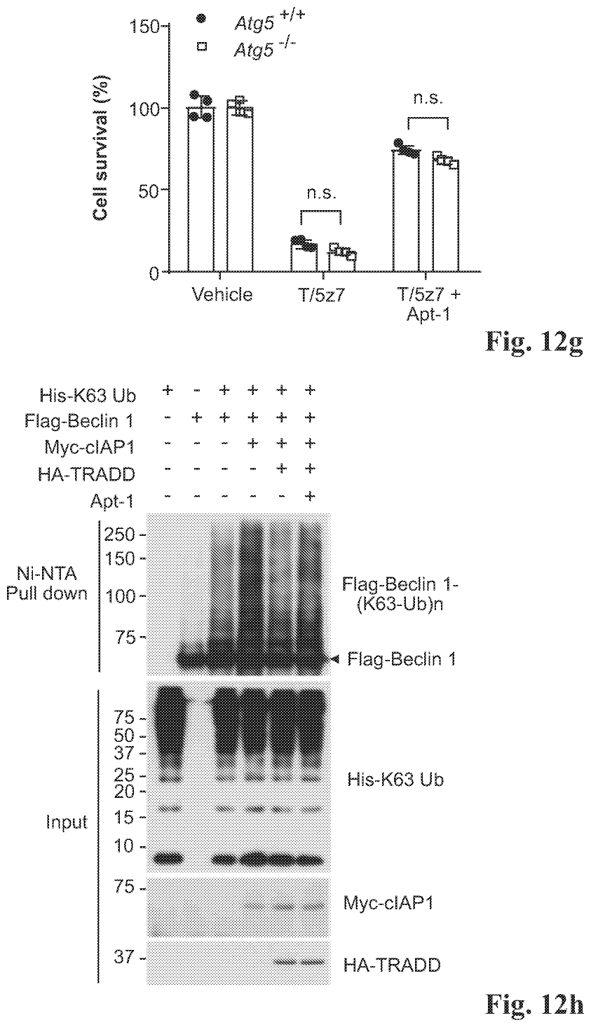

In Tradd^{-/-} MEFs basal autophagic flux and long-lived protein degradation was increased compared to that of WT, which could not be further enhanced by ICCB-19/Apt-1 (FIG. 12a-12c). Tradd-knockout Jurkat cells also displayed increased levels of LC3II, which was not altered by Apt-1 (FIG. 2g). Thus, TRADD is involved in mediating autophagy and required for ICCB-19/Apt-1-mediated induction of autophagy. Inhibition of autophagy by Spautin-1, Atg5 knockout, or blocking lysosomal degradation prevented autophagy activation and protection against proteasomal stress-induced apoptosis in Tradd knockout or Apt-1, but had no effect on RDA (FIG. 2g, FIG. 12d-12g).

Under homeostatic conditions, endogenous Beclin 1 bound to cIAP1/2 and TRAF2; this binding was enhanced by TRADD deficiency (FIG. 2h). Furthermore, K63 ubiquitination of Beclin 1 was enhanced in Tradd^{-/-} MEFs, which was not affected by addition of Apt-1, but could be suppressed by adding back TRADD; this suppression was overcome by treatment with Apt-1 (FIG. 2i; FIG. 12h). Taken together, these results suggest that ICCB-19/Apt-1 promote cIAP1/2/TRAF2-mediated K63 ubiquitination of Beclin 1 by releasing cIAP1/2/TRAF2 from their endogenous interactions with TRADD.

### ICCB-19/Apt-1 Reduce Inflammatory Responses

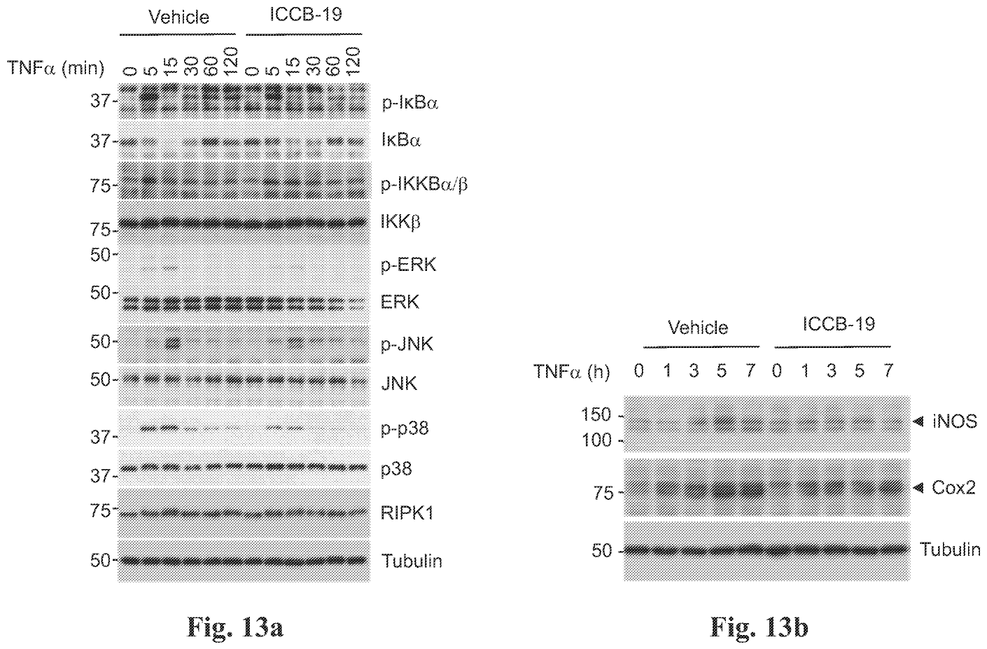

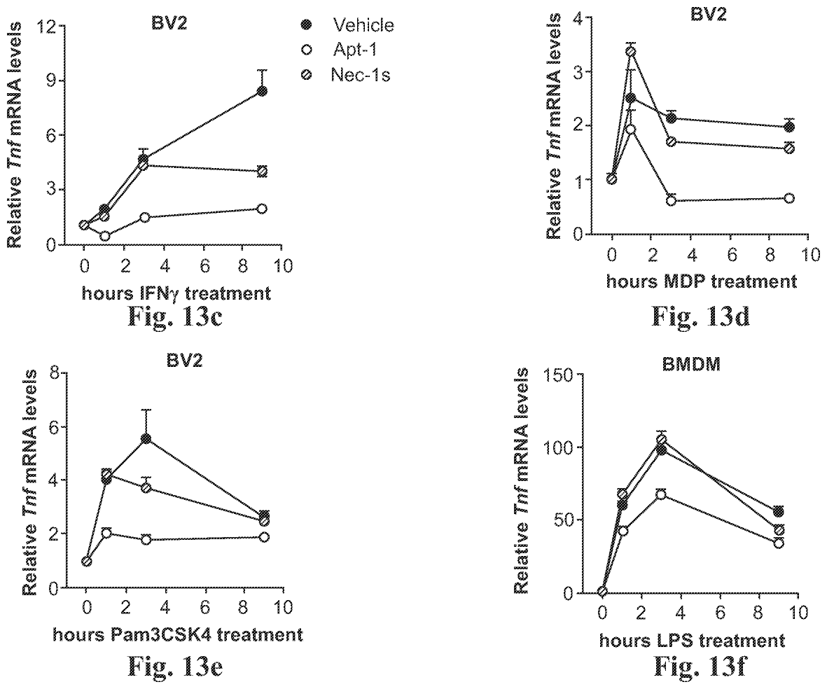

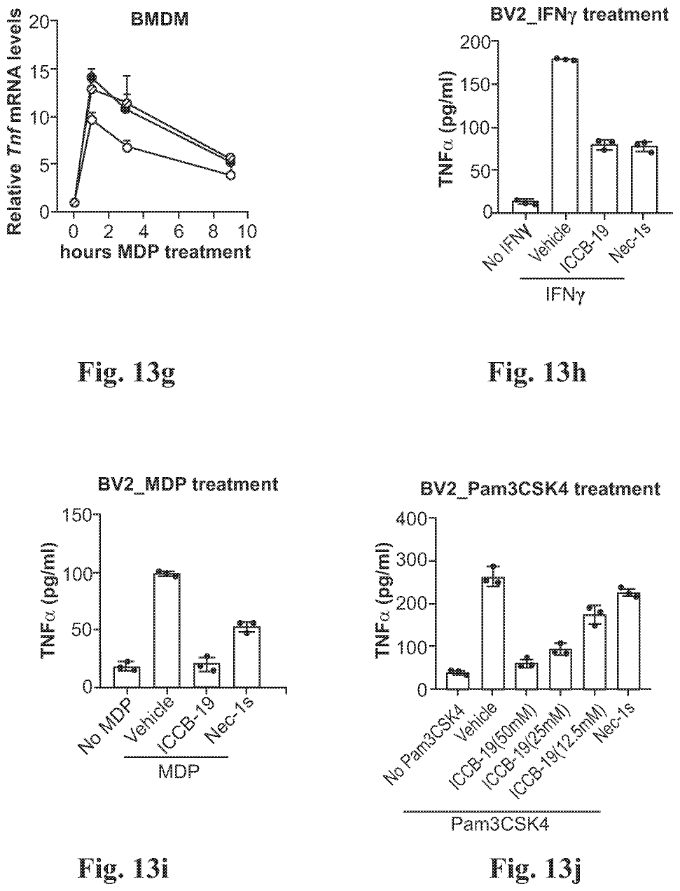

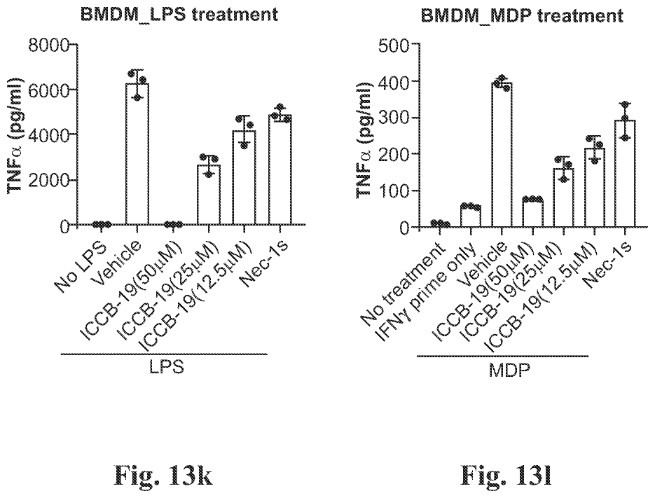

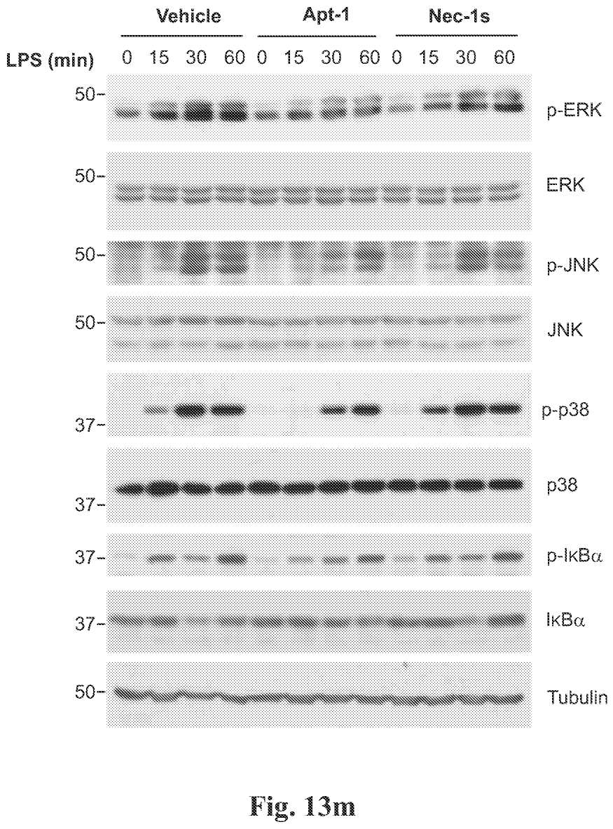

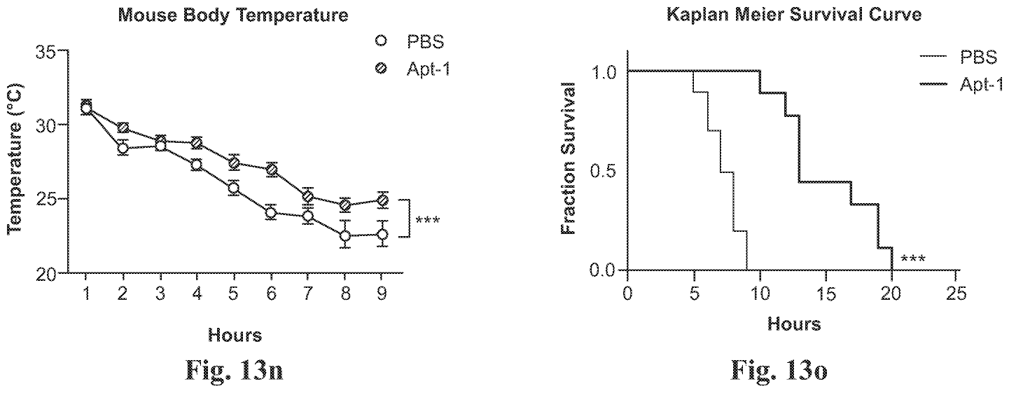

Tradd^{-/-} mice are normal throughout development and adulthood and are highly resistant to multiple systemic inflammatory responses. Treatment with ICCB-19/Apt-1 minimally affected early events in the NF-κB pathway, but reduced production of TNFα-induced inflammatory target genes, NOS and COXII and inflammatory cytokines in cells stimulated with pathogen-associated molecular patterns (PAMPs), including interferon γ (IFNγ), lipopolysaccharide (LPS), Pam3CSK4 (a synthetic bacterial lipopeptide), or muramy1 dipeptide (MDP) (FIG. 13a-13m). Consistently, WT mice treated with Apt-1 showed increased survival following intravenously-delivered TNFα, a murine model of systemic inflammation (FIG. 13n, 130).

### ICCB-19/Apt-1 Restore Proteome Homeostasis

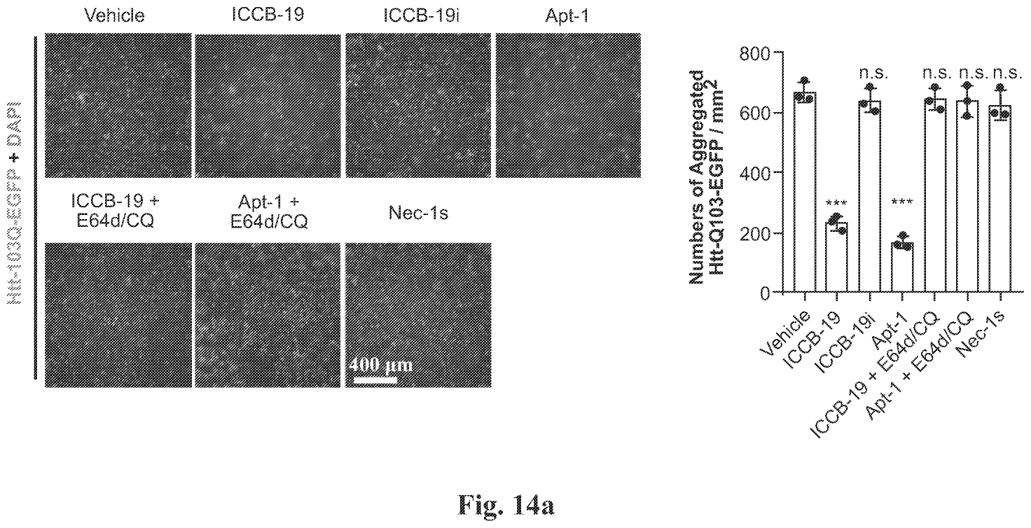

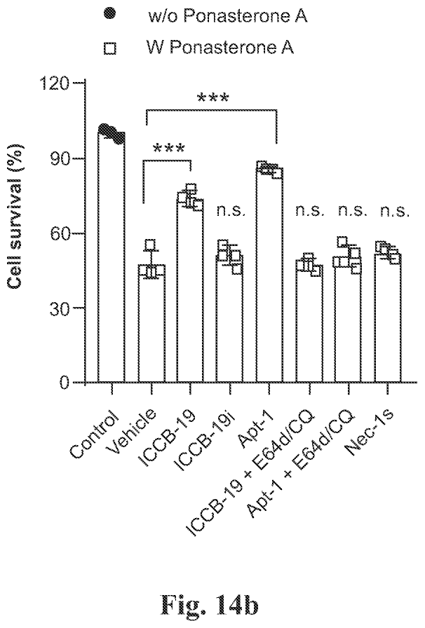

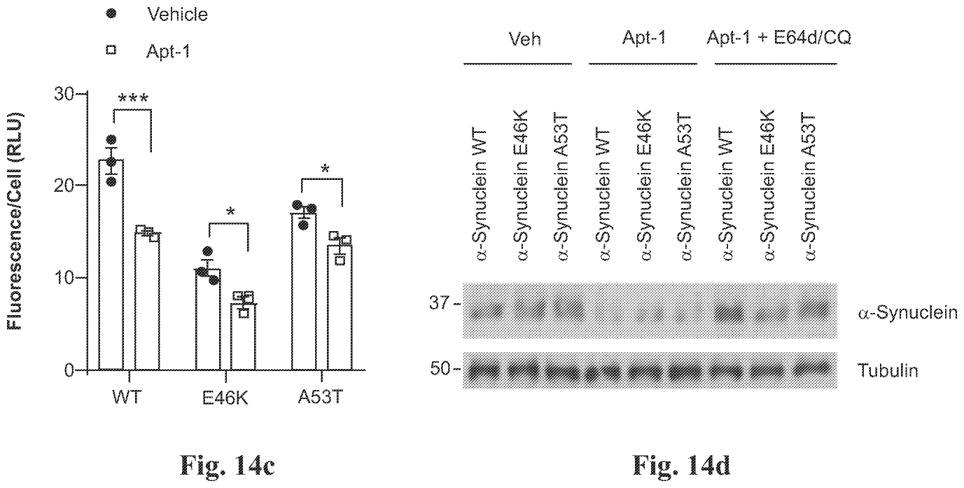

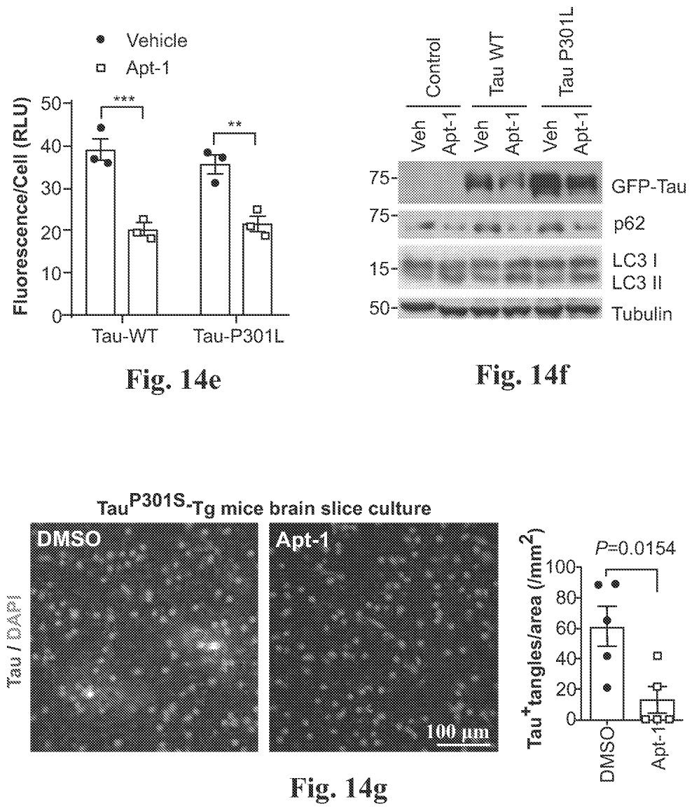

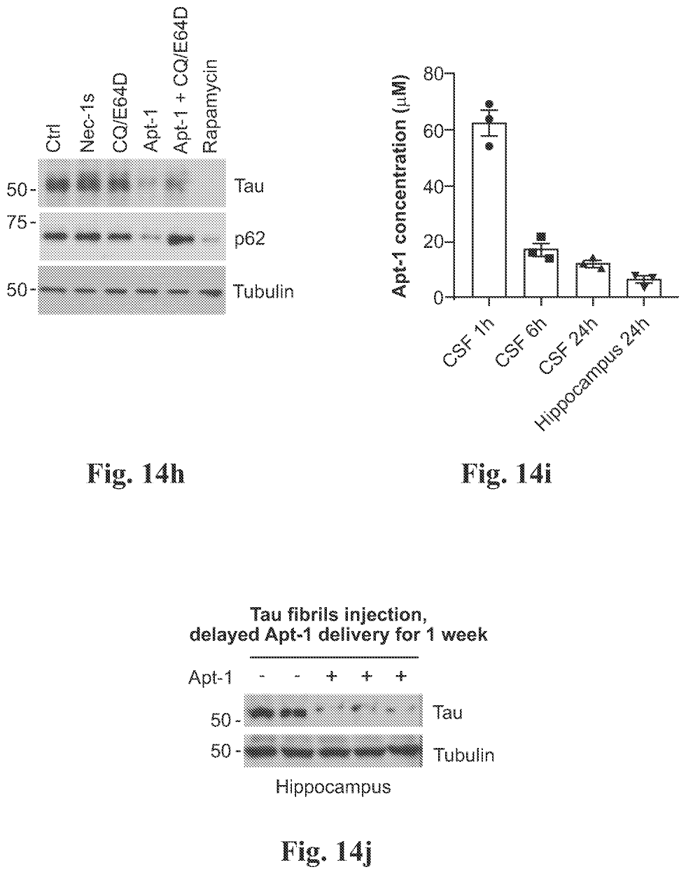

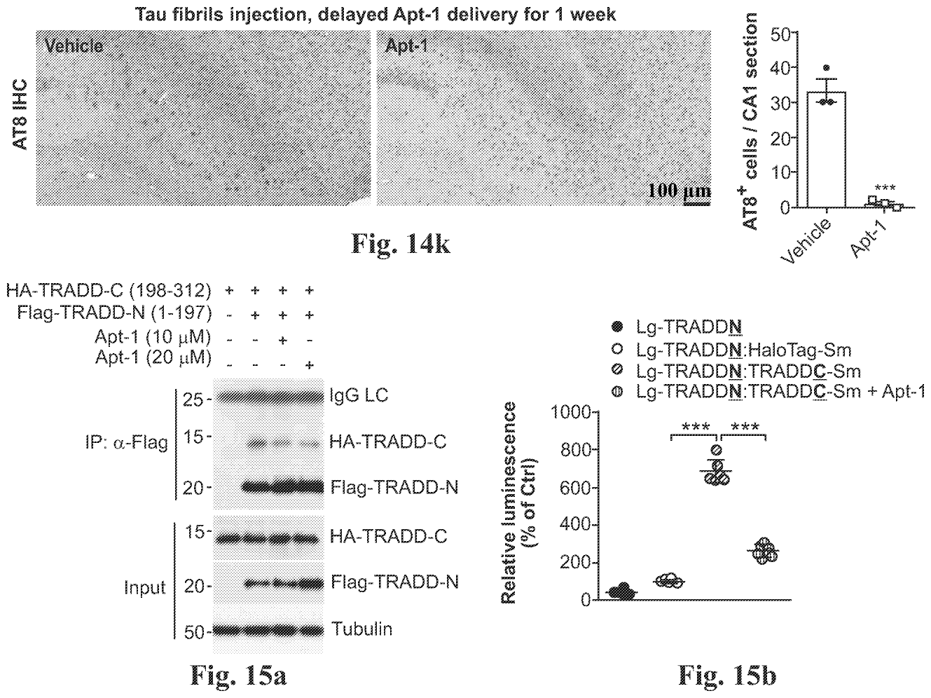

We next investigated whether ICCB-19/Apt-1 could restore cellular homeostasis and promote degradation of misfolded proteins. Treatment with ICCB-19/Apt-1 reduced protein accumulation and cell death in the presence of Htt-103Q, WT, E46K, or A53T α-synuclein, and WT or P301L tau (FIG. 14a-14f).

PS19 mice, expressing mutant hP301S tau, develop progressive neuronal loss and microgliosis associated with neurofibrillary tangle-like tau pathology. Treatment with Apt-1 for 3 h induced autophagy and reduced the accumulation of mutant tau in cultured brain slices from PS19 mice, which was blocked by lysosomal inhibition (FIG. 14g, 14h). Thus, Apt-1 can rapidly promote the degradation of accumulated mutant tau.

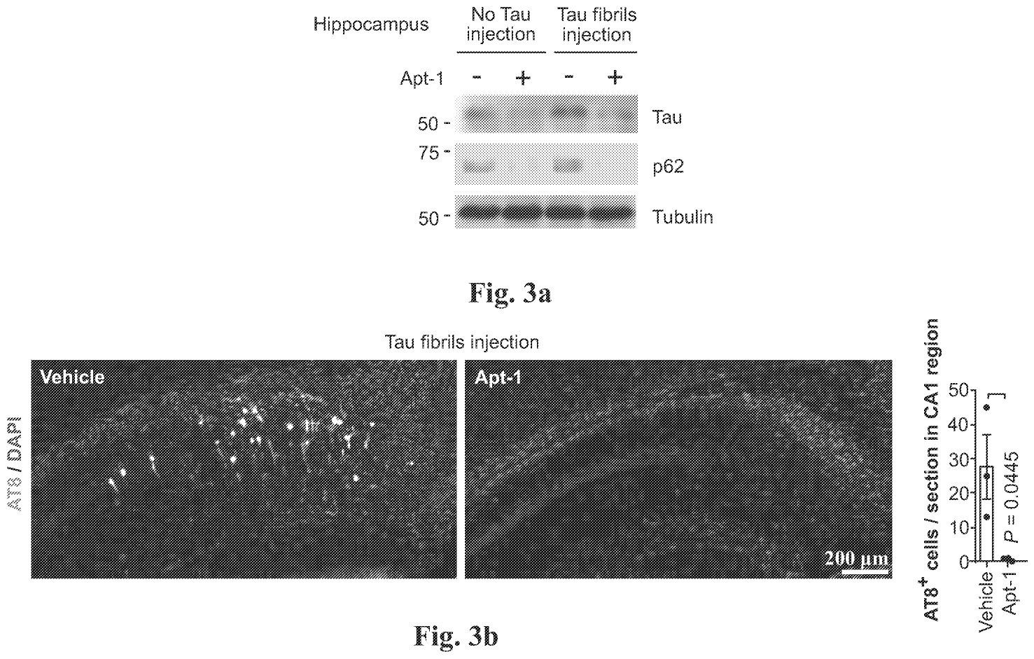

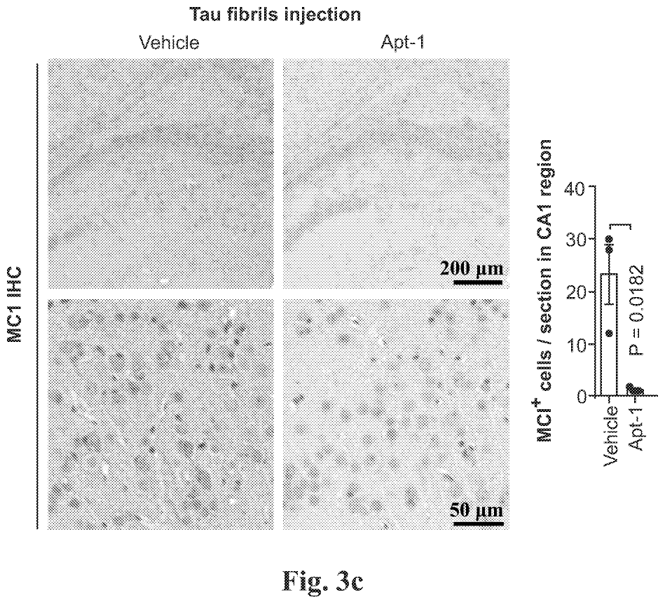

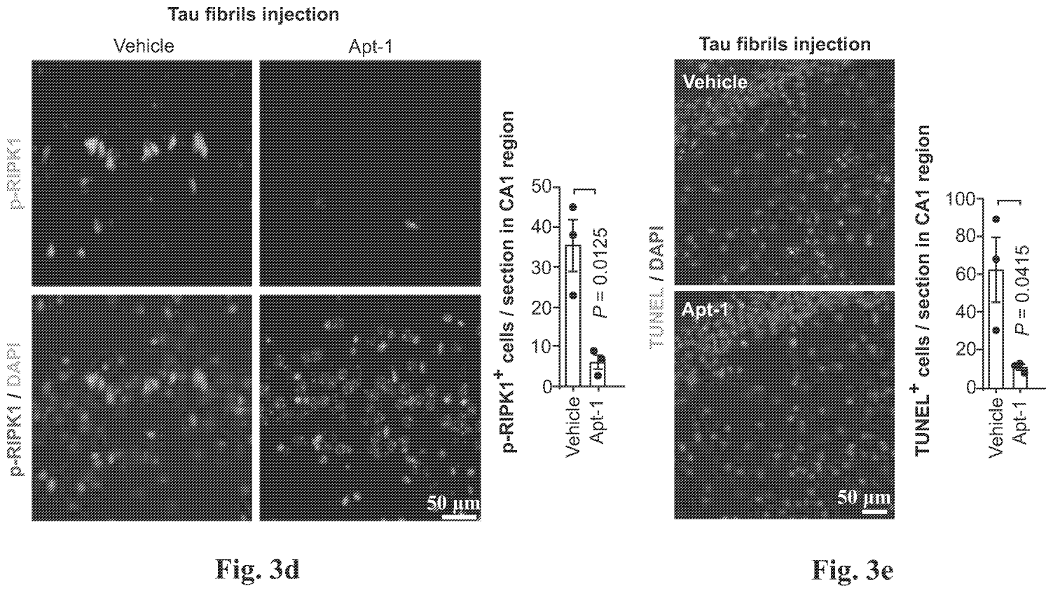

We tested the effect of Apt-1 in restoring proteostasis and reducing cell death in a mutant tau fibril (pff) transmission model. Hippocampal delivery of Apt-1 (FIG. 14i) was able to induce autophagy and reduce the levels of tau in these mice (FIG. 3a). Treatment with Apt-1 reduced neurofibrillary tangle-like pathogenesis induced by pffs, including a reduction of hyperphosphorylated tau positive neurons and levels of pathological misfolded MC1⁺ tau (FIG. 3b, 3c). Tau pff-injected mice showed substantial increases in activated pRIPK1⁺ and apoptotic TUNEL⁺ cells in the CA1 hippocampus, which was inhibited by Apt-1 (FIG. 3d, 3e). These results suggest that RIPK1 is activated in this tauopathy model and treatment with Apt-1 can effectively restore cellular homeostasis and block apoptosis driven by pathological tau transmission.

Additionally, we tested whether Apt-1 could rescue proteostasis after tangle-like pathology had formed in PS19 mice. PS19 mice were injected with pffs allowing tangle-like pathology to form for 3 weeks, and then treated with Apt-1 for 1 week which was also able to effectively reduce the accumulation of tangle-like tau aggregates (FIG. 14j, 10k).

ICCB-19/Apt-1 Interact with TRADD-N

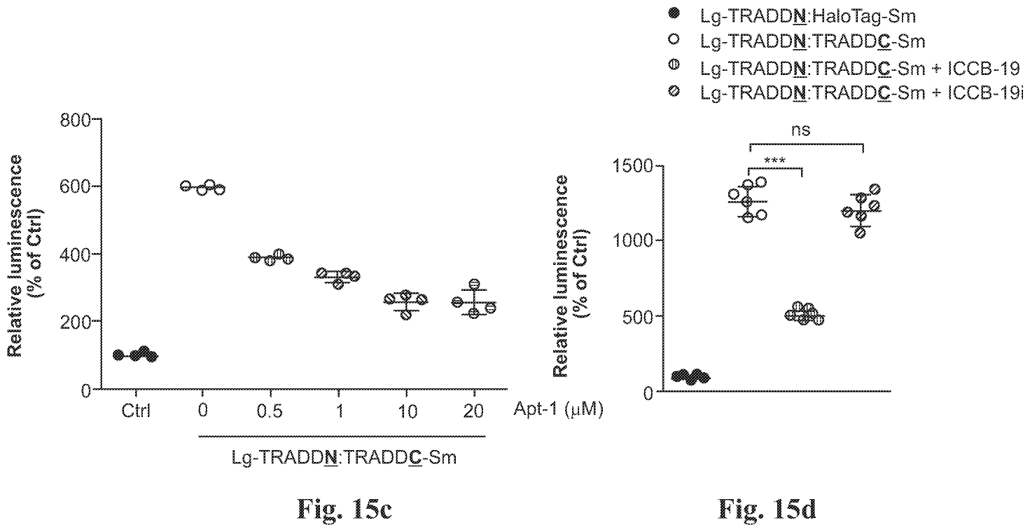

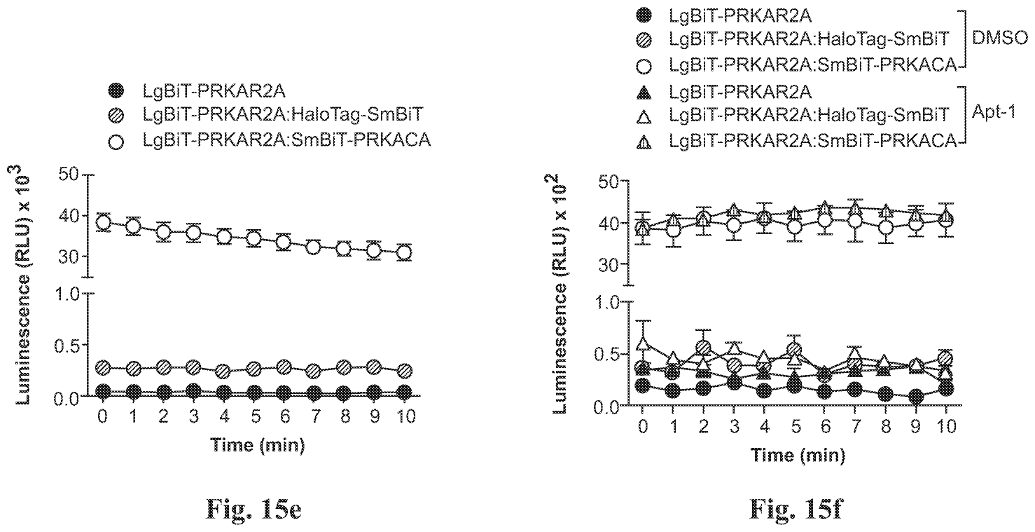

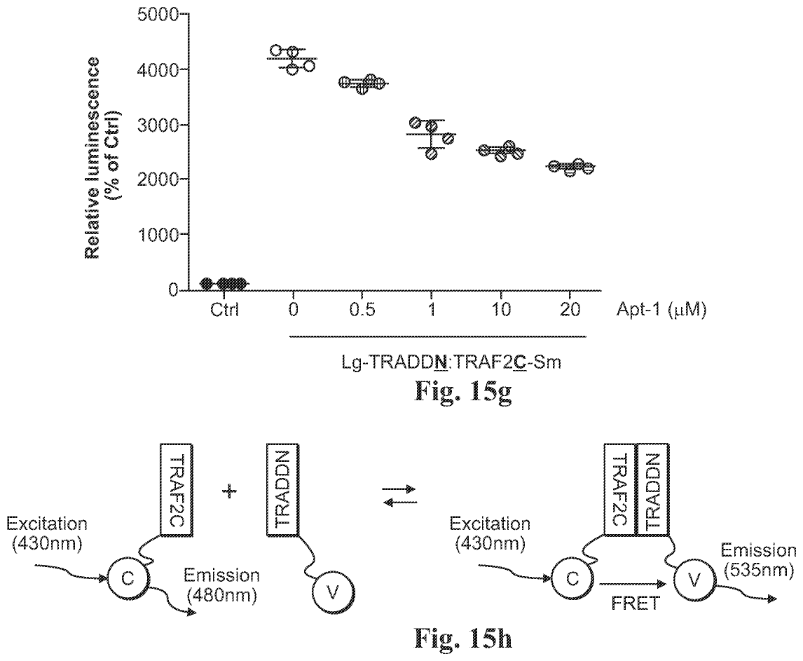

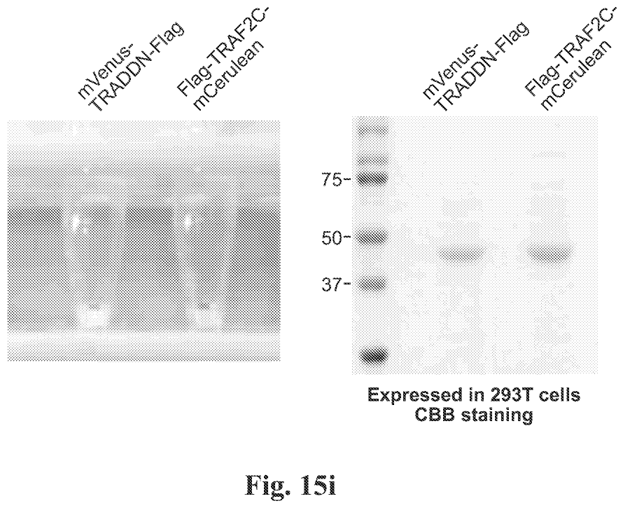

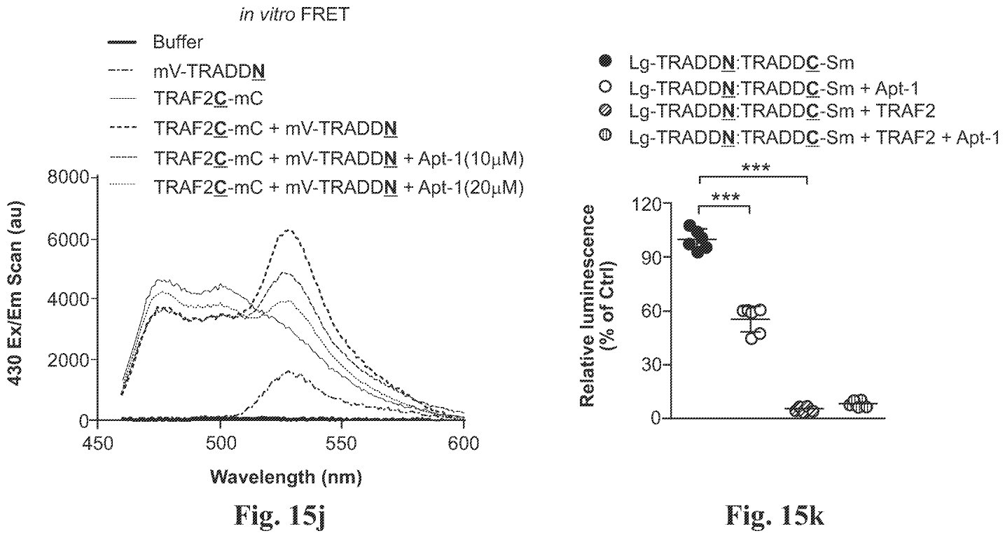

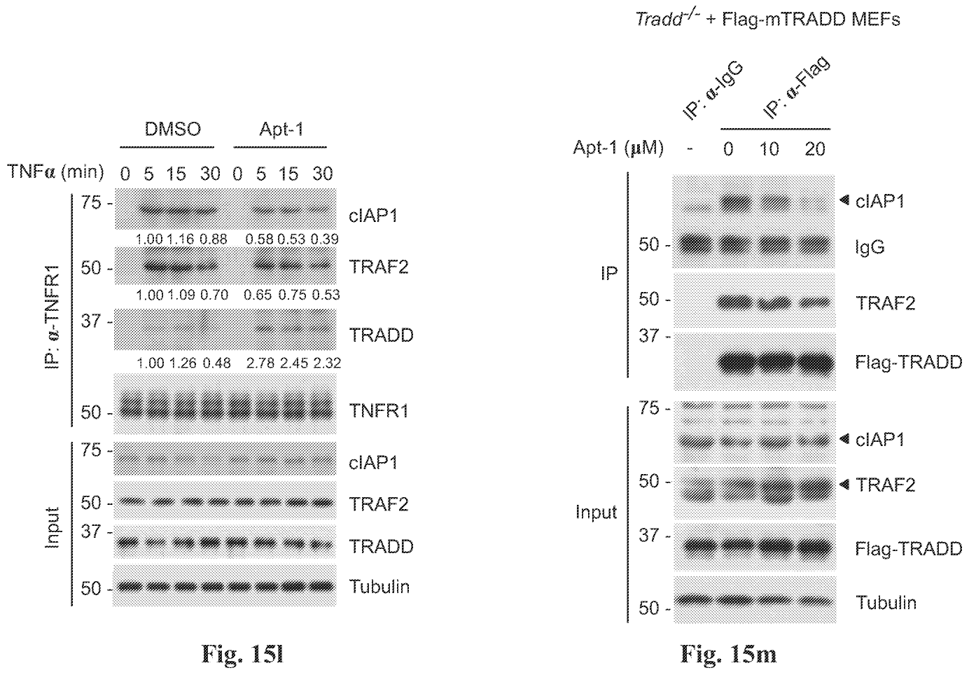

We detected interaction between separately expressed TRADD-N(a.a.1-197) and TRADD-C (a.a.198-312), which was reduced in cells treated with Apt-1 (FIG. 15a), and thus, ICCB-19/Apt-1 might affect a previously unknown interaction of TRADD-N with TRADD-C. NanoBit-based interaction signal between LgBiT-TRADD-N and TRADD-C-SmBiT was dose-dependently reduced by ICCB-19/Apt-1, but not ICCB-19i (FIG. 15b-15f).

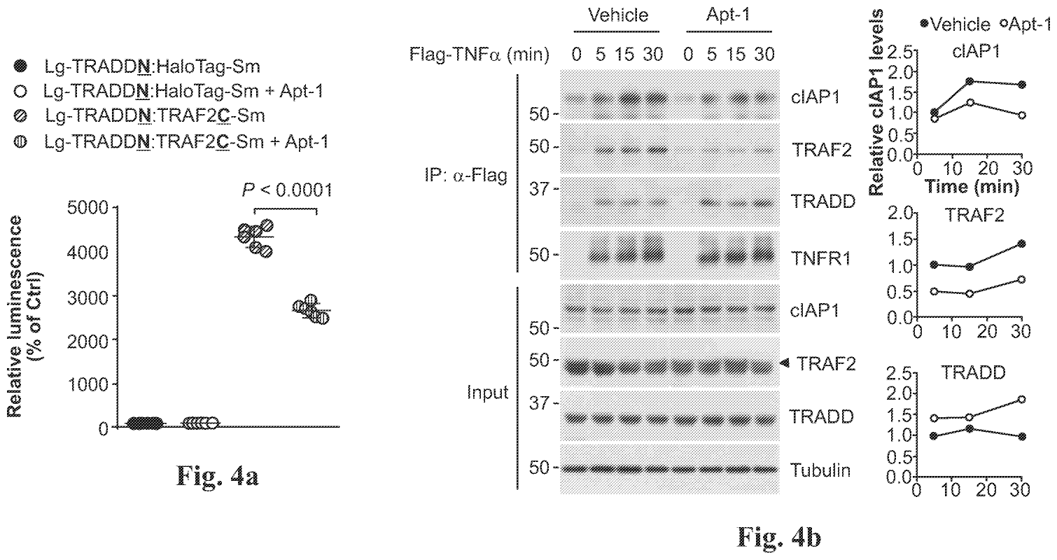

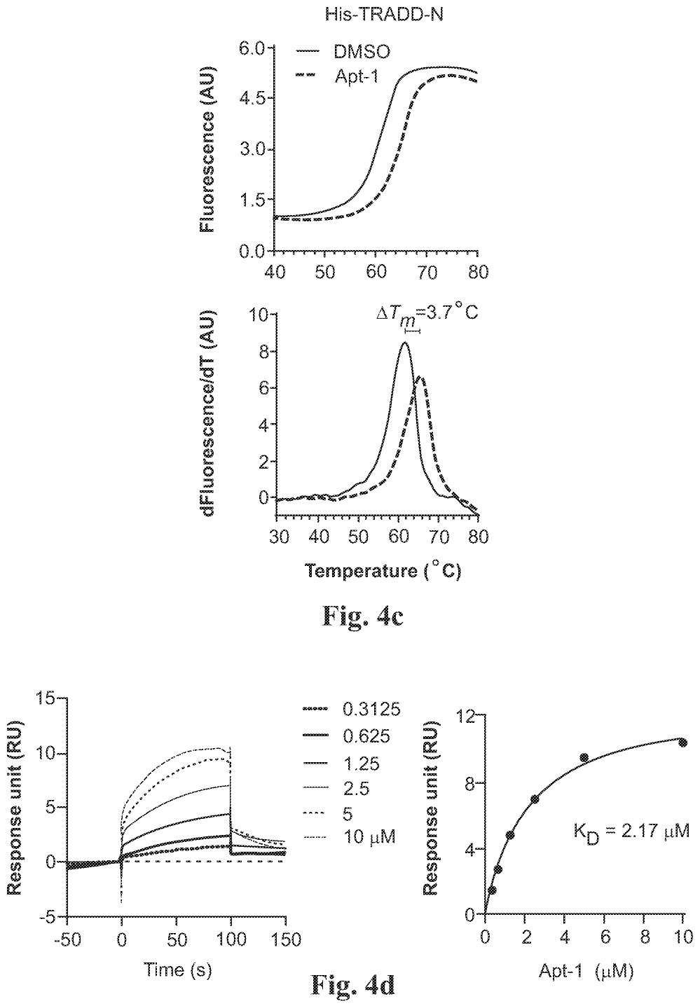

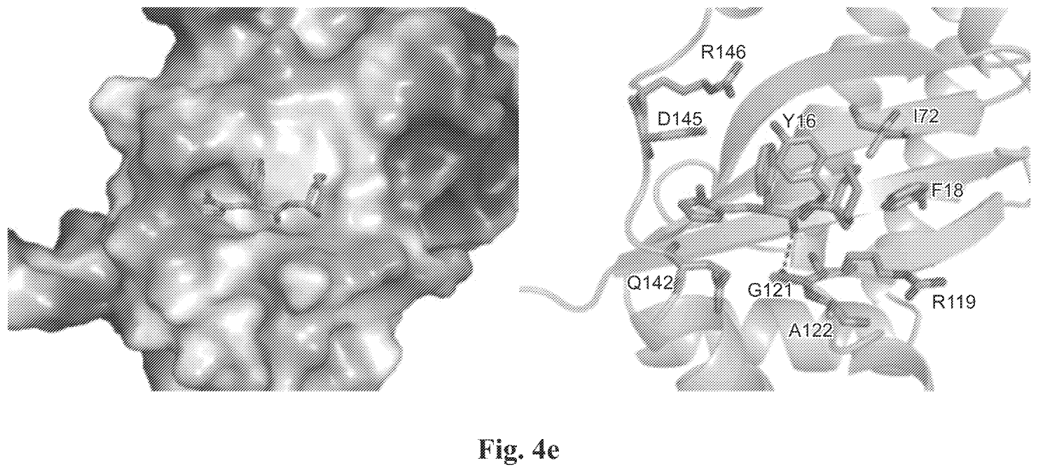

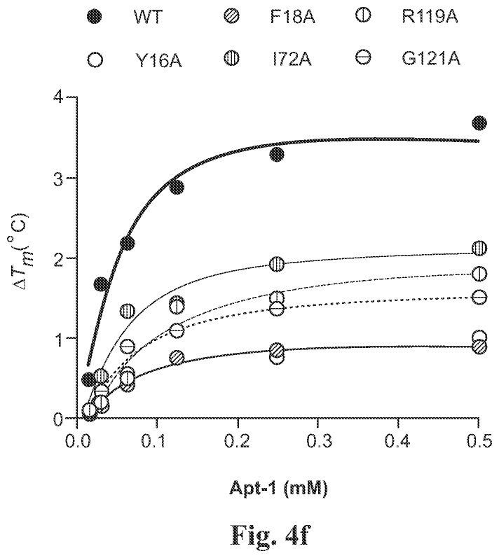

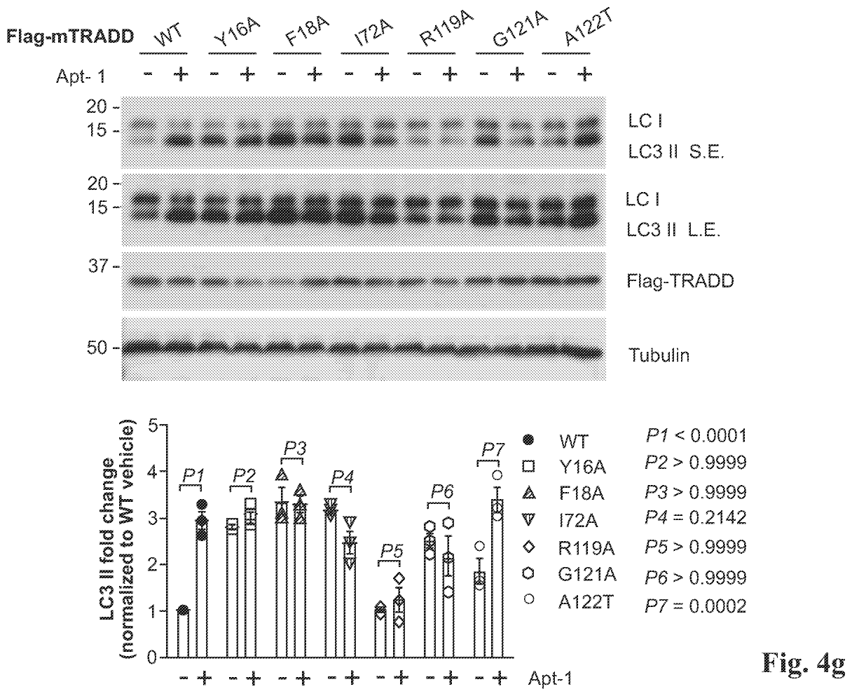

The direct binding of TRADD-N and TRAF2 was also dose-dependently reduced by Apt-1 (FIG. 4a, FIG. 15g). Binding of TRADD-N to TRAF2-C, which was competitive with TRADD-C, was dose-dependently reduced by Apt-1 as measured by a cell-free Forster resonance energy transfer (FRET)-based assay (FIG. 15h-15k).

Taken together, these data suggest a model in which TRADD-N and TRADD-C normally interact with each other; in cells stimulated by TNFα, TRADD is recruited to TNFR1 mediated by the binding of its C-terminal DD domain with the DD of TNFR1, which frees TRADD-N to interact with TRAF2 and organize the recruitment and ubiquitination of complex I. Importantly, this model suggests that ICCB-19/Apt-1 might bind to the TRADD-N interface which normally interacts with both TRADD-C and TRAF2. Consistently, Apt-1 could increase the recruitment and retention of TRADD to TNFR1 and reduce TRADD binding to TRAF2/cIAP1, thus decreasing recruitment of TRAF2/cIAP1 to complex I (FIG. 4b, FIG. 15l, 15m).

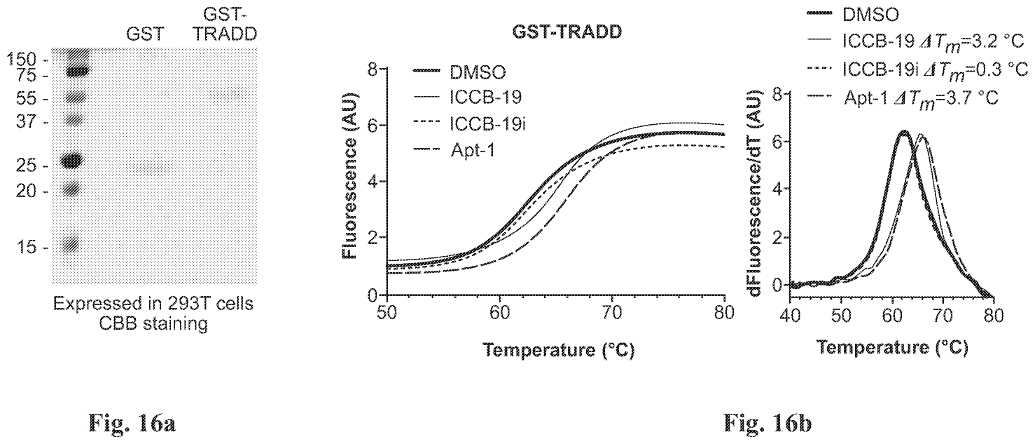

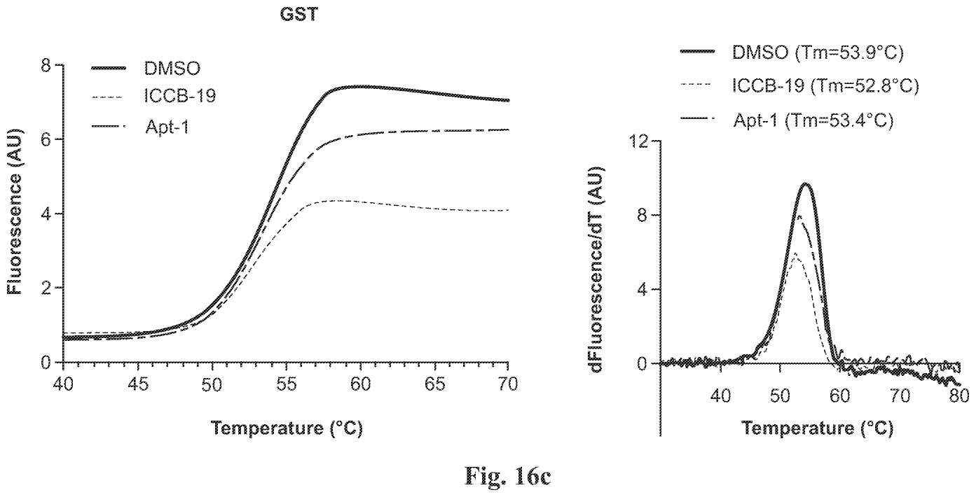

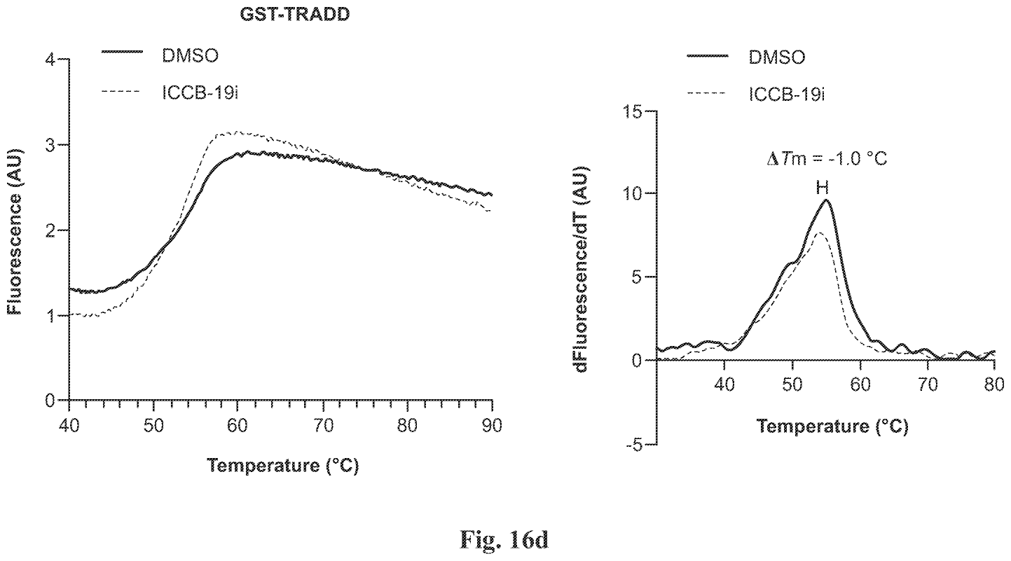

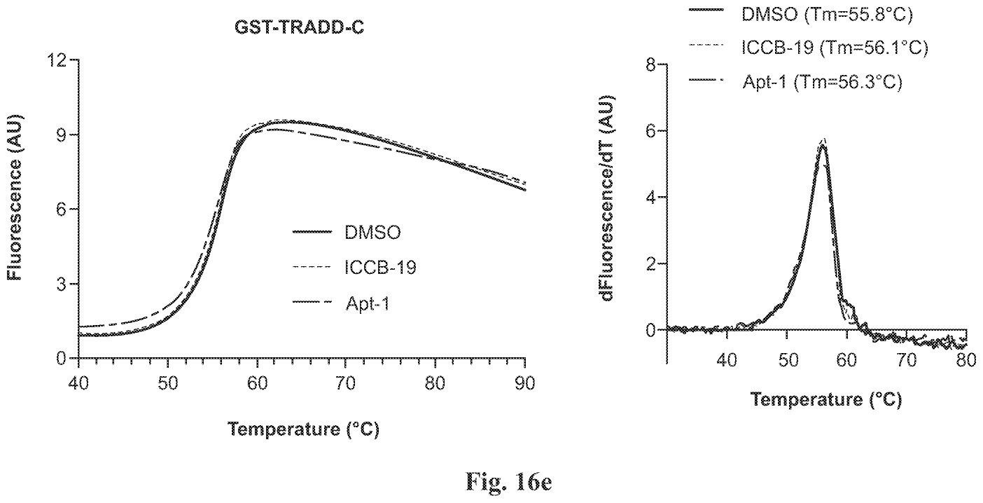

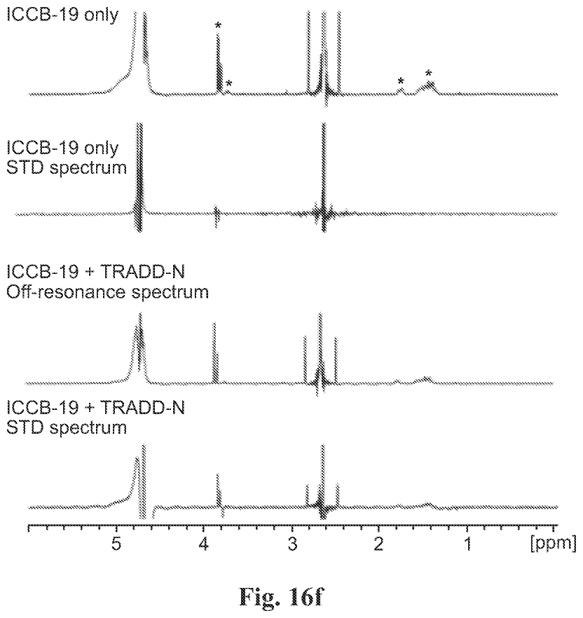

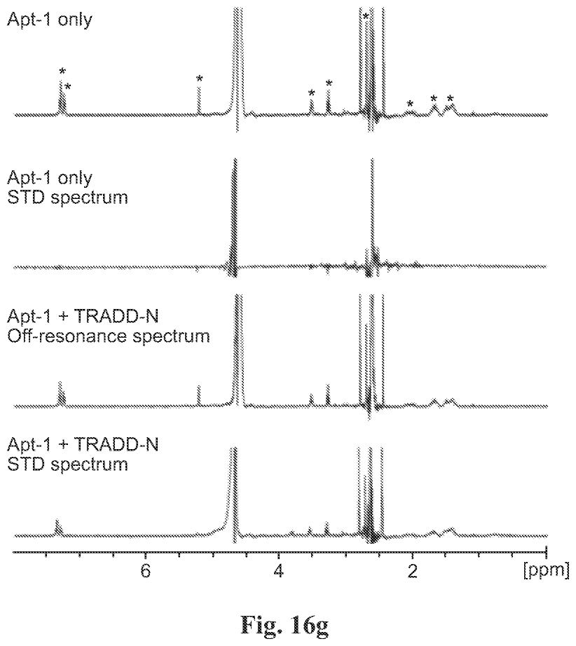

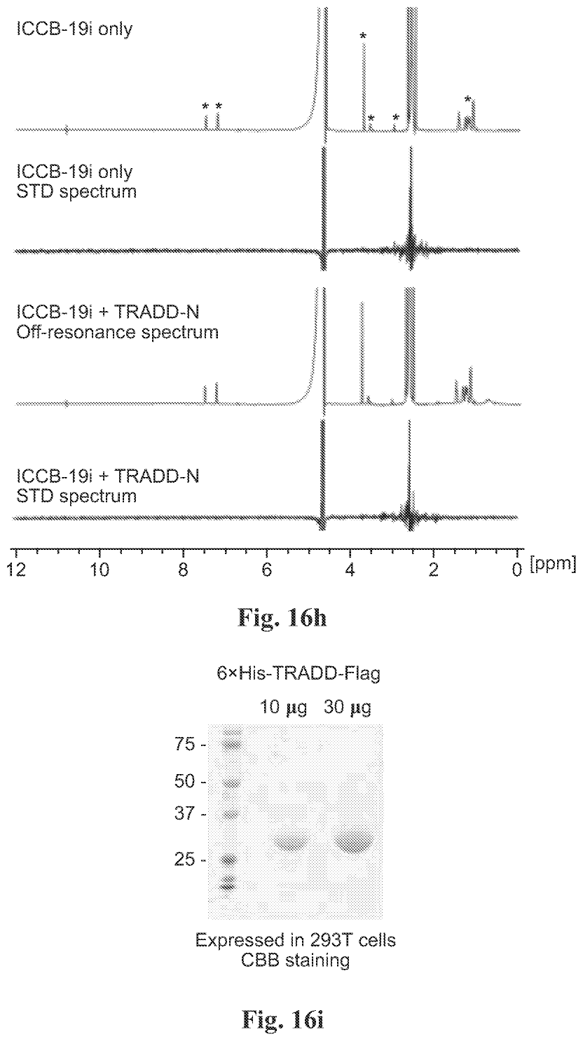

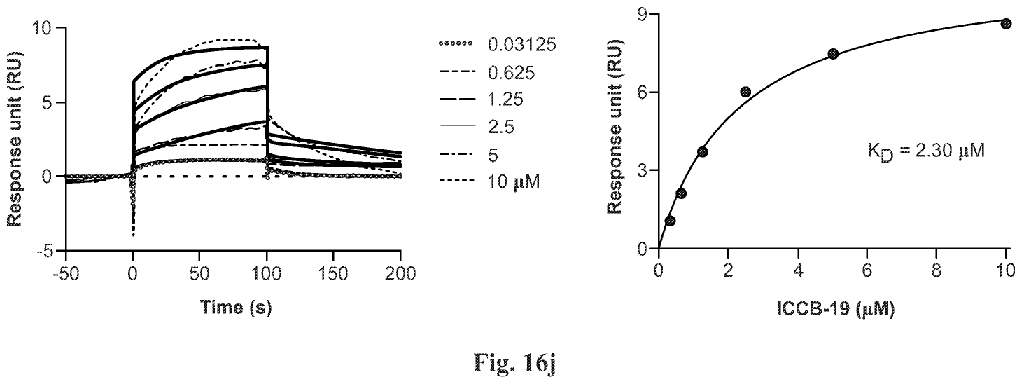

The incubation of ICCB-19/Apt-1 with GST-TRADD in a thermal shift assay increased its Tm by 3.2° C. and 3.7° C., respectively; ICCB-19i had no effect (FIG. 16a-16d). Incubation of Apt-1 with His-TRADD-N, but not GST-TRADD-C, also increased Tm by 3.7° C. (FIG. 4c, FIG. 16e), suggesting that ICCB-19/Apt-1 likely bind to TRADD-N. Saturation transfer difference (STD)-NMR confirmed that ICCB-19/Apt-1, but not ICCB-19i, could bind with TRADD-N(FIG. 16f-16i). Additionally, surface plasmon resonance (SPR) analysis determined binding K_{D} of ICCB-19/Apt-1 with TRADD-N to be 2.30 μM and 2.17 μM, respectively (FIG. 16i, j, FIG. 4d).

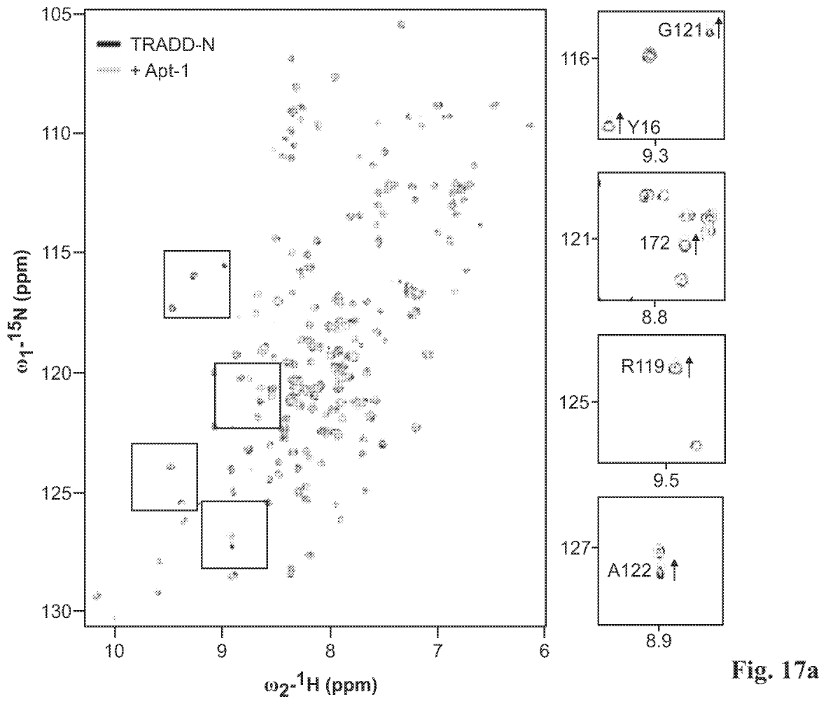

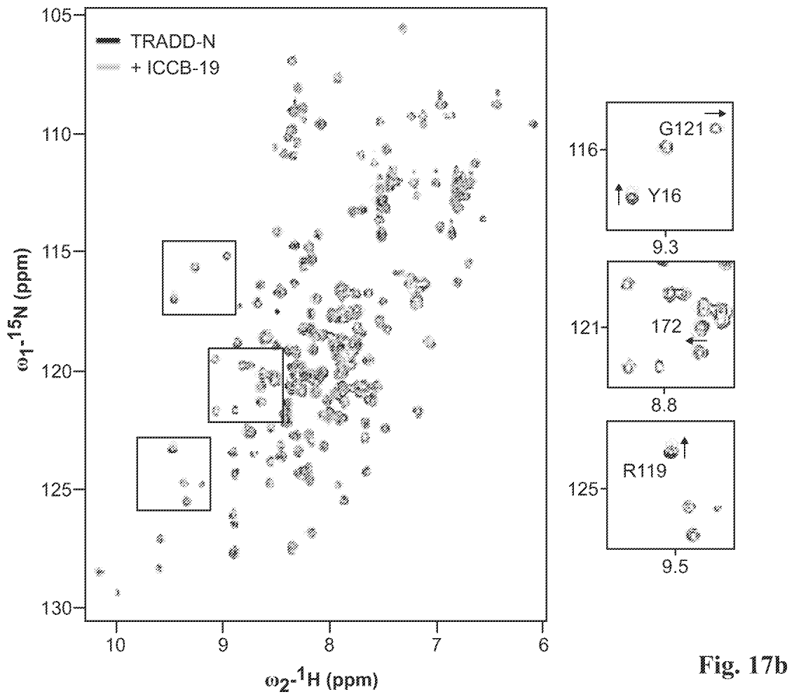

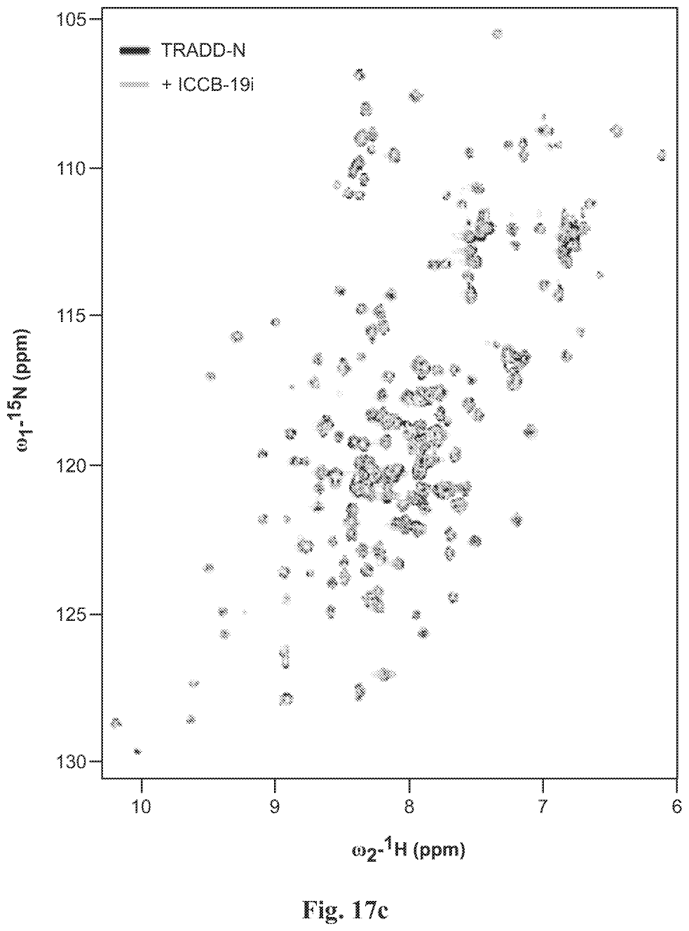

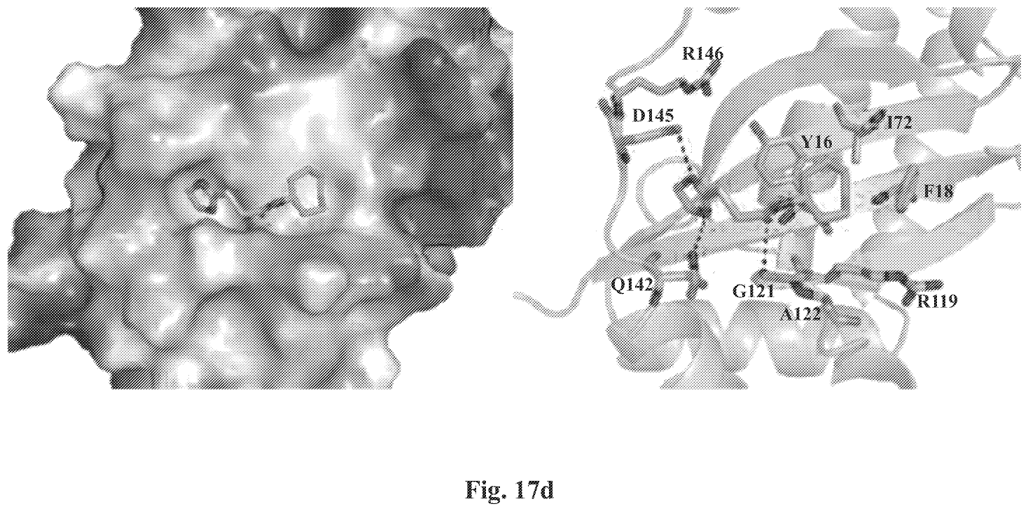

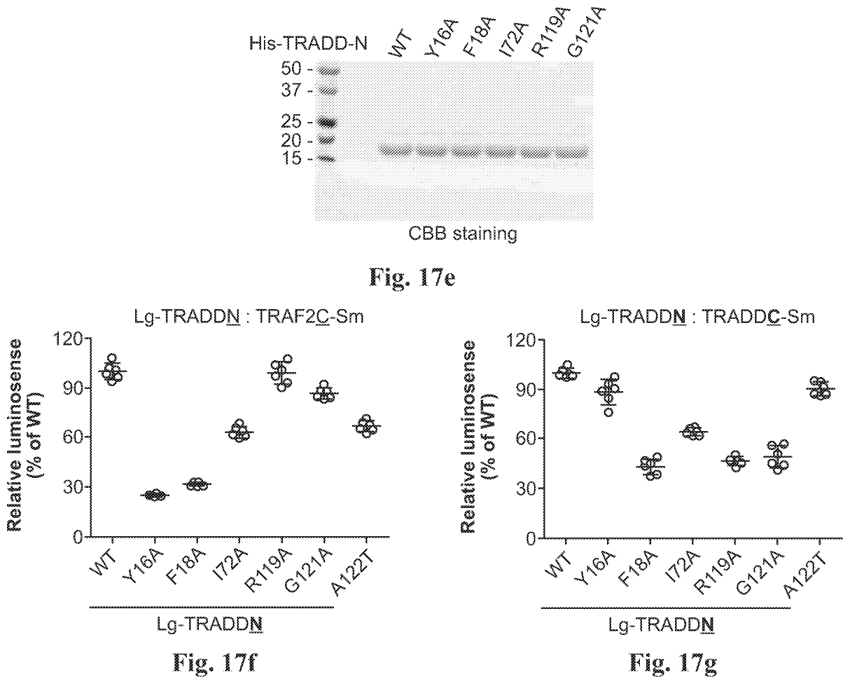

We performed a ¹H-¹⁵N heteronuclear single-quantum correlation NMR titration of Apt-1 with TRADD-N. The addition of Apt-1 into the solution of TRADD-N significantly perturbed the following residues with chemical shifts: Tyr16, Ala31, His34, Gln37, Ile72, Arg119, Gly121, Ala122, Arg124, and Arg146 (FIG. 17a). The perturbed residues localized to β-sheets 1, 3, and 4, indicating that the interface comprised of these β-sheets mediates Apt-1 binding to TRADD-N. In addition, ICCB-19, but not ICCB-19i, exhibited similar binding on TRADD-N(FIG. 17b, 17c).

Based on the NMR titration data, a structure for ICCB-19/Apt-1 bound to TRADD-N was generated using computational modeling. In this model, Apt-1/ICCB-19 bind TRADD-N with similar conformations (FIG. 4e, FIG. 17d). The ICCB-19/Apt-1 binding site occupies a part of the binding interface between TRADD-N and TRAF2-C. TRADD-N residues Tyr16, Phe18, Ile72, and Arg119 form a hydrophobic pocket which can bind the substituted cycloheptane of ICCB-19/Apt-1, consistent with our SAR study, where replacement of the seven-membered ring with rings containing 3-6 carbons significantly reduced activity (described in a separate manuscript). TRADD-N backbone amide group Gly121 forms a hydrogen bond with the carbonyl oxygen of ICCB-19/Apt-1. For the interaction between ICCB-19 and TRADD-N, TRADD-N residues Gln142 and Asp145 form two additional hydrogen bonds with the heteroatoms of 4,5-dihydro-1H-imidazole group. In addition, the phenyl group of Apt-1 forms π-π stacking with Tyrl6. Consistent with this model, binding of Apt-1 with recombinant TRADD-N mutants (Y16A, F18A, I72A, R₁ 19A, and G121A) was reduced relative to WT (FIG. 17e, FIG. 4f), demonstrating the importance of these residues in mediating TRADD-N/Apt-1 binding.

Tyr16, Phe18, and Ile72 are involved in TRADD-N-TRAF2-C binding. Consistently, Y16A, F18A, or I72A TRADD-N mutations significantly reduced its binding with TRAF2-C (FIG. 17f). In addition, Arg119, Gly121, and Ala122 sit in a region which interacts with TRAF2-C, although their functional importance is unknown. Both G121A and A122T, but not R119A, reduced TRADD-N-TRAF2-C interaction. Expression of mutant TRADD Y16A, F18A, I72A, G121A, or A122T in Tradd-deficient cells did not suppress autophagy relative to expression of WT TRADD (FIG. 4g). Apt-1 could not further enhance autophagy in Y16A, F18A, I72A, or G121A TRADD mutant-expressing cells. TRADD R119A mutant did not enhance autophagy, however Apt-1 could not induce autophagy in this mutant line (FIG. 4g), confirming that Arg119 mediates Apt-1 binding. Consistent with a predicted hydrogen bond between Gly121 and ICCB-19/Apt-1, treatment with Apt-1 could not further induce autophagy in G121A expressing Tradd-knockout MEFs (FIG. 4g). In contrast, treatment with Apt-1 in A122T expressing Tradd-knockout cells was still able to induce autophagy. These mutagenesis results suggest that TRADD-TRAF2 interaction can regulate autophagy and furthermore, ICCB-19/Apt-1 interact with some TRADD-N amino acid residues which mediate binding with TRAF2-C. Thus, disrupting the interaction of TRADD and TRAF2 may form the basis of autophagy induction mediated by Apt-1/ICCB-19.

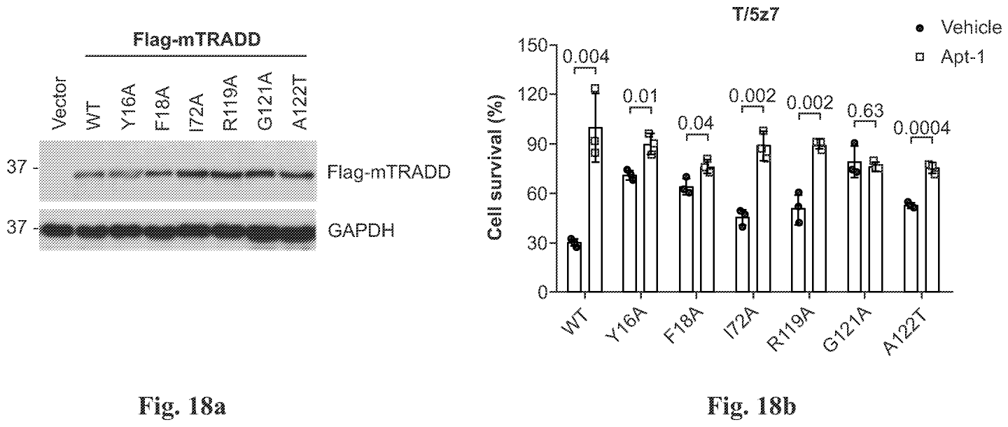

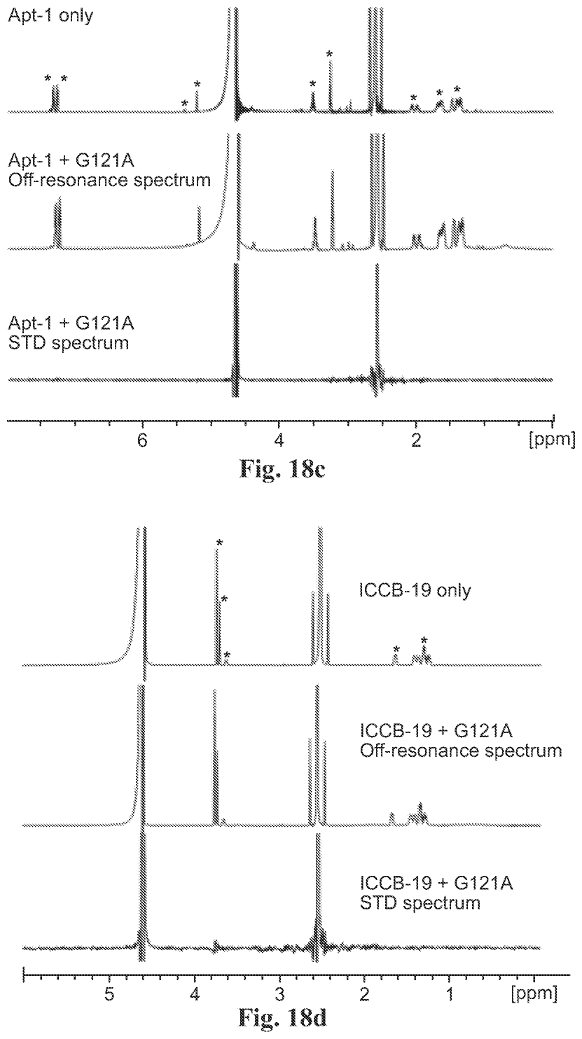

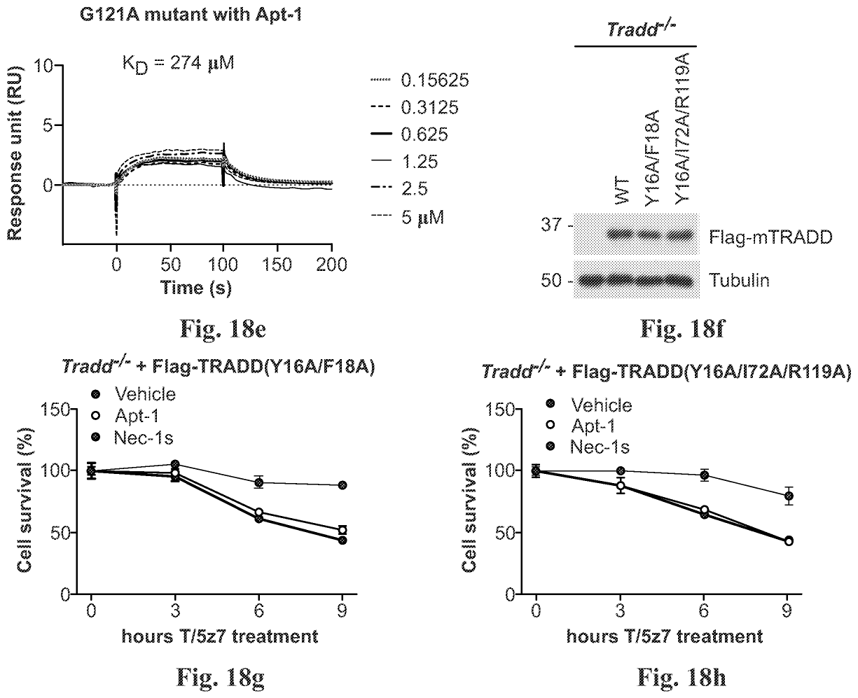

We also characterized the interaction of TRADD-N with TRADD-C in suppressing RDA. Compared to WT TRADD-N, the interactions of F18A, I72A, R119A, or G121A TRADD-N mutants with TRADD-C were all compromised to varying extents (FIG. 17g). Tradd-knockout cells complemented with Y16A, F18A, I72A, R119A, G121A, and A122T still retained partial resistance to RDA (FIG. 18a, 18b). Apt-1 was unable to provide additional protection in Tradd-knockout cells expressing G121A TRADD mutant, consistent with the inability of ICCB-19/Apt-1 to bind to G121A TRADD mutant protein in vitro (FIG. 18b-e).

Apt-1 was able to partially protect against RDA in Tradd-knockout cells complemented with Y16A, F18A, I72A, or R119A. The reconstitution of Y16A/F18A double-mutant and Y16A/I72A/R119A triple-mutant blocked RDA protection by Apt-1 (FIG. 18f-18h). Thus, the hydrophobic pocket formed by residues Tyr16, Phe18, Ile72, and Arg119 may collectively stabilize TRADD-N interaction with ICCB-19/Apt-1.

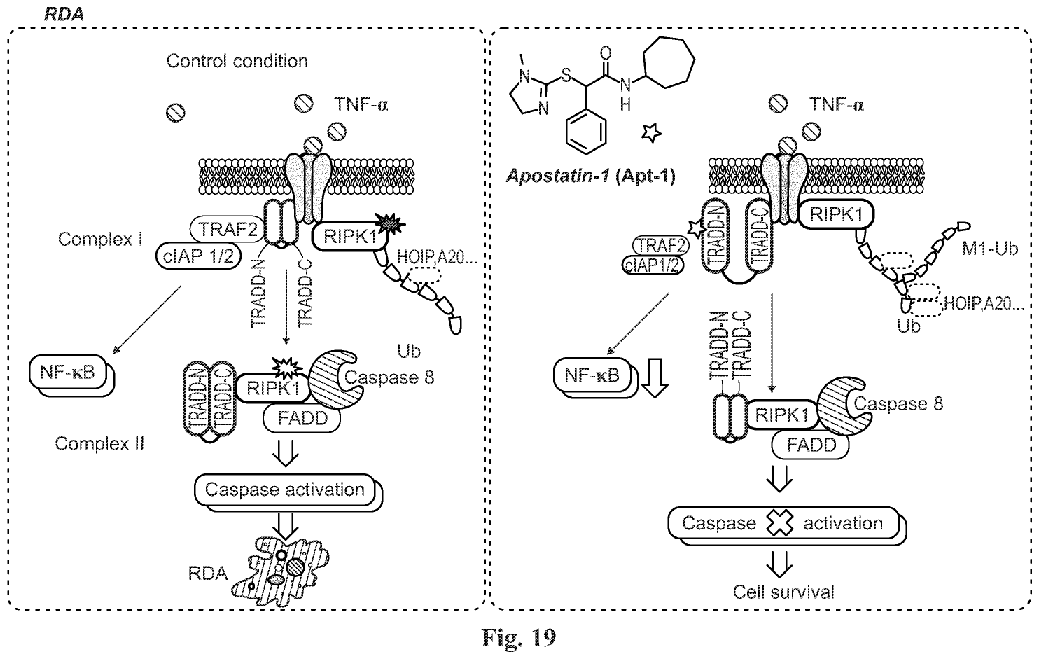

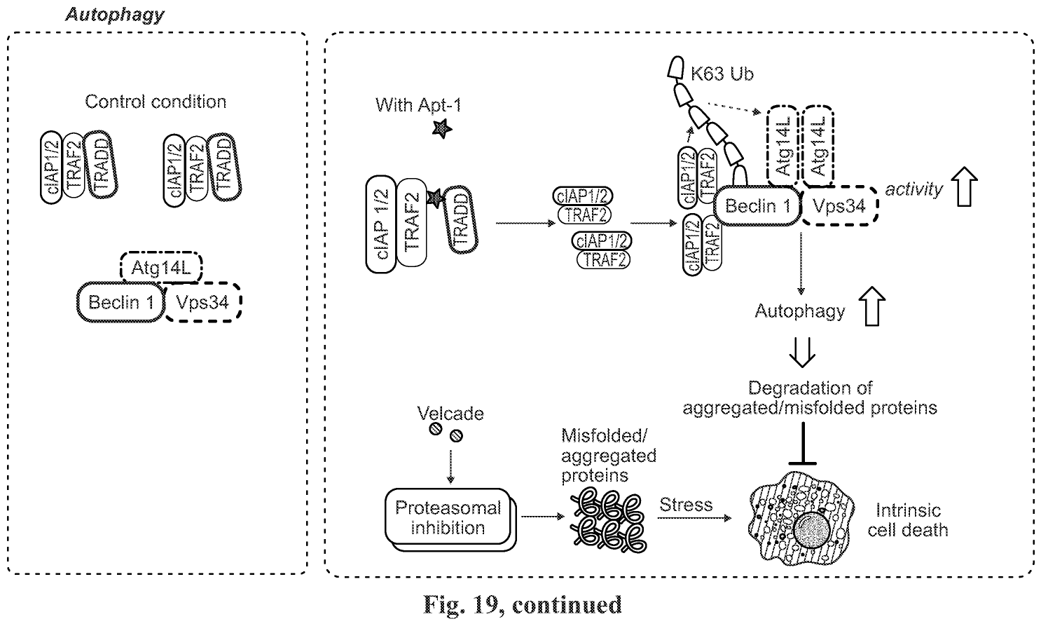

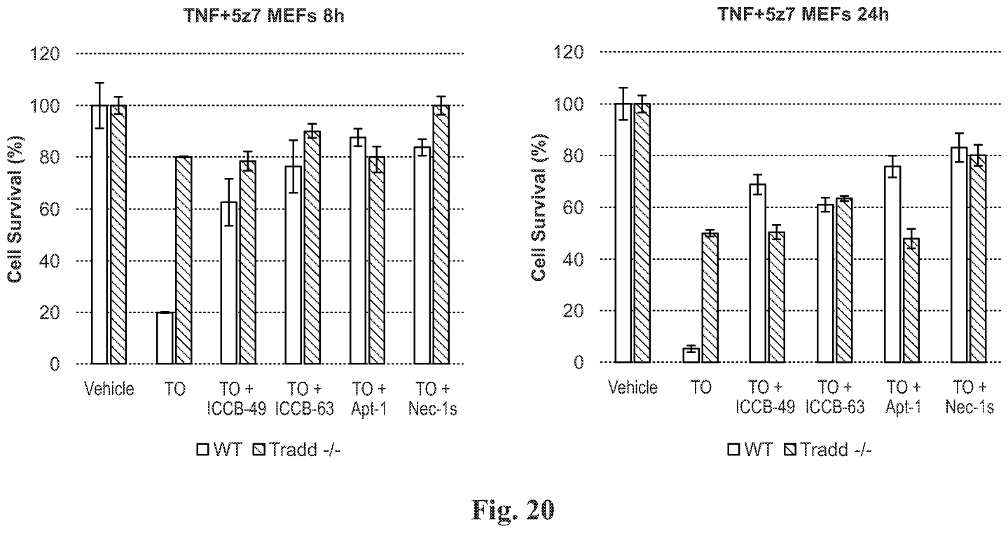

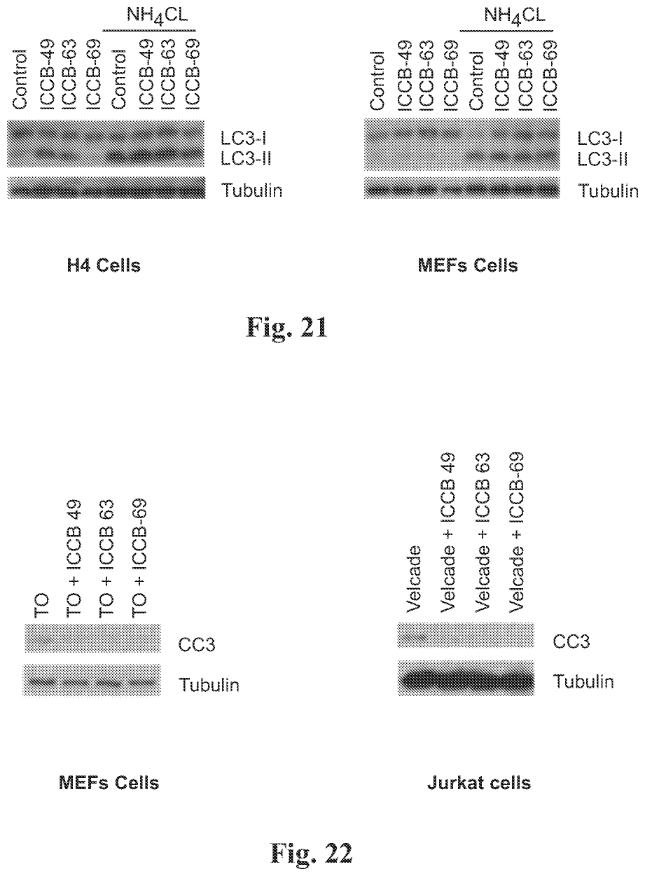

Taken together, these results suggest that ICCB-19/Apt-1 can bind with N-terminal TRAF2 binding domain of TRADD, disrupting binding to both TRAF2 and TRADD-C, to inhibit RDA and activate autophagy. Reducing the interaction of TRADD-N with TRAF2 releases TRAF2/cIAP1/2 from TRADD cytosolically, which might be primarily responsible for promoting autophagy by enhancing K63 ubiquitination of Beclin 1. In contrast, protection of RDA in TNFα stimulated cells might be attributable to both reducing interaction between TRADD-N and TRADD-C and inhibiting activation of RIPK1 in complex I by modulating its ubiquitination (FIG. 19). Disrupting TRADD-N and TRADD-C both stabilizes TRADD with TNFR1 in complex I and decreases availability of TRADD in complex IIa, where it is essential for activation of caspases.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. i) A method of treating a neurodegenerative disease, ischemic brain injury, amyloidosis, inflammatory bowel diseases, liver diseases or a metabolic disease in a subject in need thereof, comprising administering to the subject an effective amount of a compound of Formula I, or a compound selected from 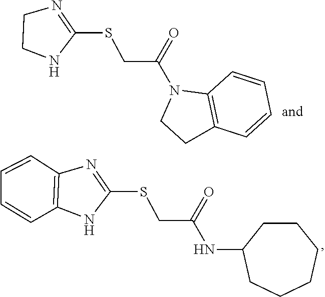 [SMILES: O=C(CSC1=NCCN1)N1CCc2ccccc21 ; O=C(CSc1nc2ccccc2[nH]1)NC1CCCCCC1]
or a pharmaceutically acceptable salt thereof; or
ii) a method of blocking apoptosis and/or inducing autophagy in a subject in need thereof, comprising administering to the subject an effective amount of a compound of Formula I, or a compound selected from 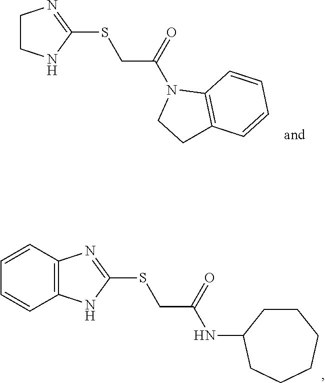 [SMILES: O=C(CSC1=NCCN1)N1CCc2ccccc21 ; O=C(CSc1nc2ccccc2[nH]1)NC1CCCCCC1]
or a pharmaceutically acceptable salt thereof; or
iii) an in vitro method of blocking apoptosis and/or inducing autophagy in a cell, comprising contacting the cell with a compound of Formula I, or a compound selected from 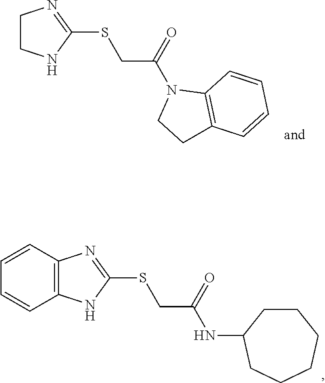 [SMILES: O=C(CSC1=NCCN1)N1CCc2ccccc21 ; O=C(CSc1nc2ccccc2[nH]1)NC1CCCCCC1]
or a pharmaceutically acceptable salt thereof; or
iv) a method of promoting cellular recruitment of TRADD to complex I in a cell, comprising contacting the cell with a compound of Formula I, or a compound selected from 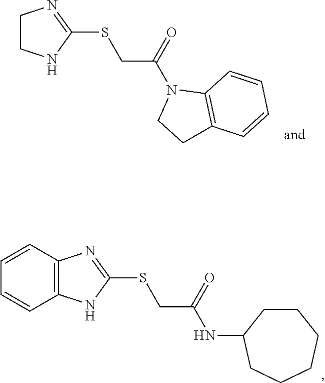 [SMILES: O=C(CSC1=NCCN1)N1CCc2ccccc21 ; O=C(CSc1nc2ccccc2[nH]1)NC1CCCCCC1]
or a pharmaceutically acceptable salt thereof:
wherein the compound of Formula I has the following structure: 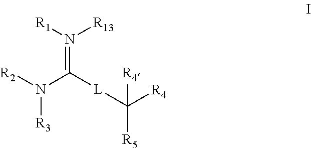
wherein
L is CH₂, NR₁ₐ, heteroaryl or S(O)n, where n is 0, 1, or 2;
R₁ₐ is independently selected from H, CN, alkyl, and aryl;
R₃ is selected from H, alkyl, and aryl;
R₄ is selected from H, alkyl, and aryl;
R_{4′} is selected from H, alkyl, and aryl;
R₅ is selected from H, alkyl, aryl, heteroaryl, —(CH₂)ₚCONR₆R₇ where p is 0, 1, or 2,
—CH₂NR₆R₇, —CH(OH)NR₆R₇, —CH(OH)CH₂-cycloalkyl, —CH(OH)CH₂-NHcycloalkyl, and —CR₁₀═CHR₁₁;
R₆ is selected from H, alkyl, C₃₋₈cycloalkyl, aryl, and -NHcycloalkyl;
R₇ is selected from H and alkyl;
or R₆ and R₇, taken together with the nitrogen atom to which they are attached, form a heterocyclyl;
R₁₀ is selected from H and halo;
R₁₁ is cycloalkyl;
R₁₃ is absent or alkyl, where the alkyl forms an iminium group; and
(a) R₁ and R₂ are each independently selected from H, CN, alkyl, and aryl; or
(b) R₁ and R₂, taken together with the atoms to which they are attached, form a heterocyclyl of Formula IA: 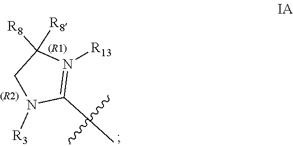
R₈ and R_{8′} are each independently selected from H, alkyl, and aryl; or
(c) R₁ and R₂, taken together with the atoms to which they are attached, and R₃ and R₄, taken together with the atoms to which they are attached, form a bicycle of Formula IB: 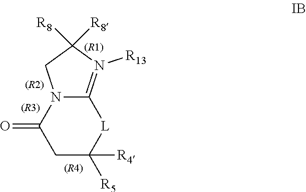
R₈ and R_{8′} are each independently selected from H, alkyl, and aryl;
further wherein when R₅ is —(CH₂)₀CONR₆R₇, then R₇ and R₄, taken together with the atoms to which they are attached, may form a heterocyclyl of Formula IC:

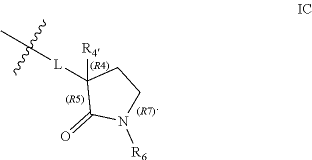

2. The method of claim 1, wherein L is S(O).

3-4. (canceled)

5. The method of claim 1, wherein L is CH₂ or oxadiazolyl.

6. The method of claim 1, wherein R₃ is selected from H, methyl, ethyl, isopropyl and phenyl.

7. The method of claim 1, wherein R₁ and R₂, taken together with the atoms to which they are attached, form a heterocyclyl of Formula IA:

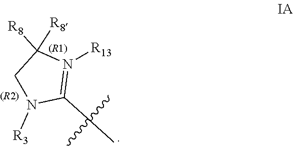

8. (canceled)

9. The method of claim 1, wherein R₁ and R₂, taken together with the atoms to which they are attached, and R₃ and R₄, taken together with the atoms to which they are attached, form a bicycle of Formula IB:

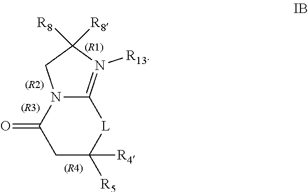

10. The method of claim 1, wherein R₅ is —(CH₂)ₚCONR₆R₇.

11-12. (canceled)

13. The method of claim 1, wherein R₅ is methyl, —C(O)NHC₃₋₈cycloalkyl, phenyl, pyrrolopyrimidinyl, or benzothiophenyl.

14. (canceled)

15. The method of claim 1, wherein R₅ is —(CH₂)₀CONR₆R₇;
R₆ is unsubstituted phenyl or phenyl substituted with one or more alkyl or alkoxy groups; and
R₇ is H.

16. The method of claim 1, wherein R₅ is —(CH₂)₀CONR₆R₇;
R₆ is cycloalkyl selected from cyclopropyl, cyclobutyl, cyclopentyl, cyclohexyl and cycloheptyl; and
R₇ is H or methyl.

17. The method of claim 1, wherein the compound of Formula I is

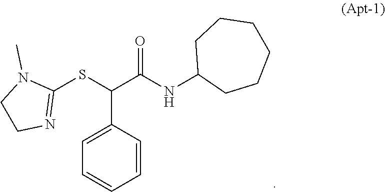 [SMILES: CN1CCN=C1SC(C(=O)NC1CCCCCC1)c1ccccc1]

18. The method of claim 1, wherein R₅ is unsubstituted phenyl or phenyl substituted with one or more of fluoro, chloro, methyl, methoxy, ethoxy, NO₂, or —CO₂Me.

19. The method of claim 1, wherein R₅ is —CR₁₀═CHR₁₁; R₁₀ is H or fluoro; and R₁₁ is cycloheptyl.

20. The method of claim 1, wherein when R₅ is —CONR₆R₇, R₇ and R₄, taken together with the atoms to which they are attached, form a heterocyclyl of Formula IC:

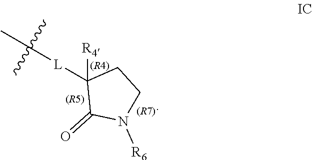

21. The method of claim 1, wherein the method is a method of treating a neurodegenerative disease; and the neurodegenerative disease is selected from Alzheimer's disease, Parkinson's disease, Huntington's disease, frontotemporal lobar degeneration, and amyotrophic lateral sclerosis.

22. The method of claim 1, wherein the method is (ii) a method of blocking apoptosis and/or inducing autophagy in a subject in need thereof or (iii) an in vitro method of blocking apoptosis and/or inducing autophagy in a cell; and the blocking apoptosis and/or inducing autophagy occurs in the presence of adapter protein TRADD.

23. A compound of Formula II: 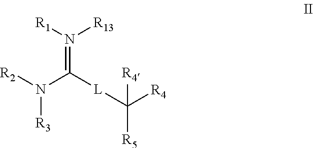
wherein
L is NR₁ₐ or S;
R₁ₐ is independently selected from CN, alkyl, and aryl;
(a) R₁ and R₂ are each independently selected from CN, alkyl, and aryl, or
(b) R₁ and R₂, taken together with the atoms to which they are attached, form a heterocyclyl of
Formula IIA: 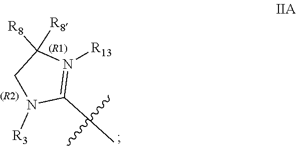
wherein R₈ and R_{8′} are each H or alkyl;
R₃ is selected from H, alkyl, and aryl;
R₄ is selected from H, alkyl, and aryl;
R₄, is selected from H, alkyl, and aryl;
R₅ is selected from aryl, —(CH₂)ₚCONR₆R₇ where p is 0 or 2, —CH₂NR₆R₇, —CH(OH)NR₆R₇, and —CR₁₀═CHR₁₁;
R₆ is selected from alkyl, aryl, and C₃₋₈cycloalkyl, such as C₇₋₈cycloalkyl or C₃₋₄cycloalkyl;
R₇ is selected from H and alkyl,
or R₆ and R₇, taken together with the nitrogen atom to which they are attached, form a heterocyclyl;
R₁₀ is H;
R₁₁ is cycloalkyl;
R₁₃ is absent or alkyl, where the alkyl forms an iminium group,
or a pharmaceutically acceptable salt thereof,
provided the compound of Formula II is not:

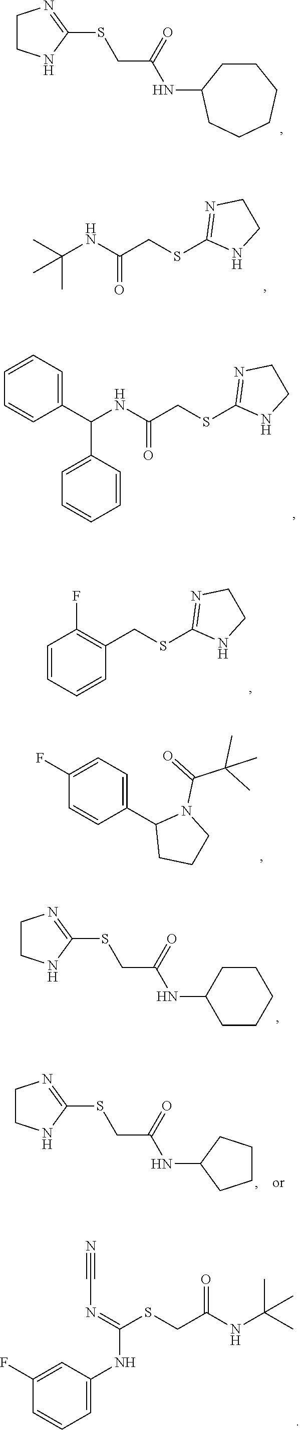

24. (canceled)

25. The compound of claim 23, wherein R₃ is selected from H, methyl, ethyl, isopropyl and phenyl.

26. The compound of claim 23, wherein R₁ and R₂, taken together with the atoms to which they are attached, form a heterocyclyl of Formula IIA:

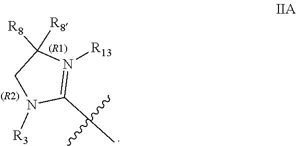

27. The compound of claim 23, wherein R₄ is selected from H, methyl, and phenyl.

28. (canceled)

29. The compound of claim 23, wherein R₅ is —(CH₂)ₚCONR₆R₇.

30. (canceled)

31. The compound of claim 23, wherein R₆ is cycloheptyl, alkyl or aryl.

32. (canceled)

33. The compound of claim 23, wherein R₇ is H or methyl.

34. The compound of claim 23, wherein R₅ is —CR₁₀═CHR₁₁; R₁₀ is H; and R₁₁ is cycloheptyl.

35. A compound selected from

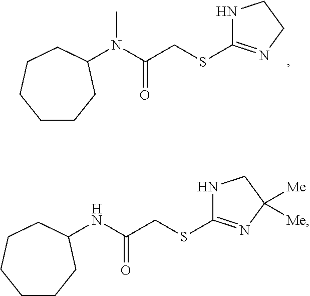 [SMILES: CN(C(=O)CSC1=NCCN1)C1CCCCCC1 ; CC1(C)CNC(SCC(=O)NC2CCCCCC2)=N1]

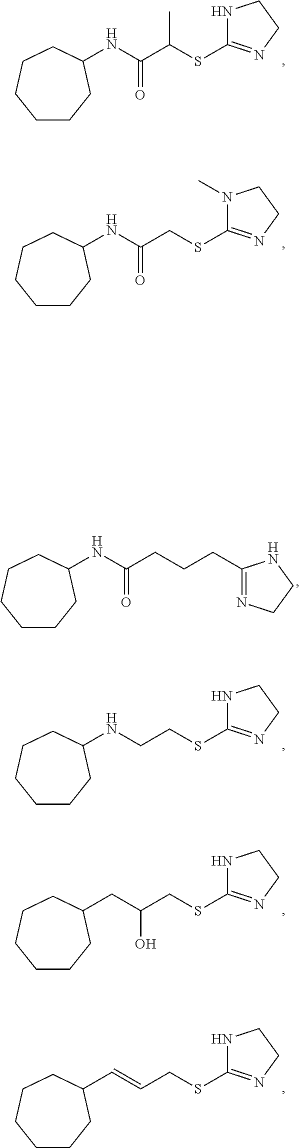 [SMILES: CC(SC1=NCCN1)C(=O)NC1CCCCCC1 ; CN1CCN=C1SCC(=O)NC1CCCCCC1 ; O=C(CCCC1=NCCN1)NC1CCCCCC1 ; C1CCCC(NCCSC2=NCCN2)CC1 ; OC(CSC1=NCCN1)CC1CCCCCC1 ; C(=C\C1CCCCCC1)/CSC1=NCCN1]

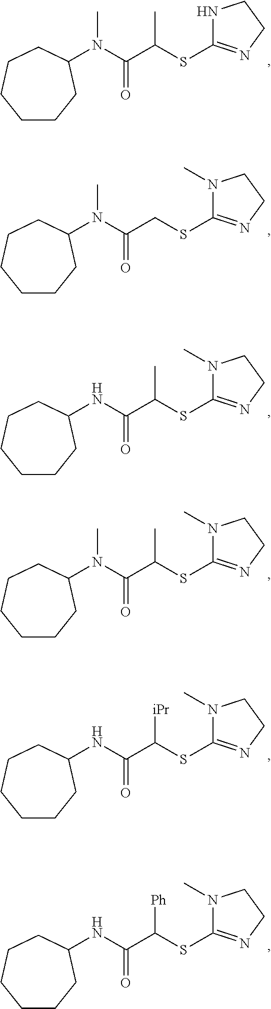 [SMILES: CC(SC1=NCCN1)C(=O)N(C)C1CCCCCC1 ; CN1CCN=C1SCC(=O)N(C)C1CCCCCC1 ; CC(SC1=NCCN1C)C(=O)NC1CCCCCC1 ; CC(SC1=NCCN1C)C(=O)N(C)C1CCCCCC1 ; CC(C)C(SC1=NCCN1C)C(=O)NC1CCCCCC1 ; CN1CCN=C1SC(C(=O)NC1CCCCCC1)c1ccccc1]

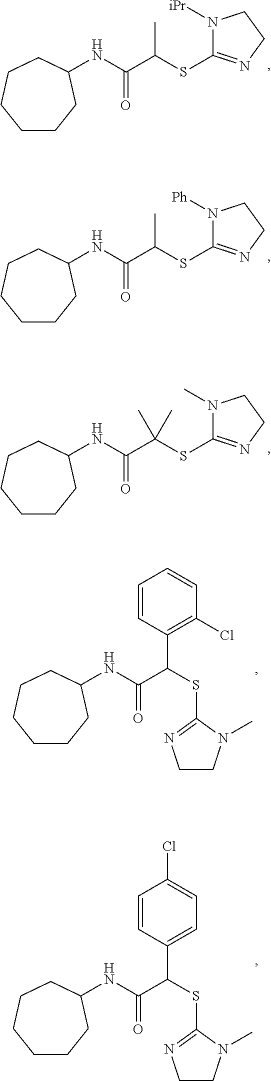 [SMILES: CC(SC1=NCCN1C(C)C)C(=O)NC1CCCCCC1 ; CC(SC1=NCCN1c1ccccc1)C(=O)NC1CCCCCC1 ; CN1CCN=C1SC(C)(C)C(=O)NC1CCCCCC1 ; CN1CCN=C1SC(C(=O)NC1CCCCCC1)c1ccccc1Cl ; CN1CCN=C1SC(C(=O)NC1CCCCCC1)c1ccc(Cl)cc1]

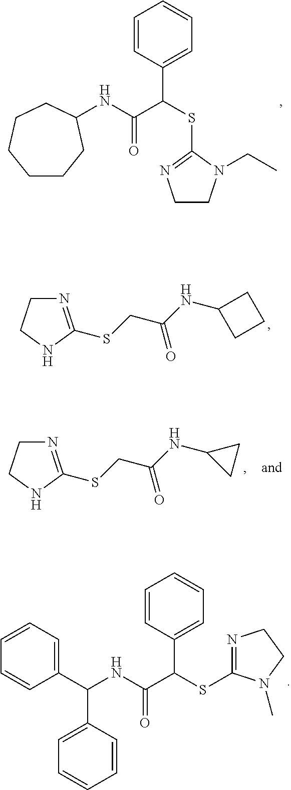 [SMILES: CCN1CCN=C1SC(C(=O)NC1CCCCCC1)c1ccccc1 ; O=C(CSC1=NCCN1)NC1CCC1 ; O=C(CSC1=NCCN1)NC1CC1 ; CN1CCN=C1SC(C(=O)NC(c1ccccc1)c1ccccc1)c1ccccc1]

36. A pharmaceutical composition comprising a compound according to claim 23, or a pharmaceutically acceptable salt thereof; and a pharmaceutically acceptable carrier.

37. A method of inhibiting TRADD in a subject in need thereof, comprising administering to the subject a therapeutically effective amount of a compound of claim 23.
